(19) United States
(12) Patent Application Publication
(10) Pub. No.: US 2019/0309307 A1
(43) Pub. Date: Oct. 10, 2019

(54) REAGENTS FOR PRODUCING T-CELLS WITH NON-FUNCTIONAL T-CELL RECEPTORS (TCRs) COMPOSITIONS COMPRISING SAME AND USE THEREOF

(71) Applicant: BENITEC BIOPHARMA LIMITED, North Sydney, New South Wales (AU)

(72) Inventors: Patty Bertha GARCIA, North Sydney (AU); Vanessa STRINGS-UFOMBAH, North Sydney (AU); Peter ROELVINK, North Sydney (AU); Michael GRAHAM, North Sydney (AU); David SUHY, North Sydney (AU)

(21) Appl. No.: 16/333,133

(22) PCT Filed: Sep. 14, 2017
(86) PCT No.: PCT/AU2017/050995

§ 371 Date: Mar. 13, 2019

Related U.S. Application Data
(60) Provisional application No. 62/394,559, filed on Sep. 14, 2016.

(51) Int. Cl. C12N 15/113; C07K 14/725; A61K 35/17; C07K 16/28; C07K 14/705

(52) U.S. Cl. CPC C12N 15/1138; C07K 14/7051; A61K 35/17; C07K 16/2803; C07K 2319/03; C07K 14/70521; C12N 2310/141; C12N 2310/531; C07K 2319/30; C07K 14/70578

(57) ABSTRACT

The present disclosure relates to reagents for producing T-cells comprising non-functional T-cell receptors (TCR), including T-cells which also express chimeric antigen receptors (CAR), i.e., CAR-T cells, compositions comprising said reagents and T-cells, and uses of said CAR-T cells in therapy e.g., adoptive therapy.

### RELATED APPLICATION DATA

The present application claims priority from U.S. Provisional Application No. 62/394,559 filed on 14 Sep. 2016, the full contents of which is incorporated herein by reference.

### TECHNICAL FIELD

The present disclosure relates to reagents for producing T-cells comprising non-functional T-cell receptors (TCR), including T-cells which also express chimeric antigen receptors (CAR), i.e., CAR-T cells, compositions comprising said reagents and T-cells, and uses of said CAR-T cells in therapy e.g., adoptive therapy.

### BACKGROUND

CAR T-Cell therapy has been an exciting advancement, particularly in the field of oncology, by providing the ability to modify a subject's own immune cells to be able to treat their cancer. Although the autologous adoptive cell transfer approach has been successfully employed in the clinic, an allogeneic approach has the potential to significantly streamline the manufacturing process. As a result, this may provide more accessible options for patients as well as enhance safety by reducing the possibility of graft-versus-host disease. Restricting expression of the TCR on the modified T-Cells helps eliminate the ability to recognize major and minor histocompatibility antigens in the recipient.

Various strategies are available for producing T-cells comprising non-functional TCRs, including CAR-T cells engineered to express CARs. However, improved strategies are needed.

### SUMMARY

The present disclosure provides a DNA-directed RNA interference (ddRNAi) construct comprising one or more nucleic acids with a DNA sequence coding for a short hairpin micro-RNA (shmiR), wherein the or each shmiR comprises:

an effector sequence of at least 17 nucleotides in length;

an effector complement sequence;

a stemloop sequence; and

a primary micro RNA (pri-miRNA) backbone;

wherein the effector sequence of the or each shmiR is substantially complementary to a region of corresponding length in a mRNA transcript for a T-cell receptor (TCR) complex subunit selected from the group consisting of: CD3-ε, TCR-α, TCR-β, CD3-γ, and CD3-δ. Exemplary mRNA transcripts for TCR complex subunit which may be targeted by shmiRs of the disclosure are described herein. Exemplary shmiR targeting mRNA transcripts for TCR complex subunits include shmiR-CD3-ε_3, shmiR-TCR-α_1, shmiR-TCR-β_5, shmiR-CD3-γ_2 and shmiR-CD3-δ_3 as described in Tables 2 and 3. Further exemplary shmiRs described in Tables 2 and 3 are also contemplated.

In one example, the ddRNAi construct comprises a nucleic acid comprising or consisting of a DNA sequence coding for shmiR-CD3-ε which comprises an effector sequence which is substantially complementary to a region of corresponding length in a mRNA transcript for the CD3-ε subunit. Exemplary shmiRs designated shmiR-CD3-ε, and nucleic acids encoding same, are described herein and shall be taken to apply mutatis mutandis to this and any other example of the disclosure describing a ddRNAi encoding a shmiR targeting CD3-ε unless specifically stated otherwise. In one particular example, the shmiR targeting CD3-ε is shmiR-CD3-ε_3.

In one example, the ddRNAi construct comprises a nucleic acid comprising or consisting of a DNA sequence coding for shmiR-TCR-α which comprises an effector sequence which is substantially complementary to a region of corresponding length in a mRNA transcript for the TCR-α subunit. Exemplary shmiRs designated shmiR-TCR-α, and nucleic acids encoding same, are described herein and shall be taken to apply mutatis mutandis to this and any other example of the disclosure describing a ddRNAi encoding a shmiR targeting TCR-α unless specifically stated otherwise. In one particular example, the shmiR targeting TCR-α is shmiR-TCR-α_1.

In one example, the ddRNAi construct comprises a nucleic acid comprising or consisting of a DNA sequence coding for shmiR-TCR-β which comprises an effector sequence which is substantially complementary to a region of corresponding length in a mRNA transcript for the TCR-β subunit. Exemplary shmiRs designated shmiR-TCR-β, and nucleic acids encoding same, are described herein and shall be taken to apply mutatis mutandis to this and any other example of the disclosure describing a ddRNAi encoding a shmiR targeting TCR-β unless specifically stated otherwise. In one particular example, the shmiR targeting TCR-β is shmiR-TCR-β_5.

In one example, the ddRNAi construct comprises a nucleic acid comprising or consisting of a DNA sequence coding for shmiR-CD3-γ which comprises an effector sequence which is substantially complementary to a region of corresponding length in a mRNA transcript for the CD3-γ subunit. Exemplary shmiRs designated shmiR-CD3-γ, and nucleic acids encoding same, are described herein and shall be taken to apply mutatis mutandis to this and any other example of the disclosure describing a ddRNAi encoding a shmiR targeting CD3-γ unless specifically stated otherwise. In one particular example, the shmiR targeting CD3-γ is shmiR-CD3-γ_2.

In one example, the ddRNAi construct comprises a nucleic acid comprising or consisting of a DNA sequence coding for shmiR-CD3-δ, which comprises an effector sequence which is substantially complementary to a region of corresponding length in a mRNA transcript for the CD3-δ subunit. Exemplary shmiRs designated shmiR-CD3-δ, and nucleic acids encoding same, are described herein and shall be taken to apply mutatis mutandis to this and any other example of the disclosure describing a ddRNAi encoding a shmiR targeting CD3-δ unless specifically stated otherwise. In one particular example, the shmiR targeting CD3-δ is shmiR-CD3-δ_3.

In one example, a DNA-directed RNA interference (ddRNAi) construct comprising two or more nucleic acids with a DNA sequence coding for a short hairpin micro-RNA (shmiR), wherein each shmiR comprises:

an effector sequence of at least 17 nucleotides in length;

an effector complement sequence;

a stemloop sequence; and

a primary micro RNA (pri-miRNA) backbone;

wherein the effector sequence of each shmiR is substantially complementary to a region of corresponding length in a mRNA transcript for a T-cell receptor (TCR) complex subunit selected from the group consisting of: CD3-ε, TCR-α, TCR-β, CD3-γ and CD3-δ.

In accordance with one example in which the ddRNAi construct comprises two or more nucleic acids with a DNA sequence coding for a shmiR, the effector sequence of each shmiR targets the mRNA transcript of a different TCR complex subunit. In accordance with another example in which the ddRNAi construct comprises two or more nucleic acids with a DNA sequence coding for a shmiR, the effector sequence of each shmiR targets the mRNA transcript of the same TCR complex subunit. In accordance with another in which the ddRNAi construct comprises at least three nucleic acids with a DNA sequence coding for a shmiR, the effector sequence of at least two shmiR targets the mRNA transcript of a different TCR complex subunit.

In each example of the ddRNAi construct described herein, each shmiR comprises, in a 5′ to 3′ direction:

a 5′ flanking sequence of the pri-miRNA backbone;

the effector complement sequence;

the stemloop sequence;

the effector sequence; and

a 3′ flanking sequence of the pri-miRNA backbone.

In one example, the stemloop sequence is the sequence set forth in SEQ ID NO: 97.

In one example, the pri-miRNA backbone is a pri-miR-30a backbone. However, other pri-miRNA backbones may be used and are described and contemplated for use herein.

In one example, the 5′ flanking sequence of the pri-miRNA backbone is set forth in SEQ ID NO: 98 and the 3′ flanking sequence of the pri-miRNA backbone is set forth in SEQ ID NO: 99.

In accordance with one example in which the ddRNAi construct comprises two or more nucleic acids with a DNA sequence coding for a shmiR, the two or more nucleic acids are selected from:

(i) a nucleic acid comprising or consisting of a DNA sequence coding for shmiR-CD3-ε which comprises an effector sequence which is substantially complementary to a region of corresponding length in a mRNA transcript for the CD3-ε subunit;
(ii) a nucleic acid comprising or consisting of a DNA sequence coding for shmiR-CD3-γ which comprises an effector sequence which is substantially complementary to a region of corresponding length in a mRNA transcript for the CD3-γ subunit; and
(iii) a nucleic acid comprising or consisting of a DNA sequence coding for shmiR-TCR-β which comprises an effector sequence which is substantially complementary to a region of corresponding length in a mRNA transcript for the TCR-β subunit.

In one example, the ddRNAi construct comprises:

(i) a nucleic acid comprising or consisting of a DNA sequence coding for shmiR-CD3-ε which comprises an effector sequence which is substantially complementary to a region of corresponding length in a mRNA transcript for the CD3-ε subunit;
(ii) a nucleic acid comprising or consisting of a DNA sequence coding for shmiR-CD3-γ which comprises an effector sequence which is substantially complementary to a region of corresponding length in a mRNA transcript for the CD3-γ subunit; and
(iii) a nucleic acid comprising or consisting of a DNA sequence coding for shmiR-TCR-β which comprises an effector sequence which is substantially complementary to a region of corresponding length in a mRNA transcript for the TCR-β subunit.

Exemplary effector sequences and cognate effector complement sequences for shmiRs targeting mRNA transcripts for TCR subunits TCR-β, CD3-γ and CD3-ε are described in Table 2 and are contemplated herein.

In one example, shmiR-CD3-ε comprises an effector sequence set forth in SEQ ID NO: 134. In one example, shmiR-TCR-β comprises an effector sequence set forth in SEQ ID NO: 116. In one example, CD3-γ comprises an effector sequence set forth in SEQ ID NO: 120.

Accordingly, in one example, the ddRNAi construct comprises at least two of:

(i) a nucleic acid comprising or consisting of a DNA sequence coding for shmiR-CD3-ε which comprises an effector sequence set forth in SEQ ID NO: 134;
(ii) a nucleic acid comprising or consisting of a DNA sequence coding for shmiR-CD3-γ which comprises an effector sequence set forth in SEQ ID NO: 120; and
(iii) a nucleic acid comprising or consisting of a DNA sequence coding for shmiR-TCR-β which comprises an effector sequence set forth in SEQ ID NO: 116.

In one example, the ddRNAi construct comprises:

(i) a nucleic acid comprising or consisting of a DNA sequence coding for shmiR-CD3-ε which comprises an effector sequence set forth in SEQ ID NO: 134;
(ii) a nucleic acid comprising or consisting of a DNA sequence coding for shmiR-CD3-γ which comprises an effector sequence set forth in SEQ ID NO: 120; and
(iii) a nucleic acid comprising or consisting of a DNA sequence coding for shmiR-TCR-β which comprises an effector sequence set forth in SEQ ID NO: 116.

In one example, shmiR-CD3-ε comprises an effector sequence set forth in SEQ ID NO: 134 and an effector complement sequence set forth in SEQ ID NO: 135. In one example, shmiR-TCR-β comprises an effector sequence set forth in SEQ ID NO: 116 and an effector complement sequence set forth in SEQ ID NO: 117. In one example, shmiR-CD3-γ comprises an effector sequence set forth in SEQ ID NO: 120 and an effector complement sequence set forth in SEQ ID NO: 121.

Accordingly, in one example, the ddRNAi construct comprises at least two of:

(i) a nucleic acid comprising or consisting of a DNA sequence coding for shmiR-CD3-ε which comprises an effector sequence set forth in SEQ ID NO: 134 and an effector complement sequence set forth in SEQ ID NO: 135;
(ii) a nucleic acid comprising or consisting of a DNA sequence coding for shmiR-CD3-γ which comprises an effector sequence set forth in SEQ ID NO: 120 and an effector complement sequence set forth in SEQ ID NO: 121; and
(iii) a nucleic acid comprising or consisting of a DNA sequence coding for shmiR-TCR-β which comprises an effector sequence set forth in SEQ ID NO: 116 and an effector complement sequence set forth in SEQ ID NO: 117.

In one example, the ddRNAi construct comprises:

(i) a nucleic acid comprising or consisting of a DNA sequence coding for shmiR-CD3-ε which comprises an effector sequence set forth in SEQ ID NO: 134 and an effector complement sequence set forth in SEQ ID NO: 135;
(ii) a nucleic acid comprising or consisting of a DNA sequence coding for shmiR-CD3-γ which comprises an effector sequence set forth in SEQ ID NO: 120 and an effector complement sequence set forth in SEQ ID NO: 121; and
(iii) a nucleic acid comprising or consisting of a DNA sequence coding for shmiR-TCR-β which comprises an effector sequence set forth in SEQ ID NO: 116 and an effector complement sequence set forth in SEQ ID NO: 117.

Exemplary shmiR sequences for shmiRs targeting mRNA transcripts for TCR subunits TCR-β, CD3-γ and CD3-ε are described in Table 3 and are contemplated herein.

In one example, shmiR-CD3-ε comprises the sequence set forth in SEQ ID NO: 153. In one example, shmiR-TCR-β comprises the sequence set forth in SEQ ID NO: 144. In one example, shmiR-CD3-γ comprises the sequence set forth in SEQ ID NO: 146.

Accordingly, in one example, the ddRNAi construct comprises at least two of:

(i) a nucleic acid comprising or consisting of a DNA sequence coding for shmiR-CD3-ε which comprises the sequence set forth in SEQ ID NO: 153;
(ii) a nucleic acid comprising or consisting of a DNA sequence coding for shmiR-CD3-γ which comprises the sequence set forth in SEQ ID NO: 146; and
(iii) a nucleic acid comprising or consisting of a DNA sequence coding for shmiR-TCR-β which comprises the sequence set forth in SEQ ID NO: 144.

In one example, the ddRNAi construct comprises:

(i) a nucleic acid comprising or consisting of a DNA sequence coding for shmiR-CD3-ε which comprises the sequence set forth in SEQ ID NO: 153;
(ii) a nucleic acid comprising or consisting of a DNA sequence coding for shmiR-CD3-γ which comprises the sequence set forth in SEQ ID NO: 146; and
(iii) a nucleic acid comprising or consisting of a DNA sequence coding for shmiR-TCR-β which comprises the sequence set forth in SEQ ID NO: 144.

In one example, the ddRNAi construct comprises or consists of a nucleic acid having DNA sequence set forth in SEQ ID NO: 175. In another example, the ddRNAi construct comprises or consists of a nucleic acid having DNA sequence set forth in SEQ ID NO: 178.

In accordance with another example in which the ddRNAi construct comprises two or more nucleic acids with a DNA sequence coding for a shmiR, the two or more nucleic acids are selected from:

(i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-TCR-β which comprises an effector sequence which is substantially complementary to a region of corresponding length in a mRNA transcript for the TCR-β subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

In one example, the ddRNAi construct comprises:

(i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-TCR-β which comprises an effector sequence which is substantially complementary to a region of corresponding length in a mRNA transcript for the TCR-β subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

Exemplary effector sequences and cognate effector complement sequences for shmiRs targeting mRNA transcripts for TCR subunits TCR-α, TCR-β and CD3-ε are described in Table 2 and are contemplated herein.

In one example, shmiR-TCR-α comprises an effector sequence set forth in SEQ ID NO: 100. In one example, shmiR-TCR-β comprises an effector sequence set forth in SEQ ID NO: 116. In one example, shmiR-CD3-ε comprises an effector sequence set forth in SEQ ID NO: 134.

Accordingly, in one example, the ddRNAi construct comprises at least two of:

(i) a nucleic acid comprising or consisting of a DNA sequence coding for shmiR-TCR-α which comprises an effector sequence set forth in SEQ ID NO: 100;
(ii) a nucleic acid comprising or consisting of a DNA sequence coding for shmiR-TCR-β which comprises an effector sequence set forth in SEQ ID NO: 116; and
(iii) a nucleic acid comprising or consisting of a DNA sequence coding for shmiR-CD3-ε which comprises an effector sequence set forth in SEQ ID NO: 134.

In one example, the ddRNAi construct comprises:

(i) a nucleic acid comprising or consisting of a DNA sequence coding for shmiR-TCR-α which comprises an effector sequence set forth in SEQ ID NO: 100;
(ii) a nucleic acid comprising or consisting of a DNA sequence coding for shmiR-TCR-β which comprises an effector sequence set forth in SEQ ID NO: 116; and
(iii) a nucleic acid comprising or consisting of a DNA sequence coding for shmiR-CD3-ε which comprises an effector sequence set forth in SEQ ID NO: 134.

In one example, shmiR-TCR-α comprises an effector sequence set forth in SEQ ID NO: 100 and an effector complement sequence set forth in SEQ ID NO: 101. In one example, shmiR-TCR-β comprises an effector sequence set forth in SEQ ID NO: 116 and an effector complement sequence set forth in SEQ ID NO: 117. In one example, shmiR-CD3-ε comprises an effector sequence set forth in SEQ ID NO: 134 and an effector complement sequence set forth in SEQ ID NO: 135.

Accordingly, in one example, the ddRNAi construct comprises at least two of:

(i) a nucleic acid comprising or consisting of a DNA sequence coding for shmiR-TCR-α which comprises an effector sequence set forth in SEQ ID NO: 100 and an effector complement sequence set forth in SEQ ID NO: 101;
(ii) a nucleic acid comprising or consisting of a DNA sequence coding for shmiR-TCR-β which comprises an effector sequence set forth in SEQ ID NO: 116 and an effector complement sequence set forth in SEQ ID NO: 117; and
(iii) a nucleic acid comprising or consisting of a DNA sequence coding for shmiR-CD3-ε which comprises an effector sequence set forth in SEQ ID NO: 134 and an effector complement sequence set forth in SEQ ID NO: 135.

In one example, the ddRNAi construct comprises:

(i) a nucleic acid comprising or consisting of a DNA sequence coding for shmiR-TCR-α which comprises an effector sequence set forth in SEQ ID NO: 100 and an effector complement sequence set forth in SEQ ID NO: 101;
(ii) a nucleic acid comprising or consisting of a DNA sequence coding for shmiR-TCR-β which comprises an effector sequence set forth in SEQ ID NO: 116 and an effector complement sequence set forth in SEQ ID NO: 117; and
(iii) a nucleic acid comprising or consisting of a DNA sequence coding for shmiR-CD3-ε which comprises an effector sequence set forth in SEQ ID NO: 134 and an effector complement sequence set forth in SEQ ID NO: 135.

Exemplary shmiR sequences for shmiRs targeting mRNA transcripts for TCR subunits TCR-α, TCR-β and CD3-ε are described in Table 3 and are contemplated herein. In one example, shmiR-TCR-α comprises the sequence set forth in SEQ ID NO: 136.

In one example, shmiR-TCR-β comprises the sequence set forth in SEQ ID NO: 144. In one example, shmiR-CD3-ε comprises the sequence set forth in SEQ ID NO: 153.

Accordingly, in one example, the ddRNAi construct comprises at least two of:

(i) a nucleic acid comprising or consisting of a DNA sequence coding for shmiR-TCR-α which comprises the sequence set forth in SEQ ID NO: 136;
(ii) a nucleic acid comprising or consisting of a DNA sequence coding for shmiR-TCR-β which comprises the sequence set forth in SEQ ID NO: 144; and
(iii) a nucleic acid comprising or consisting of a DNA sequence coding for shmiR-CD3-ε which comprises the sequence set forth in SEQ ID NO: 153.

In one example, the ddRNAi construct comprises:

(i) a nucleic acid comprising or consisting of a DNA sequence coding for shmiR-TCR-α which comprises the sequence set forth in SEQ ID NO: 136;
(ii) a nucleic acid comprising or consisting of a DNA sequence coding for shmiR-TCR-β which comprises the sequence set forth in SEQ ID NO: 144; and
(iii) a nucleic acid comprising or consisting of a DNA sequence coding for shmiR-CD3-ε which comprises the sequence set forth in SEQ ID NO: 153.

In one example, the ddRNAi construct comprises or consists of a nucleic acid having DNA sequence set forth in SEQ ID NO: 172. In another example, the ddRNAi construct comprises or consists of a nucleic acid having DNA sequence set forth in SEQ ID NO: 176.

In accordance with another example in which the ddRNAi construct comprises two or more nucleic acids with a DNA sequence coding for a shmiR, the two or more nucleic acids are selected from:

(i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-CD3-γ which comprises an effector sequence which is substantially complementary to a region of corresponding length in a mRNA transcript for the CD3-γ subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

In one example, the ddRNAi construct comprises:

(i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-CD3-γ which comprises an effector sequence which is substantially complementary to a region of corresponding length in a mRNA transcript for the CD3-γ subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

Exemplary effector sequences and cognate effector complement sequences for shmiRs targeting mRNA transcripts for TCR subunits TCR-α, CD3-γ and CD3-ε are described in Table 2 and are contemplated herein.

In one example, shmiR-TCR-α comprises an effector sequence set forth in SEQ ID NO: 100. In one example, CD3-γ comprises an effector sequence set forth in SEQ ID NO: 120. In one example, shmiR-CD3-ε comprises an effector sequence set forth in SEQ ID NO: 134.

Accordingly, in one example, the ddRNAi construct comprises at least two of:

(i) a nucleic acid comprising or consisting of a DNA sequence coding for shmiR-TCR-α which comprises an effector sequence set forth in SEQ ID NO: 100;
(ii) a nucleic acid comprising or consisting of a DNA sequence coding for shmiR-CD3-γ which comprises an effector sequence set forth in SEQ ID NO: 120; and
(iii) a nucleic acid comprising or consisting of a DNA sequence coding for shmiR-CD3-ε which comprises an effector sequence set forth in SEQ ID NO: 134.

In one example, the ddRNAi construct comprises:

(i) a nucleic acid comprising or consisting of a DNA sequence coding for shmiR-TCR-α which comprises an effector sequence set forth in SEQ ID NO: 100;
(ii) a nucleic acid comprising or consisting of a DNA sequence coding for shmiR-CD3-γ which comprises an effector sequence set forth in SEQ ID NO: 120; and
(iii) a nucleic acid comprising or consisting of a DNA sequence coding for shmiR-CD3-ε which comprises an effector sequence set forth in SEQ ID NO: 134.

In one example, shmiR-TCR-α comprises an effector sequence set forth in SEQ ID NO: 100 and an effector complement sequence set forth in SEQ ID NO: 101. In one example, shmiR-CD3-γ comprises an effector sequence set forth in SEQ ID NO: 120 and an effector complement sequence set forth in SEQ ID NO: 121. In one example, shmiR-CD3-ε comprises an effector sequence set forth in SEQ ID NO: 134 and an effector complement sequence set forth in SEQ ID NO: 135

Accordingly, in one example, the ddRNAi construct comprises at least two of:

(i) a nucleic acid comprising or consisting of a DNA sequence coding for shmiR-TCR-α which comprises an effector sequence set forth in SEQ ID NO: 100 and an effector complement sequence set forth in SEQ ID NO: 101;
(ii) a nucleic acid comprising or consisting of a DNA sequence coding for shmiR-CD3-γ which comprises an effector sequence set forth in SEQ ID NO: 120 and an effector complement sequence set forth in SEQ ID NO: 121; and
(iii) a nucleic acid comprising or consisting of a DNA sequence coding for shmiR-CD3-ε which comprises an effector sequence set forth in SEQ ID NO: 134 and an effector complement sequence set forth in SEQ ID NO: 135

In one example, the ddRNAi construct comprises:

(i) a nucleic acid comprising or consisting of a DNA sequence coding for shmiR-TCR-α which comprises an effector sequence set forth in SEQ ID NO: 100 and an effector complement sequence set forth in SEQ ID NO: 101;
(ii) a nucleic acid comprising or consisting of a DNA sequence coding for shmiR-CD3-γ which comprises an effector sequence set forth in SEQ ID NO: 120 and an effector complement sequence set forth in SEQ ID NO:121; and
(iii) a nucleic acid comprising or consisting of a DNA sequence coding for shmiR-CD3-ε which comprises an effector sequence set forth in SEQ ID NO: 134 and an effector complement sequence set forth in SEQ ID NO: 135.

Exemplary shmiR sequences for shmiRs targeting mRNA transcripts for TCR subunits TCR-α, CD3-γ and CD3-ε are described in Table 3 and are contemplated herein.

In one example, shmiR-TCR-α comprises the sequence set forth in SEQ ID NO: 136. In one example, shmiR-CD3-γ comprises the sequence set forth in SEQ ID NO: 146. In one example, shmiR-CD3-ε comprises the sequence set forth in SEQ ID NO: 153.

Accordingly, in one example, the ddRNAi construct comprises at least two of:

(i) a nucleic acid comprising or consisting of a DNA sequence coding for shmiR-TCR-α which comprises the sequence set forth in SEQ ID NO: 134;
(ii) a nucleic acid comprising or consisting of a DNA sequence coding for shmiR-CD3-γ which comprises the sequence set forth in SEQ ID NO: 146; and
(iii) a nucleic acid comprising or consisting of a DNA sequence coding for shmiR-CD3-ε which comprises the sequence set forth in SEQ ID NO: 153.

In one example, the ddRNAi construct comprises:

(i) a nucleic acid comprising or consisting of a DNA sequence coding for shmiR-TCR-α which comprises the sequence set forth in SEQ ID NO: 134;
(ii) a nucleic acid comprising or consisting of a DNA sequence coding for shmiR-CD3-γ which comprises the sequence set forth in SEQ ID NO: 146; and
(iii) a nucleic acid comprising or consisting of a DNA sequence coding for shmiR-CD3-ε which comprises the sequence set forth in SEQ ID NO: 153.

In one example, the ddRNAi construct comprises or consists of a nucleic acid having DNA sequence set forth in SEQ ID NO: 173. In another example, the ddRNAi construct comprises or consists of a nucleic acid having DNA sequence set forth in SEQ ID NO: 177.

In accordance with another example in which the ddRNAi construct comprises two or more nucleic acids with a DNA sequence coding for a shmiR, the two or more nucleic acids are selected from:

(i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-CD3-δ which comprises an effector sequence which is substantially complementary to a region of corresponding length in a mRNA transcript for the CD3-δ subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

In one example, the ddRNAi construct comprises:

(i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-CD3-δ which comprises an effector sequence which is substantially complementary to a region of corresponding length in a mRNA transcript for the CD3-δ subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

Exemplary effector sequences and cognate effector complement sequences for shmiRs targeting mRNA transcripts for TCR subunits TCR-α, CD3-δ and CD3-ε are described in Table 2 and are contemplated herein.

In one example, shmiR-TCR-α comprises an effector sequence set forth in SEQ ID NO: 100. In one example, CD3-δ comprises an effector sequence set forth in SEQ ID NO: 126. In one example, shmiR-CD3-ε comprises an effector sequence set forth in SEQ ID NO: 134.

Accordingly, in one example, the ddRNAi construct comprises at least two of:

(i) a nucleic acid comprising or consisting of a DNA sequence coding for shmiR-TCR-α which comprises an effector sequence set forth in SEQ ID NO: 100;
(ii) a nucleic acid comprising or consisting of a DNA sequence coding for shmiR-CD3-δ which comprises an effector sequence set forth in SEQ ID NO: 126; and
(iii) a nucleic acid comprising or consisting of a DNA sequence coding for shmiR-CD3-ε which comprises an effector sequence set forth in SEQ ID NO: 134.

In one example, the ddRNAi construct comprises:

(i) a nucleic acid comprising or consisting of a DNA sequence coding for shmiR-TCR-α which comprises an effector sequence set forth in SEQ ID NO: 100;
(ii) a nucleic acid comprising or consisting of a DNA sequence coding for shmiR-CD3-δ which comprises an effector sequence set forth in SEQ ID NO: 126; and
(iii) a nucleic acid comprising or consisting of a DNA sequence coding for shmiR-CD3-ε which comprises an effector sequence set forth in SEQ ID NO: 134.

In one example, shmiR-TCR-α comprises an effector sequence set forth in SEQ ID NO: 100 and an effector complement sequence set forth in SEQ ID NO: 101. In one example, shmiR-CD3-δ comprises an effector sequence set forth in SEQ ID NO: 126 and an effector complement sequence set forth in SEQ ID NO: 127. In one example, shmiR-CD3-ε comprises an effector sequence set forth in SEQ ID NO: 134 and an effector complement sequence set forth in SEQ ID NO: 135.

Accordingly, in one example, the ddRNAi construct comprises at least two of:

(i) a nucleic acid comprising or consisting of a DNA sequence coding for shmiR-TCR-α which comprises an effector sequence set forth in SEQ ID NO: 100 and an effector complement sequence set forth in SEQ ID NO: 101;
(ii) a nucleic acid comprising or consisting of a DNA sequence coding for shmiR-CD3-δ which comprises an effector sequence set forth in SEQ ID NO: 126 and an effector complement sequence set forth in SEQ ID NO: 127; and
(iii) a nucleic acid comprising or consisting of a DNA sequence coding for shmiR-CD3-ε which comprises an effector sequence set forth in SEQ ID NO: 134 and an effector complement sequence set forth in SEQ ID NO: 135.

In one example, the ddRNAi construct comprises:

a nucleic acid comprising or consisting of a DNA sequence coding for shmiR-TCR-α which comprises an effector sequence set forth in SEQ ID NO: 100 and an effector complement sequence set forth in SEQ ID NO: 101;
(ii) a nucleic acid comprising or consisting of a DNA sequence coding for shmiR-CD3-δ which comprises an effector sequence set forth in SEQ ID NO: 126 and an effector complement sequence set forth in SEQ ID NO: 127; and
(iii) a nucleic acid comprising or consisting of a DNA sequence coding for shmiR-CD3-ε which comprises an effector sequence set forth in SEQ ID NO:134 and an effector complement sequence set forth in SEQ ID NO: 135.

Exemplary shmiR sequences for shmiRs targeting mRNA transcripts for TCR subunits TCR-α, CD3-δ and CD3-ε are described in Table 3 and are contemplated herein.

In one example, shmiR-TCR-α comprises the sequence set forth in SEQ ID NO: 136. In one example, shmiR-CD3-δ comprises the sequence set forth in SEQ ID NO: 149. In one example, shmiR-CD3-ε comprises the sequence set forth in SEQ ID NO: 153.

Accordingly, in one example, the ddRNAi construct comprises at least two of:

a nucleic acid comprising or consisting of a DNA sequence coding for shmiR-TCR-α which comprises the sequence set forth in SEQ ID NO: 136;
(ii) a nucleic acid comprising or consisting of a DNA sequence coding for shmiR-CD3-δ which comprises the sequence set forth in SEQ ID NO: 149; and
(iii) a nucleic acid comprising or consisting of a DNA sequence coding for shmiR-CD3-ε which comprises the sequence set forth in SEQ ID NO: 153.

In one example, the ddRNAi construct comprises:

(i) a nucleic acid comprising or consisting of a DNA sequence coding for shmiR-TCR-α which comprises the sequence set forth in SEQ ID NO: 136;
(ii) a nucleic acid comprising or consisting of a DNA sequence coding for shmiR-CD3-δ which comprises the sequence set forth in SEQ ID NO: 149; and
(iii) a nucleic acid comprising or consisting of a DNA sequence coding for shmiR-CD3-ε which comprises the sequence set forth in SEQ ID NO: 153.

In one example, the ddRNAi construct comprises or consists of a nucleic acid having DNA sequence set forth in SEQ ID NO: 174.

In one example, the ddRNAi construct comprises a RNA pol III promoter upstream of each nucleic acid coding for a shmiR. For example, the or each RNA pol III promoter is selected from a U6 and a H1 promoter. For example, the or each RNA pol III promoter is a U6 promoter selected from a U6-9 promoter, a U6-1 promoter and U6-8 promoter. For example, one or more of the RNA pol III promoters is a U6 promoter selected from a U6-9 promoter, a U6-1 promoter and U6-8 promoter and one or more of the pol III promoters is a H1 promoter.

The present disclosure also provides a DNA construct comprising:

(a) a ddRNAi construct as described herein; and
(b) a chimeric antigen receptor (CAR) construct comprising nucleic acid with a DNA sequence coding for a CAR.

In one example, the CAR comprises an antigen binding domain.

In one example, the antigen binding domain is a binding protein. For example, the antigen binding domain is an antibody or an antigen binding domain thereof.

In one example, the antigen binding domain binds specifically to a tumor antigen. Exemplary tumor antigens are described herein and shall be taken to apply mutatis mutandis to this example of the disclosure. In one example, the CAR comprises an antigen binding domain which binds to CD19.

In another example, the antigen binding domain binds specifically to a virus antigen or viral-induced antigen found on the surface of an infected cell. In one example, the virus antigen is selected from the group consisting of Human cytomegalovirus (HCMV), Human immunodeficiency virus (HIV), Epstein-Barr virus (EBV), adenovirus (AdV), varicella zoster virus (VZV), influenza and BK virus (BKV), John Cunningham (JC) virus, respiratory syncytial virus (RSV), parainfluenzae, rhinovirus, human metapneumovirus, herpes simplex virus (HSV) 1, HSV II, human herpes virus (HHV) 6, HHV 8, Hepatitis A virus, Hepatitis B virus (HBV), Hepatitis C virus (HCV), hepatitis E virus, rotavirus, papillomavirus, parvovirus Ebola virus, zika virus, a hantavirus and vesicular stomatitis virus (VSV).

In one example, the DNA sequence coding for the CAR is operably-linked to a promoter comprised within the CAR construct and positioned upstream of the DNA sequence coding the CAR. In one example, the DNA construct comprises, in a 5′ to 3′ direction, the ddRNAi construct and the CAR construct. In another example, the DNA construct comprises, in a 5′ to 3′ direction, the CAR construct and the ddRNAi construct.

The present disclosure also provides an expression vector comprising a ddRNAi construct described herein or a DNA construct described herein.

The present disclosure also provides a plurality of expression vectors, wherein one of the expression vectors comprises a ddRNAi construct described herein and one of the expression vectors comprises a CAR construct of the DNA construct as described herein.

In one example, the expression vector(s) is/are a plasmid(s) or minicircle(s). In one example, the expression vector(s) is/are viral vectors selected from the group consisting of an adeno-associated viral (AAV) vector, a retroviral vector, an adenoviral (AdV) vector and a lentiviral (LV) vector.

In accordance with an example in which a plurality of expression vectors are provided, the expression vectors may be the same or different.

The present disclosure also provides a T-cell comprising a ddRNAi construct described herein or a DNA construct described herein or an expression vector or expression vectors as described herein.

In one example, a T-cell of the disclosure does not express a functional TCR. For example, the T-cell exhibits reduced cell-surface expression of at least two components of the TCR complex i.e., such that a functional TCR does not assemble.

In one example, a T cell further expresses a CAR. For example, a T-cell which does not express a functional (endogenous) TCR and which expresses a CAR is provided (also referred to herein as a CAR-T cell).

In one example, the CAR comprises an antigen binding domain.

In one example, the antigen binding domain is a binding protein. For example, the antigen binding domain is an antibody or an antigen binding domain thereof.

In one example, the antigen binding domain binds specifically to a tumor antigen. Exemplary tumor antigens are described herein and shall be taken to apply mutatis mutandis to this example of the disclosure.

In another example, the antigen binding domain binds specifically to a virus antigen or viral-induced antigen found on the surface of an infected cell. In one example, the virus antigen is selected from the group consisting of Human cytomegalovirus (HCMV), Human immunodeficiency virus (HIV), Epstein-Barr virus (EBV), adenovirus (AdV), varicella zoster virus (VZV), influenza and BK virus (BKV), John Cunningham (JC) virus, respiratory syncytial virus (RSV), parainfluenzae, rhinovirus, human metapneumovirus, herpes simplex virus (HSV) 1, HSV II, human herpes virus (HHV) 6, HHV 8, Hepatitis A virus, Hepatitis B virus (HBV), Hepatitis C virus (HCV), hepatitis E virus, rotavirus, papillomavirus, parvovirus Ebola virus, zika virus, a hantavirus and vesicular stomatitis virus (VSV).

The present disclosure also provides a composition comprising a ddRNAi construct described herein or a DNA construct described herein or an expression vector or expression vectors as described herein or a T-cell as described herein.

In one example, the composition further comprises one or more pharmaceutically acceptable carriers. In accordance with an example of a composition comprising a ddRNAi construct, a DNA construct, an expression vector or expression vectors as described herein, the carrier may be suitable for administration to cells e.g., ex vivo, in cell culture. In accordance with an example of a composition comprising T-cells as described herein, the carrier may be suitable for administration to a subject e.g., a human, in therapy. Suitable carriers are known in the art and described herein.

The present disclosure also provides a method of producing a T-cell which does not express a functional TCR, said method comprising introducing into a T-cell a ddRNAi construct described herein, a DNA construct described herein, an expression vector(s) described herein or a composition as described herein.

The present disclosure also provides a method of producing a T-cell which does not express a functional TCR but which expresses a chimeric antigen receptor (CAR), said method comprising introducing into a T-cell a DNA construct as described herein, an expression vector as described herein comprising said DNA construct, or a composition as described herein comprising said DNA construct.

The present disclosure also provides a method of inhibiting expression of two or more TCR complex subunits in a T-cell, said method comprising administering to the T-cell a ddRNAi construct described herein, a DNA construct described herein, an expression vector(s) described herein or a composition as described herein.

In each of the foregoing examples, the method may further comprise HLA typing the T-cell produced.

Each of the methods described herein may be performed ex vivo.

In one example, a T-cell is obtained from an individual or a cell bank prior to performance of the method.

In each of the example, the method may comprise performing one or more selection steps on the T-cells in order to select for a sub-population of T-cells. In one example, the method comprises culturing the T-cells in the presence of an immunosuppressant in order to select for T-cells which are resistant to the immunosuppressant.

The present disclosure also provides a for use of the T-cells described herein in therapy.

In one example, the present disclosure provide a method of preventing or treating cancer, graft versus host disease, infection, one or more autoimmune disorders, transplantation rejection, or radiation sickness in an individual in need thereof, comprising administering to said individual a CAR-T cell e.g., a T-cell which does not express a functional (endogenous) TCR and which expresses a CAR as described herein. In one example, the method comprises administering the CAR-T cell in a formulation.

In one example, the method comprises: obtaining a T-cell from an individual or cell bank; producing a CAR-T cell ex vivo by introducing into the T-cell a DNA construct as described herein, an expression vector as described herein comprising said DNA construct, or a composition as described herein comprising said DNA construct; and administering the CAR-T cell to the individual.

In one example, the T-cell e.g., CAR-T cell, which is administered to the individual is an allogeneic T-cell.

In one example, the T-cell e.g., CAR-T cell, which is administered to the individual is a non-autologous T-cell.

In one example, the T-cell e.g., CAR-T cell, which is administered to the individual is an autologous T-cell.

The present disclosure also provides a cell bank comprising a plurality of T-cells of different HLA types which do not express a functional TCR, wherein the cell bank comprises at least one T-cell described herein.

### BRIEF DESCRIPTION OF DRAWINGS

FIG. 1 illustrates the predicted secondary structure of a representative shmiR construct comprising a 5′ flanking region, an siRNA sense strand (effector complement sequence); a stem/loop junction sequence, an siRNA anti-sense strand (effector sequence), and a 3′ flanking region.

FIG. 2 illustrates the inhibition of the expression of TCR subunits by individual shmiR constructs. Percentage inhibition relative to the pSilencer control, as measured by qPCR, is plotted in bar format, with the corresponding shmiR and targeted subunit indicated below. This graph illustrates that all of the designed shmiRs downregulated the expression of their targeted subunit.

FIG. 3 provides a graphical representation of an exemplary triple shmiR construct. The construct comprises Lentiviral long terminal repeat sequences flanking three shmiR sequences, with each shmiR expressed under the control of a different polymerase-III promoter, as indicated in the figure.

FIG. 4 shows the FACS analysis of TCR display on the surface of Jurkat T cells transduced with the triple shmiR constructs of Example 3. As illustrated by the FACS plots, the triple shmiR constructs depleted the assembly of the TCR on the cell surface by approximately 95%.

FIG. 5 illustrates the inhibition of activation of Jurkat T cells, as measured by IL-2 secretion, transduced with the triple shmiR constructs. The graph plots percentage inhibition relative to the IL-2 secretion of untreated cells for each of the triple constructs analysed. The construct used is indicated in brackets below each bar, along with the subunits of TCR targeted by the construct.

FIG. 6 displays the inhibition of expression of IL-2 mRNA in Jurkat T cells transduced with the triple shmiR constructs. Expression levels of IL-2 in transduced Jurkat T cells was measured by qPCR and were compared to untreated cells to calculate percentage inhibition of expression. Constructs used and their TCR target subunits are indicated below the graph.

FIG. 7 shows the ability of the triple shmiR constructs to inhibit T cell activation by antigen presenting cells. The concentration of IL-2 secreted by transduced cells was measured by ELISA and plotted as percentage inhibition relative to untreated cells. Constructs used and their TCR target subunits are indicated below the graph.

FIG. 8 shows that the triple shmiR constructs do not disrupt TCR-independent T cell activation. The concentration of IL-2 secreted by transduced cells was measured by ELISA and plotted as a percentage relative to untreated cells. Constructs used and their TCR target subunits are indicated below the graph.

FIG. 9 demonstrates that the triple shmiR constructs do not significantly affect the cell cycle transitions of transduced cells. Cell populations in G2/M, S, or G0/G1 phases (as well as apoptotic cells) were counted using two colour FACS analysis according to a BrdU FITC assay. The percentage of the cells identified in each cell cycle phase are indicated in each bar, with the corresponding phases indicated to the right of the graph.

FIG. 10 provides an illustration of a clinical construct for the simultaneous knockdown of TCR expression and replacement with anti-CD19 chimeric antigen receptor (CAR). In this example, sequence coding for the anti-CD19 CAR is positioned upstream of the triple shmiR construct in a lentiviral vector. The CAR is expressed under the EF1 promoter and comprises an anti-CD19 scFv domain and a signalling domain. The triple shmiR construct is described in Example 3.

FIG. 11 provides next generation sequencing (NGS) data for TCRshmiRs expressed from triple constructs (A) pBL513, (B) pBL514 and (C) pBL516.

FIG. 12 provides data on copy number per cell of TCRshmiRs expressed from triple constructs (A) pBL513, (B) pBL514 and (C) pBL516, as determined by Quantimir Assay.

FIG. 13 provides the result of a luciferase reporter assay showing that each of the H1 promoter modified constructs (A) pBL528, (B) pBL529 and (C) pBL530 showed significantly increased inhibitory activity against a CD-3 epsilon reporter construct compared to the original triple constructs (pBL513, pBL514 and pBL516 respectively), which is consistent with increased expression of CD3-ε_1 shmiR from the H1 promoter modified constructs.

FIG. 14 illustrates the percentage inhibition of IL-2 in Jurkat T cells transducted with lentiviral triple constructs pBL513, pBL514, pBL516, pBL528, pBL529 or pBL530, as determined by ELISA.

FIG. 15 is a schematic diagram illustrating the triple hairpin pBL531 construct.

### KEY TO THE SEQUENCE LISTING

SEQ ID NO: 1: RNA effector sequence for shRNA designated TCR-α_1.
SEQ ID NO: 2: RNA effector complement sequence for shRNA designated TCR-α_1.
SEQ ID NO: 3: RNA effector sequence for shRNA designated TCR-α_2.
SEQ ID NO: 4: RNA effector complement sequence for shRNA designated TCR-α_2.
SEQ ID NO: 5: RNA effector sequence for shRNA designated TCR-α_3.
SEQ ID NO: 6: RNA effector complement sequence for shRNA designated TCR-α_3.
SEQ ID NO: 7: RNA effector sequence for shRNA designated TCR-α_4.
SEQ ID NO: 8: RNA effector complement sequence for shRNA designated TCR-α_4.
SEQ ID NO: 9: RNA effector sequence for shRNA designated TCR-α_5.
SEQ ID NO: 10: RNA effector complement sequence for shRNA designated TCR-α_5.
SEQ ID NO: 11: RNA effector sequence for shRNA designated TCR-α_6.
SEQ ID NO: 12: RNA effector complement sequence for shRNA designated TCR-α_6.
SEQ ID NO: 13: RNA effector sequence for shRNA designated TCR-β_1.
SEQ ID NO: 14: RNA effector complement sequence for shRNA designated TCR-β_1.
SEQ ID NO: 15: RNA effector sequence for shRNA designated TCR-β_2.
SEQ ID NO: 16: RNA effector complement sequence for shRNA designated TCR-β_2.
SEQ ID NO: 17: RNA effector sequence for shRNA designated TCR-β_3.
SEQ ID NO: 18: RNA effector complement sequence for shRNA designated TCR-β_3.
SEQ ID NO: 19: RNA effector sequence for shRNA designated TCR-β_4.
SEQ ID NO: 20: RNA effector complement sequence for shRNA designated TCR-β_4.
SEQ ID NO: 21: RNA effector sequence for shRNA designated TCR-β_5.
SEQ ID NO: 22: RNA effector complement sequence for shRNA designated TCR-β_5.
SEQ ID NO: 23: RNA effector sequence for shRNA designated TCR-β_6.
SEQ ID NO: 24: RNA effector complement sequence for shRNA designated TCR-β_6.
SEQ ID NO: 25: RNA effector sequence for shRNA designated TCR-β_7.
SEQ ID NO: 26: RNA effector complement sequence for shRNA designated TCR-β_7.
SEQ ID NO: 27: RNA effector sequence for shRNA designated TCR-β_8.
SEQ ID NO: 28: RNA effector complement sequence for shRNA designated TCR-β_8.
SEQ ID NO: 29: RNA effector sequence for shRNA designated TCR-β_9.
SEQ ID NO: 30: RNA effector complement sequence for shRNA designated TCR-β_9.
SEQ ID NO: 31: RNA effector sequence for shRNA designated CD3-ε_1.
SEQ ID NO: 32: RNA effector complement sequence for shRNA designated CD3-ε_1.
SEQ ID NO: 33: RNA effector sequence for shRNA designated CD3-ε_2.
SEQ ID NO: 34: RNA effector complement sequence for shRNA designated CD3-ε_2.
SEQ ID NO: 35: RNA effector sequence for shRNA designated CD3-ε_3.
SEQ ID NO: 36: RNA effector complement sequence for shRNA designated CD3-ε_3.
SEQ ID NO: 37: RNA effector sequence for shRNA designated CD3-ε_4.
SEQ ID NO: 38: RNA effector complement sequence for shRNA designated CD3-ε_4.
SEQ ID NO: 39: RNA effector sequence for shRNA designated CD3-ε_5.
SEQ ID NO: 40: RNA effector complement sequence for shRNA designated CD3-ε_5.
SEQ ID NO: 41: RNA effector sequence for shRNA designated CD3-ε_6.
SEQ ID NO: 42: RNA effector complement sequence for shRNA designated CD3-ε_6.
SEQ ID NO: 43: RNA effector sequence for shRNA designated CD3-ε_7.
SEQ ID NO: 44: RNA effector complement sequence for shRNA designated CD3-ε_7.
SEQ ID NO: 45: RNA effector sequence for shRNA designated CD3-ε_8.
SEQ ID NO: 46: RNA effector complement sequence for shRNA designated CD3-ε_8.
SEQ ID NO: 47: RNA effector sequence for shRNA designated CD3-ε_9.
SEQ ID NO: 48: RNA effector complement sequence for shRNA designated CD3-ε_9.
SEQ ID NO: 49: RNA effector sequence for shRNA designated CD3-ε_10.
SEQ ID NO: 50: RNA effector complement sequence for shRNA designated CD3-ε_10.
SEQ ID NO: 51: RNA effector sequence for shRNA designated CD3-ε_11.
SEQ ID NO: 52: RNA effector complement sequence for shRNA designated CD3-ε_11.
SEQ ID NO: 53: RNA effector sequence for shRNA designated CD3-ε_12.
SEQ ID NO: 54: RNA effector complement sequence for shRNA designated CD3-ε_12.
SEQ ID NO: 55: RNA effector sequence for shRNA designated CD3-ε_13.
SEQ ID NO: 56: RNA effector complement sequence for shRNA designated CD3-ε_13.
SEQ ID NO: 57: RNA effector sequence for shRNA designated CD3-δ_1.
SEQ ID NO: 58: RNA effector complement sequence for shRNA designated CD3-δ_1.
SEQ ID NO: 59: RNA effector sequence for shRNA designated CD3-δ_2.
SEQ ID NO: 60: RNA effector complement sequence for shRNA designated CD3-δ_2.
SEQ ID NO: 61: RNA effector sequence for shRNA designated CD3-δ_3.
SEQ ID NO: 62: RNA effector complement sequence for shRNA designated CD3-δ_3.
SEQ ID NO: 63: RNA effector sequence for shRNA designated CD3-δ_4.
SEQ ID NO: 64: RNA effector complement sequence for shRNA designated CD3-δ_4.
SEQ ID NO: 65: RNA effector sequence for shRNA designated CD3-δ_5.
SEQ ID NO: 66: RNA effector complement sequence for shRNA designated CD3-δ_5.
SEQ ID NO: 67: RNA effector sequence for shRNA designated CD3-δ_6.
SEQ ID NO: 68: RNA effector complement sequence for shRNA designated CD3-δ_6.
SEQ ID NO: 69: RNA effector sequence for shRNA designated CD3-δ_7.
SEQ ID NO: 70: RNA effector complement sequence for shRNA designated CD3-δ_7.
SEQ ID NO: 71: RNA effector sequence for shRNA designated CD3-δ_8.
SEQ ID NO: 72: RNA effector complement sequence for shRNA designated CD3-δ_8.
SEQ ID NO: 73: RNA effector sequence for shRNA designated CD3-δ_9.
SEQ ID NO: 74: RNA effector complement sequence for shRNA designated CD3-δ_9.
SEQ ID NO: 75: RNA effector sequence for shRNA designated CD3-δ_10.
SEQ ID NO: 76: RNA effector complement sequence for shRNA designated CD3-δ_10.
SEQ ID NO: 77: RNA effector sequence for shRNA designated CD3-δ_11.
SEQ ID NO: 78: RNA effector complement sequence for shRNA designated CD3-δ_11.
SEQ ID NO: 79: RNA effector sequence for shRNA designated CD3-δ_12.
SEQ ID NO: 80: RNA effector complement sequence for shRNA designated CD3-δ_12.
SEQ ID NO: 81: RNA effector sequence for shRNA designated CD3-δ_13.
SEQ ID NO: 82: RNA effector complement sequence for shRNA designated CD3-δ_13.
SEQ ID NO: 83: RNA effector sequence for shRNA designated CD3-γ_1.
SEQ ID NO: 84: RNA effector complement sequence for shRNA designated CD3-γ_1.
SEQ ID NO: 85: RNA effector sequence for shRNA designated CD3-γ_2.
SEQ ID NO: 86: RNA effector complement sequence for shRNA designated CD3-γ_2.
SEQ ID NO: 87: RNA effector sequence for shRNA designated CD3-γ_3.
SEQ ID NO: 88: RNA effector complement sequence for shRNA designated CD3-γ_3.
SEQ ID NO: 89: RNA effector sequence for shRNA designated CD3-γ_4.
SEQ ID NO: 90: RNA effector complement sequence for shRNA designated CD3-γ_4.
SEQ ID NO: 91: RNA effector sequence for shRNA designated CD3-γ_5.
SEQ ID NO: 92: RNA effector complement sequence for shRNA designated CD3-γ_5.
SEQ ID NO: 93: RNA effector sequence for shRNA designated CD3-γ_6.
SEQ ID NO: 94: RNA effector complement sequence for shRNA designated CD3-γ_6.
SEQ ID NO: 95: RNA effector sequence for shRNA designated CD3-γ_7.
SEQ ID NO: 96: RNA effector complement sequence for shRNA designated CD3-γ_7.
SEQ ID NO: 97: RNA stem loop sequence for shmiRs
SEQ ID NO: 98: 5′ flanking sequence of the pri-miRNA backbone.
SEQ ID NO: 99: 3′ flanking sequence of the pri-miRNA backbone
SEQ ID NO:100: RNA effector sequence for shmiR designated shmiR-TCR-α_1.
SEQ ID NO: 101: RNA effector complement sequence for shmiR designated shmiR-TCR-α_1.
SEQ ID NO: 102: RNA effector sequence for shmiR designated shmiR-TCR-α_2.
SEQ ID NO: 103: RNA effector complement sequence for shmiR designated shmiR-TCR-α_2.
SEQ ID NO: 104: RNA effector sequence for shmiR designated shmiR-TCR-α_3.
SEQ ID NO: 105: RNA effector complement sequence for shmiR designated shmiR-TCR-α_3.
SEQ ID NO: 106: RNA effector sequence for shmiR designated shmiR-TCR-α_4.
SEQ ID NO:107: RNA effector complement sequence for shmiR designated shmiR-TCR-α_4.
SEQ ID NO:108: RNA effector sequence for shmiR designated shmiR-TCR-β_1.
SEQ ID NO:109: RNA effector complement sequence for shmiR designated shmiR-TCR-β_1.
SEQ ID NO: 110: RNA effector sequence for shmiR designated shmiR-TCR-β_2.
SEQ ID NO: 111: RNA effector complement sequence for shmiR designated shmiR-TCR-β_2.
SEQ ID NO: 112: RNA effector sequence for shmiR designated shmiR-TCR-β_3.
SEQ ID NO: 113: RNA effector complement sequence for shmiR designated shmiR-TCR-β_3.
SEQ ID NO: 114: RNA effector sequence for shmiR designated shmiR-TCR-β_4.
SEQ ID NO: 115: RNA effector complement sequence for shmiR designated shmiR-TCR-β_4.
SEQ ID NO: 116: RNA effector sequence for shmiR designated shmiR-TCR-β_5.
SEQ ID NO: 117: RNA effector complement sequence for shmiR designated shmiR-TCR-β_5.
SEQ ID NO: 118: RNA effector sequence for shmiR designated shmiR-CD3-γ_1.
SEQ ID NO: 119: RNA effector complement sequence for shmiR designated shmiR-CD3-γ_1.
SEQ ID NO: 120: RNA effector sequence for shmiR designated shmiR-CD3-γ_2.
SEQ ID NO: 121: RNA effector complement sequence for shmiR designated shmiR-CD3-γ_2.
SEQ ID NO: 122: RNA effector sequence for shmiR designated shmiR-CD3-δ_1.
SEQ ID NO: 123: RNA effector complement sequence for shmiR designated shmiR-CD3-δ_1.
SEQ ID NO: 124: RNA effector sequence for shmiR designated shmiR-CD3-δ_2.
SEQ ID NO: 125: RNA effector complement sequence for shmiR designated shmiR-CD3-δ_2.
SEQ ID NO: 126: RNA effector sequence for shmiR designated shmiR-CD3-δ_3.
SEQ ID NO: 127: RNA effector complement sequence for shmiR designated shmiR-CD3-δ_3.
SEQ ID NO: 128: RNA effector sequence for shmiR designated shmiR-CD3-δ_4.
SEQ ID NO: 129: RNA effector complement sequence for shmiR shmiR-CD3-δ_4.
SEQ ID NO: 130: RNA effector sequence for shmiR designated shmiR-CD3-ε_1.
SEQ ID NO: 131: RNA effector complement sequence for shmiR designated shmiR-CD3-δ_1.
SEQ ID NO: 132: RNA effector sequence for shmiR designated shmiR-CD3-ε_2.
SEQ ID NO: 133: RNA effector complement sequence for shmiR designated shmiR-CD3-ε_2.
SEQ ID NO: 134: RNA effector sequence for shmiR designated shmiR-CD3-ε_3.
SEQ ID NO: 135: RNA effector complement sequence for shmiR designated shmiR-CD3-ε_3.
SEQ ID NO: 136: RNA sequence for shmiR designated shmiR-TCR-α_1.
SEQ ID NO: 137: RNA sequence for shmiR designated shmiR-TCR-α_2.
SEQ ID NO: 138: RNA sequence for shmiR designated shmiR-TCR-α_3.
SEQ ID NO: 139: RNA sequence for shmiR designated shmiR-TCR-α_4.
SEQ ID NO: 140: RNA sequence for shmiR designated shmiR-TCR-β_1.
SEQ ID NO: 141: RNA sequence for shmiR designated shmiR-TCR-β_2.
SEQ ID NO: 142: RNA sequence for shmiR designated shmiR-TCR-β_3.
SEQ ID NO: 143: RNA sequence for shmiR designated shmiR-TCR-β_4.
SEQ ID NO: 144: RNA sequence for shmiR designated shmiR-TCR-β_5.
SEQ ID NO: 145: RNA sequence for shmiR designated shmiR-CD3-γ_1.
SEQ ID NO: 146: RNA sequence for shmiR designated shmiR-CD3-γ_2.
SEQ ID NO: 147: RNA sequence for shmiR designated shmiR-CD3-δ_1.
SEQ ID NO: 148: RNA sequence for shmiR designated shmiR-CD3-δ_2.
SEQ ID NO: 149: RNA sequence for shmiR designated shmiR-CD3-δ_3.
SEQ ID NO: 150: RNA sequence for shmiR designated shmiR-CD3-δ_4.
SEQ ID NO: 151: RNA sequence for shmiR designated shmiR-CD3-ε_1.
SEQ ID NO: 152: RNA sequence for shmiR designated shmiR-CD3-ε_2.
SEQ ID NO: 153: RNA sequence for shmiR designated shmiR-CD3-ε_3.
SEQ ID NO: 154: DNA sequence coding for shmiR designated shmiR-TCR-α_1.
SEQ ID NO: 155: DNA sequence coding for shmiR designated shmiR-TCR-α_2.
SEQ ID NO: 156: DNA sequence coding for shmiR designated shmiR-TCR-α_3.
SEQ ID NO: 157: DNA sequence coding for shmiR designated shmiR-TCR-α_4.
SEQ ID NO: 158: DNA sequence coding for shmiR designated shmiR-TCR-β_1.
SEQ ID NO: 159: DNA sequence coding for shmiR designated shmiR-TCR-β_2.
SEQ ID NO: 160: DNA sequence coding for shmiR designated shmiR-TCR-β_3.
SEQ ID NO: 161: DNA sequence coding for shmiR designated shmiR-TCR-β_4.
SEQ ID NO: 162: DNA sequence coding for shmiR designated shmiR-TCR-β_5.
SEQ ID NO: 163: DNA sequence coding for shmiR designated shmiR-CD3-γ_1.
SEQ ID NO: 164: DNA sequence coding for shmiR designated shmiR-CD3-γ_2.
SEQ ID NO: 165: DNA sequence coding for shmiR designated shmiR-CD3-δ_1.
SEQ ID NO: 166: DNA sequence coding for shmiR designated shmiR-CD3-δ_2.
SEQ ID NO: 167: DNA sequence coding for shmiR designated shmiR-CD3-δ_3.
SEQ ID NO: 168: DNA sequence coding for shmiR designated shmiR-CD3-δ_4.
SEQ ID NO: 169: DNA sequence coding for shmiR designated shmiR-CD3-ε_1.
SEQ ID NO: 170: DNA sequence coding for shmiR designated shmiR-CD3-ε_2.
SEQ ID NO: 171: DNA sequence coding for shmiR designated shmiR-CD3-ε_3.
SEQ ID NO: 172: DNA sequence for triple construct pBL513 coding for shmiRs designated shmiR-TCR-α, shmiR-TCR-β and shmiR-CD3-ε.
SEQ ID NO: 173: DNA sequence for triple construct pBL514 coding for shmiRs designated shmiR-TCR-α, shmiR-CD3-γ and shmiR-CD3-ε.
SEQ ID NO: 174: DNA sequence for triple construct pBL515 coding for shmiRs designated shmiR-TCR-α, shmiR-CD3-δ and shmiR-CD3-ε.
SEQ ID NO: 175: DNA sequence for triple construct pBL516 coding for shmiRs designated shmiR-TCR-β, shmiR-CD3-γ and shmiR-CD3-ε.
SEQ ID NO: 176: DNA sequence for triple construct pBL528 coding for shmiRs designated shmiR-TCR-α, shmiR-TCR-β and shmiR-CD3-ε.
SEQ ID NO: 177: DNA sequence for triple construct pBL529 coding for shmiRs designated shmiR-TCR-α, shmiR-CD3-γ and shmiR-CD3-ε.
SEQ ID NO: 178: DNA sequence for triple construct pBL530 coding for shmiRs designated shmiR-TCR-β, shmiR-CD3-γ and shmiR-CD3-ε.
SEQ ID NO: 179: DNA sequence for triple construct pBL531 coding for shmiRs designated shmiR-TCR-β, shmiR-CD3-γ and shmiR-CD3-ε, and an anti-CD19 chimeric antigen receptor (CAR).
SEQ ID NO: 180: RNA sequence for human TCR-alpha mRNA transcript.
SEQ ID NO: 181: RNA sequence for mouse TCR-alpha mRNA transcript.
SEQ ID NO: 182: RNA sequence for predicted macaque TCR-alpha mRNA transcript.
SEQ ID NO: 183: RNA sequence for human TCR-beta mRNA transcript.
SEQ ID NO: 184: RNA sequence for mouse TCR-beta mRNA transcript.
SEQ ID NO: 185: RNA sequence for macaque TCR-beta mRNA transcript (constant region).
SEQ ID NO: 186: RNA sequence for human CD3-gamma mRNA transcript.
SEQ ID NO: 187: RNA sequence for mouse CD3-gamma mRNA transcript.
SEQ ID NO: 188: RNA sequence for macaque CD3-gamma mRNA transcript.
SEQ ID NO: 189: RNA sequence for human CD3-delta mRNA transcript.
SEQ ID NO: 190: RNA sequence for mouse CD3-delta mRNA transcript.
SEQ ID NO: 191: RNA sequence for macaque CD3-delta mRNA transcript.
SEQ ID NO: 192: RNA sequence for human CD3-epsilon mRNA transcript.
SEQ ID NO: 193: RNA sequence for mouse CD3-epsilon mRNA transcript.
SEQ ID NO: 194: RNA sequence for macaque CD3-epsilon mRNA transcript.

### DETAILED DESCRIPTION

### General

Throughout this specification, unless specifically stated otherwise or the context requires otherwise, reference to a single step, feature, composition of matter, group of steps or group of features or compositions of matter shall be taken to encompass one and a plurality (i.e., one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

### Selected Definitions

By “RNA” is meant a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly-produced RNA, as well as altered RNA that differs from naturally occurring RNA by the addition, deletion, substitution and/or alteration of one or more nucleotides. Such alterations can include addition of non-nucleotide material, such as to the end(s) of an siRNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA.

The term “RNA interference” or “RNAi” refers generally to RNA-dependent silencing of gene expression initiated by double stranded RNA (dsRNA) molecules in a cell's cytoplasm. The dsRNA molecule reduces or inhibits accumulation of transcription products of a target nucleic acid sequence, thereby silencing the gene or reducing expression of that gene.

As used herein, the term “double stranded RNA” or “dsRNA” refers to a RNA molecule having a duplex structure and comprising an effector sequence and an effector complement sequence which are of similar length to one another. The effector sequence and the effector complement sequence can be in a single RNA strand or in separate RNA strands. The “effector sequence” (often referred to as a “guide strand”) is substantially complementary to a target sequence, which in the present case, is a region of a TCR-α, TCR-β, CD3-γ, CD3-δ or CD3-ε. transcripts. The “effector sequence” can also be referred to as the “antisense sequence”. The “effector complement sequence” will be of sufficient complementary to the effector sequence such that it can anneal to the effector sequence to form a duplex. In this regard, the effector complement sequence will be substantially homologous to a region of target sequence. As will be apparent to the skilled person, the term “effector complement sequence” can also be referred to as the “complement of the effector sequence” or the sense sequence.

As used herein, the term “duplex” refers to regions in two complementary or substantially complementary nucleic acids (e.g., RNAs), or in two complementary or substantially complementary regions of a single-stranded nucleic acid (e.g., RNA), that form base pairs with one another, either by Watson-Crick base pairing or any other manner that allows for a stabilized duplex between the nucleotide sequences that are complementary or substantially complementary. It will be understood by the skilled person that within a duplex region, 100% complementarity is not required; substantial complementarity is allowable. Substantial complementarity may include 79% or greater complementarity. For example, a single mismatch in a duplex region consisting of 19 base pairs (i.e., 18 base pairs and one mismatch) results in 94.7% complementarity, rendering the duplex region substantially complementary. In another example, two mismatches in a duplex region consisting of 19 base pairs (i.e., 17 base pairs and two mismatches) results in 89.5% complementarity, rendering the duplex region substantially complementary. In yet another example, three mismatches in a duplex region consisting of 19 base pairs (i.e., 16 base pairs and three mismatches) results in 84.2% complementarity, rendering the duplex region substantially complementary, and so on.

The dsRNA may be provided as a hairpin or stem loop structure, with a duplex region comprised of an effector sequence and effector complement sequence linked by at least 2 nucleotide sequence which is termed a stem loop. When a dsRNA is provided as a hairpin or stem loop structure it can be referred to as a “hairpin RNA” or “short hairpin RNAi agent” or “shRNA”. Other dsRNA molecules provided in, or which give rise to, a hairpin or stem loop structure include primary miRNA transcipts (pri-miRNA) and precursor microRNA (pre-miRNA). Pre-miRNA shRNAs can be naturally produced from pri-miRNA by the action of the enzymes Drosha and Pasha which recognize and release regions of the primary miRNA transcript which form a stem-loop structure. Alternatively, the pri-miRNA transcript can be engineered to replace the natural stem-loop structure with an artificial/recombinant stem-loop structure. That is, an artificial/recombinant stem-loop structure may be inserted or cloned into a pri-miRNA backbone sequence which lacks its natural stem-loop structure. In the case of stemloop sequences engineered to be expressed as part of a pri-miRNA molecule, Drosha and Pasha recognize and release the artificial shRNA. dsRNA molecules produced using this approach are known as “shmiRNAs”, “shmiRs” or “microRNA framework shRNAs”.

As used herein, the term “complementary” with regard to a sequence refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of a RNA or DNA sequence may pair with a U in a RNA sequence or T in a DNA sequence. G can also pair with U in RNA molecules.

As used herein, the term “substantially complementary” is used to indicate a sufficient degree of complementarity or precise pairing such that stable and specific binding occurs between nucleic acid sequences e.g., between the effector sequence and the effector complement sequence or between the effector sequence and the target sequence. It is understood that the sequence of a nucleic acid need not be 100% complementary to that of its target or complement. The term encompasses a sequence complementary to another sequence with the exception of an overhang. In some cases, the sequence is complementary to the other sequence with the exception of 1-2 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4 mismatches. In accordance with an example in which a shmiR or shRNA of the disclosure comprises an effector sequence which is “substantially complementary” to a region a target sequence and contains 1, 2, 3 or 4 mismatch base(s) relative thereto, it is preferred that the mismatch(es) are not located within the region corresponding to the seed region of the shmiR or shRNA i.e., nucleotides 2-8 of the effector sequence.

The term “encoded” or “coding fr”, as used in the context of a shRNA or shmiR of the disclosure, shall be understood to mean a shmiR or shRNA which is capable of being transcribed from a DNA template. Accordingly, a nucleic acid that encodes, or codes for, a shmiR or shRNA of the disclosure will comprise a DNA sequence which serves as a template for transcription of the respective shmiR or shRNA.

The term “DNA-directed RNAi construct” or “ddRNAi construct” refers to a nucleic acid comprising DNA sequence which, when transcribed produces a shmiR or shRNA molecule (preferably a shmiR) which elicits RNAi. The ddRNAi construct may comprise a nucleic acid which is transcribed as a single RNA that is capable of self-annealing into a hairpin structure with a duplex region linked by a stem loop of at least 2 nucleotides i.e., shmiR or shRNA, or as a single RNA with multiple shmiR or shRNA, or as multiple RNA transcripts each capable of folding as a single shmiR or shRNA respectively. The ddRNAi construct may be provided within a larger “DNA construct” comprising one or more additional DNA sequences. For example, the ddRNAi construct may be provided in a DNA construct comprising a further DNA sequence coding for functional non-TCR receptor e.g., a chimeric antigen receptor. The ddRNAi construct and/or the DNA construct comprising same may be within an expression vector e.g., comprising one or more promoters.

As used herein, the term “operably-linked” or “operable linkage” (or similar) means that a coding nucleic acid sequence is linked to, or in association with, a regulatory sequence, e.g., a promoter, in a manner which facilitates expression of the coding sequence. Regulatory sequences include promoters, enhancers, and other expression control elements that are art-recognized and are selected to direct expression of the coding sequence.

A “vector” will be understood to mean a vehicle for introducing a nucleic acid into a Vectors include, but are not limited to, plasmids, phagemids, viruses, bacteria, and vehicles derived from viral or bacterial sources. A “plasmid” is a circular, double-stranded DNA molecule. A useful type of vector for use in accordance with the present disclosure is a viral vector, wherein heterologous DNA sequences are inserted into a viral genome that can be modified to delete one or more viral genes or parts thereof. Certain vectors are capable of autonomous replication in a host cell (e.g, vectors having an origin of replication that functions in the host cell). Other vectors can be stably integrated into the genome of a host cell, and are thereby replicated along with the host genome. As used herein, the term “expression vector” will be understood to mean a vector capable of expressing a RNA molecule of the disclosure.

As used herein, the term “chimeric Antigen Receptor” or alternatively a “CAR”, refers to a recombinant polypeptide construct comprising at least an extracellular antigen binding domain, a transmembrane domain and a cytoplasmic signaling domain (also referred to herein as “an intracellular signaling domain”) comprising a functional signalling domain derived from a stimulatory molecule and/or costimulatory molecule as defined below. In some examples, the domains in the CAR polypeptide construct are in the same polypeptide chain, e.g., comprise a chimeric fusion protein. In other embodiments, the domains in the CAR polypeptide construct are not contiguous with each other, e.g., are in different polypeptide chains.

As used herein, the term “antigen-binding domain” shall be understood to mean a protein or region thereof that recognizes and binds to an antigen. An exemplary antigen binding domain is one which binds to a tumor antigen or a viral antigen expressed on a cell surface.

The terms “tumor antigen” or “cancer-associated antigen” refer to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some examples, the tumor antigen is an antigen that is common to a specific proliferative disorder. In some examples, a cancer-associated antigen is a cell surface molecule that is overexpressed in a cancer cell in comparison to a normal cell, for instance, 1-35 fold over expression, 2-fold overexpression, 3-fold overexpression or more in comparison to a normal cell. In some examples, a cancer-associated antigen is a cell surface molecule that is inappropriately synthesized in the cancer cell, for instance, a molecule that contains deletions, additions or mutations in comparison to the molecule expressed on a normal cell. In some examples, a cancer-associated antigen will be expressed exclusively on the cell surface of a cancer cell, entirely or as a fragment (e.g., MHC/peptide), and not synthesized or expressed on the surface of a normal cell. Exemplary tumor antigens are described herein.

As used herein, the term “transmembrane domain” refers to a polypeptide that spans the plasma membrane. In one example, it links an extracellular sequence, e.g., a switch domain, an extracellular recognition element, e.g., an antigen binding domain, an inhibitory counter ligand binding domain or costimulatory ECD domain, to an intracellular sequence, e.g., a switch domain or an intracellular signaling domain. Exemplary transmembrane domains are described herein.

As used herein, the term “intracellular signaling domain” refers to an intracellular portion of a molecule. In some examples, the intracellular signal domain transduces the effector function signal and directs the cell to perform a specialized function. The term “effector function” refers to a specialized function of a cell. Effector function of a T cell, for example, may be cytolytic activity or helper activity including the secretion of cytokines. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal. In one example, the intracellular signaling domain may comprise a primary intracellular signaling domain. Exemplary primary intracellular signaling domains include those derived from the molecules responsible for primary stimulation, or antigen dependent simulation. In one example, the intracellular signaling domain comprises a costimulatory intracellular domain. Exemplary costimulatory intracellular signaling domains include those derived from molecules responsible for costimulatory signals, or antigen independent stimulation. For example, in the case of a CAR-T cell, a primary intracellular signaling domain can comprise cytoplasmic sequences of the T cell receptor, and a costimulatory intracellular signaling domain can comprise cytoplasmic sequence from co-receptor or costimulatory molecule.

As used herein, the term “costimulatory signaling domain” refers to an intracellular signaling domain of a molecule e.g., an endogenous molecule, of the CAR-T cell that, upon binding to its cognate counter ligand on a target cell, enhance e.g., increases, an immune effector response. A costimulatory intracellular signaling domain can be the intracellular portion of a costimulatory molecule. A “costimulatory molecule” refers to a molecule comprising a “costimulatory signaling domain.” A costimulatory intracellular signaling domain can be derived from the intracellular portion of a costimulatory molecule. The intracellular signaling domain can comprise the entire intracellular portion, or the entire native intracellular signaling domain, of the molecule from which it is derived, or a functional fragment thereof. Exemplary costimulatory molecule are described herein.

“T cells” belong to a group of white blood cells known as lymphocytes, and play a central role in cell-mediated immunity and, to a lesser degree the adaptive immune response. Generally, T cells are distinguished from other lymphocytes (e.g., B cells and natural killer cells) by the presence of T cell receptors (TCRs). T cells have diverse roles, which are accomplished by differentiation of distinct populations of T cells, recognizable by discrete gene expression profiles.

The terms “CAR-T cell”, “CART cell” or similar shall be understood to mean a T-cell comprising a chimeric antigen receptor (CAR).

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, ameliorating or palliating the disease state, and remission or improved prognosis.

As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, blood cancers e.g., leukemia, lung cancer and the like. Further exemplary cancers are described herein. The term “cancer” includes all types of cancerous growths or oncogenic processes, metastatic tissues or malignantly transformed cells, tissues, or organs, irrespective of histopathologic type or stage of invasiveness. The terms “tumor” and “cancer” are used interchangeably herein. Both terms encompass solid and liquid, e.g., diffuse or circulating, tumors. As used herein, the term “cancer” or “tumor” includes premalignant, as well as malignant cancers and tumors.

As used herein, the term “autologous” refers to any material e.g., a T-cell, derived from the same individual to whom it is later to be re-introduced e.g., during therapy.

As used herein, the term “non-autologous” refers to any material e.g., a T-cell, derived from a different individual relative to the individual to whom the material is to be introduced.

As used herein, the term “allogeneic” refers to any material e.g., a T-cell, derived from a different individual of the same species as the individual to whom the material is introduced. Two or more individuals are said to be allogeneic to one another when the genes at one or more loci are not identical. In some aspects, allogeneic material e.g., T-cells, from individuals of the same species may be sufficiently unlike genetically to interact antigenically.

A “therapeutically effective amount” is at least the minimum concentration or amount required to effect a measurable improvement in the disease or condition to be treated e.g., cancer or viral infection. The skilled person will be aware that such an amount will vary depending on, for example, the disease to be treated (e.g., in the case of cancer, the specific type of cancer and/or stage thereof, or in the case of viral infection, the type of virus) and/or the particular subject and/or the type or severity of a condition being treated. Accordingly, this term is not to be construed to limit the disclosure to a specific quantity, for example, weight or number of population of cells of the composition of the present disclosure. As used herein, the “subject” or “patient” can be a human or non-human animal suffering from, for example, cancer, graft versus host disease, infection e.g., viral infections, one or more autoimmune disorders, transplantation rejection, or radiation sickness. The “non-human animal” may be a primate, livestock (e.g., sheep, horses, cattle, pigs, donkeys), companion animal (e.g., pets such as dogs and cats), laboratory test animal (e.g., mice, rabbits, rats, guinea pigs, drosophila, C. elegans, zebrafish), performance animal (e.g., racehorses, camels, greyhounds) or captive wild animal. In one example, the subject or patient is a mammal. In a particularly preferred example, the subject or patient is a human.

The terms “inhibiting expression”, “reducing expression” or similar, in the context of a TCR, TCR complex or subunit thereof, refers to the absence, or an observable decrease in the level, of protein and/or mRNA transcript corresponding to a TCR complex or subunit thereof. The decrease or reduction does not have to be absolute, but may be a partial decrease sufficient for the TCR to be non-functional.

DNA-Directed RNA Interference (ddRNAi) Constructs

In one example, the present disclosure provides a DNA-directed RNA interference (ddRNAi) construct comprising two or more nucleic acids with a DNA sequence coding for a short hairpin micro-RNA (shmiR), wherein each shmiR comprises:

an effector sequence of at least 17 nucleotides in length;

an effector complement sequence;

a stemloop sequence; and

a primary micro RNA (pri-miRNA) backbone;

wherein the effector sequence of each shmiR is substantially complementary to a region of corresponding length in a mRNA transcript for a T-cell receptor (TCR) complex subunit selected from the group consisting of: CD3-ε, TCR-α, TCR-β, CD3-δ and CD3-γ. For example, the effector sequence of each shmiR will be less than 30 nucleotides in length. For example, suitable effector sequences may be in the range of 17-29 nucleotides in length. For example, the effector sequences will be 21 nucleotides in length. For example, the effector sequences will be 21 nucleotides in length and the effector complement sequences will be 20 nucleotides in length.

A shmiR targeting the TCR complex subunit CD3-ε will comprise an effector sequence which is substantially complementary to a region of corresponding length within an RNA transcript for the CD3-ε subunit. By way of example and non-limitation, an RNA transcript for the human, mouse and macaque CD3-ε subunit is described with reference to any one or more of SEQ ID NOs:192-194. ShmiRs targeting the CD3-ε subunit in accordance with this example are collectively referred to as “shmiR-CD3-ε”. For example, the effector sequence of shmiR-CD3-ε may be substantially complementary to a region of corresponding length within an mRNA sequence for CD3-ε subunit and contain 4 mismatch bases relative thereto. For example, the effector sequence of shmiR-CD3-ε may be substantially complementary to a region of corresponding length within an mRNA sequence for CD3-ε subunit and contain 3 mismatch bases relative thereto. For example, the effector sequence of shmiR-CD3-ε may be substantially complementary to a region of corresponding length within an mRNA sequence for CD3-ε subunit and contain 2 mismatch bases relative thereto. For example, the effector sequence of shmiR-CD3-ε may be substantially complementary to a region of corresponding length within an mRNA sequence for CD3-ε subunit and contain 1 mismatch base relative thereto. For example, the effector sequence of shmiR-CD3-ε may be 100% complementary to a region of corresponding length within an mRNA sequence for CD3-ε subunit.

A shmiR targeting the TCR complex subunit TCR-α will comprise an effector sequence which is substantially complementary to a region of corresponding length within an RNA transcript for the constant region of the TCR-α subunit. By way of example and non-limitation, RNA transcripts for the human, mouse and macaque TCR-α subunits are described with reference to any one or more of SEQ ID NOs:180-182. ShmiRs targeting the TCR-α subunit in accordance with this example are collectively referred to as “shmiR-TCR-α”. For example, the effector sequence of shmiR-TCR-α may be substantially complementary to a region of corresponding length within an mRNA sequence for the constant region of the TCR-α subunit and contain 4 mismatch bases relative thereto. For example, the effector sequence of shmiR-TCR-α may be substantially complementary to a region of corresponding length within an mRNA sequence for the constant region of the TCR-α subunit and contain 3 mismatch bases relative thereto. For example, the effector sequence of shmiR-TCR-α may be substantially complementary to a region of corresponding length within an mRNA sequence for the constant region of the TCR-α subunit and contain 2 mismatch bases relative thereto. For example, the effector sequence of shmiR-TCR-α may be substantially complementary to a region of corresponding length within an mRNA sequence for the constant region of the TCR-α subunit and contain 1 mismatch base relative thereto. For example, the effector sequence of shmiR-TCR-α may be 100% complementary to a region of corresponding length within an mRNA sequence for the constant region of the TCR-α subunit.

A shmiR targeting the TCR complex subunit TCR-β will comprise an effector sequence which is substantially complementary to a region of corresponding length within an RNA transcript for the constant region of the TCR-β subunit. By way of example and non-limitation, an RNA transcript for the human, mouse and macaque TCR-β subunit is described with reference to any one or more of SEQ ID NOs:183-185. ShmiRs targeting the TCR-β subunit in accordance with this example are collectively referred to as “shmiR-TCR-13”. For example, the effector sequence of shmiR-TCR-β may be substantially complementary to a region of corresponding length within an mRNA sequence for the constant region of the TCR-β subunit and contain 4 mismatch bases relative thereto. For example, the effector sequence of shmiR-TCR-β may be substantially complementary to a region of corresponding length within an mRNA sequence for the constant region of the TCR-β subunit and contain 3 mismatch bases relative thereto. For example, the effector sequence of shmiR-TCR-β may be substantially complementary to a region of corresponding length within an mRNA sequence for the constant region of the TCR-β subunit and contain 2 mismatch bases relative thereto. For example, the effector sequence of shmiR-TCR-β may be substantially complementary to a region of corresponding length within an mRNA sequence for the constant region of the TCR-β subunit and contain 1 mismatch base relative thereto. For example, the effector sequence of shmiR-TCR-β may be 100% complementary to a region of corresponding length within an mRNA sequence for the constant region of the TCR-β subunit.

A shmiR targeting the TCR complex subunit CD3-γ will comprise an effector sequence which is substantially complementary to a region of corresponding length within an RNA transcript for the CD3-γ subunit. By way of example and non-limitation, an RNA transcript for the human, mouse and macaque CD3-γ subunit is described with reference to any one or more of SEQ ID NOs:186-188. ShmiRs targeting the CD3-γ subunit in accordance with this example are collectively referred to as “shmiR-CD3-γ”. For example, the effector sequence of shmiR-CD3-γ may be substantially complementary to a region of corresponding length within an mRNA sequence for CD3-γ subunit and contain 4 mismatch bases relative thereto. For example, the effector sequence of shmiR-CD3-γ may be substantially complementary to a region of corresponding length within an mRNA sequence for CD3-γ subunit and contain 3 mismatch bases relative thereto. For example, the effector sequence of shmiR-CD3-γ may be substantially complementary to a region of corresponding length within an mRNA sequence for CD3-γ subunit and contain 2 mismatch bases relative thereto. For example, the effector sequence of shmiR-CD3-γ may be substantially complementary to a region of corresponding length within an mRNA sequence for CD3-γ subunit and contain 1 mismatch base relative thereto. For example, the effector sequence of shmiR-CD3-γ may be 100% complementary to a region of corresponding length within an mRNA sequence for CD3-γ subunit.

A shmiR targeting the TCR complex subunit CD3-δ will comprise an effector sequence which is substantially complementary to a region of corresponding length within an RNA transcript for the CD3-δ subunit. By way of example and non-limitation, an RNA transcript for the human, mouse and macaque CD3-δ subunit is described with reference to any one or more of SEQ ID NOs:189-191. ShmiRs targeting the CD3-δ subunit in accordance with this example are collectively referred to as “shmiR-CD3-δ”. For example, the effector sequence of shmiR-CD3-δ may be substantially complementary to a region of corresponding length within an mRNA sequence for CD3-δ subunit and contain 4 mismatch bases relative thereto. For example, the effector sequence of shmiR-CD3-δ may be substantially complementary to a region of corresponding length within an mRNA sequence for CD3-δ subunit and contain 3 mismatch bases relative thereto. For example, the effector sequence of shmiR-CD3-δ may be substantially complementary to a region of corresponding length within an mRNA sequence for CD3-δ subunit and contain 2 mismatch bases relative thereto. For example, the effector sequence of shmiR-CD3-δ may be substantially complementary to a region of corresponding length within an mRNA sequence for CD3-δ subunit and contain 1 mismatch base relative thereto. For example, the effector sequence of shmiR-CD3-δ may be 100% complementary to a region of corresponding length within an mRNA sequence for CD3-δ subunit.

In one example, shmiR-CD3-ε as described herein comprises: (i) an effector sequence which is substantially complementary to the sequence set forth in SEQ ID NO:135 with the exception of 1, 2, 3 or 4 base mismatches, provided that the effector sequence is capable of forming a duplex with a sequence set forth in SEQ ID NO:135; and (ii) an effector complement sequence comprising a sequence which is substantially complementary to the effector sequence. For example, shmiR-CD3-ε may comprise an effector sequence set forth in SEQ ID NO:134 and an effector complement sequence which is substantially complementary to the sequence set forth in SEQ ID NO:134 and capable of forming a duplex therewith. The effector complement sequence which is substantially complementary to the sequence set forth in SEQ ID NO:134 may be the sequence set forth in SEQ ID NO:135. A shmiR targeting the CD3-ε subunit in accordance with this example is hereinafter designated “shmiR-CD3-ε_3”.

In one example, shmiR-CD3-ε as described herein comprises: (i) an effector sequence which is substantially complementary to the sequence set forth in SEQ ID NO:131 with the exception of 1, 2, 3 or 4 base mismatches, provided that the effector sequence is capable of forming a duplex with a sequence set forth in SEQ ID NO:131; and (ii) an effector complement sequence comprising a sequence which is substantially complementary to the effector sequence. For example, shmiR-CD3-ε may comprise an effector sequence set forth in SEQ ID NO:130 and an effector complement sequence which is substantially complementary to the sequence set forth in SEQ ID NO:130 and capable of forming a duplex therewith. The effector complement sequence which is substantially complementary to the sequence set forth in SEQ ID NO:130 may be the sequence set forth in SEQ ID NO:131. A shmiR targeting the CD3-ε subunit in accordance with this example is hereinafter designated “shmiR-CD3-ε_1”.

In one example, shmiR-CD3-ε as described herein comprises: (i) an effector sequence which is substantially complementary to the sequence set forth in SEQ ID NO:133 with the exception of 1, 2, 3 or 4 base mismatches, provided that the effector sequence is capable of forming a duplex with a sequence set forth in SEQ ID NO:133; and (ii) an effector complement sequence comprising a sequence which is substantially complementary to the effector sequence. For example, shmiR-CD3-ε may comprise an effector sequence set forth in SEQ ID NO:132 and an effector complement sequence which is substantially complementary to the sequence set forth in SEQ ID NO:132 and capable of forming a duplex therewith. The effector complement sequence which is substantially complementary to the sequence set forth in SEQ ID NO:132 may be the sequence set forth in SEQ ID NO:133. A shmiR targeting the CD3-ε subunit in accordance with this example is hereinafter designated “shmiR-CD3-ε_2”.

In one example, shmiR-TCR-α as described herein comprises: (i) an effector sequence which is substantially complementary to the sequence set forth in SEQ ID NO:101, with the exception of 1, 2, 3 or 4 base mismatches, provided that the effector sequence is capable of forming a duplex with a sequence set forth in SEQ ID NO:101; and (ii) an effector complement sequence comprising a sequence which is substantially complementary to the effector sequence. For example, shmiR-TCR-α may comprise an effector sequence set forth in SEQ ID NO:100 and an effector complement sequence which is substantially complementary to the sequence set forth in SEQ ID NO:100 and capable of forming a duplex therewith. The effector complement sequence which is substantially complementary to the sequence set forth in SEQ ID NO:100 may be the sequence set forth in SEQ ID NO:101. A shmiR targeting the TCR-α subunit in accordance with this example is hereinafter designated “shmiR-TCR-α_1”.

In one example, shmiR-TCR-α as described herein comprises: (i) an effector sequence which is substantially complementary to the sequence set forth in SEQ ID NO:103, with the exception of 1, 2, 3 or 4 base mismatches, provided that the effector sequence is capable of forming a duplex with a sequence set forth in SEQ ID NO:103; and (ii) an effector complement sequence comprising a sequence which is substantially complementary to the effector sequence. For example, shmiR-TCR-α may comprise an effector sequence set forth in SEQ ID NO:102 and an effector complement sequence which is substantially complementary to the sequence set forth in SEQ ID NO:102 and capable of forming a duplex therewith. The effector complement sequence which is substantially complementary to the sequence set forth in SEQ ID NO:102 may be the sequence set forth in SEQ ID NO:103. A shmiR targeting the TCR-α subunit in accordance with this example is hereinafter designated “shmiR-TCR-α_2”.

In one example, shmiR-TCR-α as described herein comprises: (i) an effector sequence which is substantially complementary to the sequence set forth in SEQ ID NO:105, with the exception of 1, 2, 3 or 4 base mismatches, provided that the effector sequence is capable of forming a duplex with a sequence set forth in SEQ ID NO:105; and (ii) an effector complement sequence comprising a sequence which is substantially complementary to the effector sequence. For example, shmiR-TCR-α may comprise an effector sequence set forth in SEQ ID NO:104 and an effector complement sequence which is substantially complementary to the sequence set forth in SEQ ID NO:104 and capable of forming a duplex therewith. The effector complement sequence which is substantially complementary to the sequence set forth in SEQ ID NO:104 may be the sequence set forth in SEQ ID NO:105. A shmiR targeting the TCR-α subunit in accordance with this example is hereinafter designated “shmiR-TCR-α_3”.

In one example, shmiR-TCR-α as described herein comprises: (i) an effector sequence which is substantially complementary to the sequence set forth in SEQ ID NO:107, with the exception of 1, 2, 3 or 4 base mismatches, provided that the effector sequence is capable of forming a duplex with a sequence set forth in SEQ ID NO:107; and (ii) an effector complement sequence comprising a sequence which is substantially complementary to the effector sequence. For example, shmiR-TCR-α may comprise an effector sequence set forth in SEQ ID NO:106 and an effector complement sequence which is substantially complementary to the sequence set forth in SEQ ID NO:106 and capable of forming a duplex therewith. The effector complement sequence which is substantially complementary to the sequence set forth in SEQ ID NO:106 may be the sequence set forth in SEQ ID NO:107. A shmiR targeting the TCR-α subunit in accordance with this example is hereinafter designated “shmiR-TCR-α_4”.

In one example, shmiR-TCR-β as described herein comprises: (i) an effector sequence which is substantially complementary to the sequence set forth in SEQ ID NO:117 with the exception of 1, 2, 3 or 4 base mismatches, provided that the effector sequence is capable of forming a duplex with a sequence set forth in SEQ ID NO:117; and (ii) an effector complement sequence comprising a sequence which is substantially complementary to the effector sequence. For example, shmiR-TCR-β may comprise an effector sequence set forth in SEQ ID NO:116 and an effector complement sequence which is substantially complementary to the sequence set forth in SEQ ID NO:116 and capable of forming a duplex therewith. The effector complement sequence which is substantially complementary to the sequence set forth in SEQ ID NO:116 may be the sequence set forth in SEQ ID NO:117. A shmiR targeting the TCR-β subunit in accordance with this example is hereinafter designated “shmiR-TCR-β_5”.

In one example, shmiR-TCR-β as described herein comprises: (i) an effector sequence which is substantially complementary to the sequence set forth in SEQ ID NO:109 with the exception of 1, 2, 3 or 4 base mismatches, provided that the effector sequence is capable of forming a duplex with a sequence set forth in SEQ ID NO:109; and (ii) an effector complement sequence comprising a sequence which is substantially complementary to the effector sequence. For example, shmiR-TCR-β may comprise an effector sequence set forth in SEQ ID NO:108 and an effector complement sequence which is substantially complementary to the sequence set forth in SEQ ID NO:108 and capable of forming a duplex therewith. The effector complement sequence which is substantially complementary to the sequence set forth in SEQ ID NO:108 may be the sequence set forth in SEQ ID NO:109. A shmiR targeting the TCR-β subunit in accordance with this example is hereinafter designated “shmiR-TCR-β_1”.

In one example, shmiR-TCR-β as described herein comprises: (i) an effector sequence which is substantially complementary to the sequence set forth in SEQ ID NO:111 with the exception of 1, 2, 3 or 4 base mismatches, provided that the effector sequence is capable of forming a duplex with a sequence set forth in SEQ ID NO:111; and (ii) an effector complement sequence comprising a sequence which is substantially complementary to the effector sequence. For example, shmiR-TCR-β may comprise an effector sequence set forth in SEQ ID NO:110 and an effector complement sequence which is substantially complementary to the sequence set forth in SEQ ID NO:110 and capable of forming a duplex therewith. The effector complement sequence which is substantially complementary to the sequence set forth in SEQ ID NO:110 may be the sequence set forth in SEQ ID NO:111. A shmiR targeting the TCR-β subunit in accordance with this example is hereinafter designated “shmiR-TCR-β_2”.

In one example, shmiR-TCR-β as described herein comprises: (i) an effector sequence which is substantially complementary to the sequence set forth in SEQ ID NO:113 with the exception of 1, 2, 3 or 4 base mismatches, provided that the effector sequence is capable of forming a duplex with a sequence set forth in SEQ ID NO:113; and (ii) an effector complement sequence comprising a sequence which is substantially complementary to the effector sequence. For example, shmiR-TCR-β may comprise an effector sequence set forth in SEQ ID NO:112 and an effector complement sequence which is substantially complementary to the sequence set forth in SEQ ID NO:112 and capable of forming a duplex therewith. The effector complement sequence which is substantially complementary to the sequence set forth in SEQ ID NO:112 may be the sequence set forth in SEQ ID NO:113. A shmiR targeting the TCR-β subunit in accordance with this example is hereinafter designated “shmiR-TCR-β_3”.

In one example, shmiR-TCR-β as described herein comprises: (i) an effector sequence which is substantially complementary to the sequence set forth in SEQ ID NO:115 with the exception of 1, 2, 3 or 4 base mismatches, provided that the effector sequence is capable of forming a duplex with a sequence set forth in SEQ ID NO:115; and (ii) an effector complement sequence comprising a sequence which is substantially complementary to the effector sequence. For example, shmiR-TCR-β may comprise an effector sequence set forth in SEQ ID NO:114 and an effector complement sequence which is substantially complementary to the sequence set forth in SEQ ID NO:114 and capable of forming a duplex therewith. The effector complement sequence which is substantially complementary to the sequence set forth in SEQ ID NO:114 may be the sequence set forth in SEQ ID NO:115. A shmiR targeting the TCR-β subunit in accordance with this example is hereinafter designated “shmiR-TCR-β_4”.

In one example, shmiR-CD3-γ as described herein comprises: (i) an effector sequence which is substantially complementary to the sequence set forth in SEQ ID NO:121 with the exception of 1, 2, 3 or 4 base mismatches, provided that the effector sequence is capable of forming a duplex with a sequence set forth in SEQ ID NO: 121; and (ii) an effector complement sequence comprising a sequence which is substantially complementary to the effector sequence. For example, shmiR-CD3-γ may comprise an effector sequence set forth in SEQ ID NO:120 and an effector complement sequence which is substantially complementary to the sequence set forth in SEQ ID NO:120 and capable of forming a duplex therewith. The effector complement sequence which is substantially complementary to the sequence set forth in SEQ ID NO:120 may be the sequence set forth in SEQ ID NO:121. A shmiR targeting the CD3-γ subunit in accordance with this example is hereinafter designated “shmiR-CD3-γ_2”.

In one example, shmiR-CD3-γ as described herein comprises: (i) an effector sequence which is substantially complementary to the sequence set forth in SEQ ID NO:119 with the exception of 1, 2, 3 or 4 base mismatches, provided that the effector sequence is capable of forming a duplex with a sequence set forth in SEQ ID NO: 119; and (ii) an effector complement sequence comprising a sequence which is substantially complementary to the effector sequence. For example, shmiR-CD3-γ may comprise an effector sequence set forth in SEQ ID NO:118 and an effector complement sequence which is substantially complementary to the sequence set forth in SEQ ID NO:118 and capable of forming a duplex therewith. The effector complement sequence which is substantially complementary to the sequence set forth in SEQ ID NO:118 may be the sequence set forth in SEQ ID NO:119. A shmiR targeting the CD3-γ subunit in accordance with this example is hereinafter designated “shmiR-CD3-γ_1”.

In one example, shmiR-CD3-δ as described herein comprises: (i) an effector sequence which is substantially complementary to the sequence set forth in SEQ ID NO:127 with the exception of 1, 2, 3 or 4 base mismatches, provided that the effector sequence is capable of forming a duplex with a sequence set forth in SEQ ID NO:127; and (ii) an effector complement sequence comprising a sequence which is substantially complementary to the effector sequence. For example, shmiR-CD3-δ may comprise an effector sequence set forth in SEQ ID NO:126 and an effector complement sequence which is substantially complementary to the sequence set forth in SEQ ID NO:126 and capable of forming a duplex therewith. The effector complement sequence which is substantially complementary to the sequence set forth in SEQ ID NO:126 may be the sequence set forth in SEQ ID NO:127. A shmiR targeting the CD3-δ subunit in accordance with this example is hereinafter designated “shmiR-CD3-δ_3”.

In one example, shmiR-CD3-δ as described herein comprises: (i) an effector sequence which is substantially complementary to the sequence set forth in SEQ ID NO:123 with the exception of 1, 2, 3 or 4 base mismatches, provided that the effector sequence is capable of forming a duplex with a sequence set forth in SEQ ID NO:123; and (ii) an effector complement sequence comprising a sequence which is substantially complementary to the effector sequence. For example, shmiR-CD3-δ may comprise an effector sequence set forth in SEQ ID NO:122 and an effector complement sequence which is substantially complementary to the sequence set forth in SEQ ID NO:122 and capable of forming a duplex therewith. The effector complement sequence which is substantially complementary to the sequence set forth in SEQ ID NO:122 may be the sequence set forth in SEQ ID NO:123. A shmiR targeting the CD3-δ subunit in accordance with this example is hereinafter designated “shmiR-CD3-δ_1”.

In one example, shmiR-CD3-δ as described herein comprises: (i) an effector sequence which is substantially complementary to the sequence set forth in SEQ ID NO:125 with the exception of 1, 2, 3 or 4 base mismatches, provided that the effector sequence is capable of forming a duplex with a sequence set forth in SEQ ID NO:125; and (ii) an effector complement sequence comprising a sequence which is substantially complementary to the effector sequence. For example, shmiR-CD3-δ may comprise an effector sequence set forth in SEQ ID NO:124 and an effector complement sequence which is substantially complementary to the sequence set forth in SEQ ID NO:124 and capable of forming a duplex therewith. The effector complement sequence which is substantially complementary to the sequence set forth in SEQ ID NO:124 may be the sequence set forth in SEQ ID NO:125. A shmiR targeting the CD3-δ subunit in accordance with this example is hereinafter designated “shmiR-CD3-δ_2”.

In one example, shmiR-CD3-δ as described herein comprises: (i) an effector sequence which is substantially complementary to the sequence set forth in SEQ ID NO:129 with the exception of 1, 2, 3 or 4 base mismatches, provided that the effector sequence is capable of forming a duplex with a sequence set forth in SEQ ID NO:129; and (ii) an effector complement sequence comprising a sequence which is substantially complementary to the effector sequence. For example, shmiR-CD3-δ may comprise an effector sequence set forth in SEQ ID NO:128 and an effector complement sequence which is substantially complementary to the sequence set forth in SEQ ID NO:128 and capable of forming a duplex therewith. The effector complement sequence which is substantially complementary to the sequence set forth in SEQ ID NO:128 may be the sequence set forth in SEQ ID NO:129. A shmiR targeting the CD3-δ subunit in accordance with this example is hereinafter designated “shmiR-CD3-δ_4”.

In any of the examples described herein, the shmiRs comprise, in a 5′ to 3′ direction:

a 5′ flanking sequence of the pri-miRNA backbone;

the effector complement sequence;

the stemloop sequence;

the effector sequence; and

a 3′ flanking sequence of the pri-miRNA backbone.

Suitable loop sequences may be selected from those known in the art. However, an exemplary stemloop sequence is set forth in SEQ ID NO: 97.

Suitable primary micro RNA (pri-miRNA or pri-R) backbones for use in a nucleic acid of the disclosure may be selected from those known in the art. For example, the pri-miRNA backbone may be selected from a pri-miR-30a backbone, a pri-miR-155 backbone, a pri-miR-21 backbone and a pri-miR-136 backbone. For example, the pri-miRNA backbone is a pri-miR-30a backbone. In accordance with an example in which the pri-miRNA backbone is a pri-miR-30a backbone, the 5′ flanking sequence of the pri-miRNA backbone is set forth in SEQ ID NO: 98 and the 3′ flanking sequence of the pri-miRNA backbone is set forth in SEQ ID NO: 99. Thus, the nucleic acid encoding the respective shmiRs of the disclosure may comprise DNA sequence encoding the sequence set forth in SEQ ID NO: 98 and DNA sequence encoding the sequence set forth in SEQ ID NO: 99.

According to an example in which shmiR-CD3-ε comprises a pri-miR-30a backbone as described herein and a stemloop sequence set forth in SEQ ID NO: 97, shmiR-CD3-ε may comprise or consist of sequence set forth in one of SEQ ID NOs: 153, 151 or 152. Accordingly, a nucleic acid sequence coding for shmiR-CD3-ε may comprise or consist of the DNA sequence set forth in one of SEQ ID NOs: 171, 169 or 170, respectively. In one example, the shmiR targeting the CD3-ε subunit is shmiR-CD3-ε_3 comprising or consisting of the sequence set forth in SEQ ID NO: 153, which is encoded by the DNA sequence set forth in SEQ ID NO: 171. In one example, the shmiR targeting the CD3-ε subunit is shmiR-CD3-ε_1 comprising or consisting of the sequence set forth in SEQ ID NO: 151, which is encoded by the DNA sequence set forth in SEQ ID NO: 169. In one example, the shmiR targeting the CD3-ε subunit is shmiR-CD3-ε_2 comprising or consisting of the sequence set forth in SEQ ID NO: 152, which is encoded by the DNA sequence set forth in SEQ ID NO: 170.

According to an example in which shmiR-TCR-α comprises a pri-miR-30a backbone as described herein and a stemloop sequence set forth in SEQ ID NO:97, shmiR-TCR-α may comprise or consist of a sequence set forth in one of SEQ ID NOs: 136-139. Accordingly, a nucleic acid sequence coding for shmiR-TCR-α may comprise or consist of the DNA sequence set forth in one of SEQ ID NOs: 154-157, respectively. In one example, the shmiR targeting the TCR-α subunit is shmiR-TCR-α_1 comprising or consisting of the sequence set forth in SEQ ID NO: 136, which is encoded by the DNA sequence set forth in SEQ ID NO: 154. In one example, the shmiR targeting the TCR-α subunit is shmiR-TCR-α_2 comprising or consisting of the sequence set forth in SEQ ID NO: 137, which is encoded by the DNA sequence set forth in SEQ ID NO: 155. In one example, the shmiR targeting the TCR-α subunit is shmiR-TCR-α_3 comprising or consisting of the sequence set forth in SEQ ID NO: 138, which is encoded by the DNA sequence set forth in SEQ ID NO: 156. In one example, the shmiR targeting the TCR-α subunit is shmiR-TCR-α_4 comprising or consisting of the sequence set forth in SEQ ID NO: 139, which is encoded by the DNA sequence set forth in SEQ ID NO: 157.

According to an example in which shmiR-TCR-β comprises a pri-miR-30a backbone as described herein and a stemloop sequence set forth in SEQ ID NO: 97, shmiR-TCR-β may comprise or consist of sequence set forth in one of SEQ ID NOs: 144 or 140-143. Accordingly, a nucleic acid sequence coding for shmiR-TCR-α may comprise or consist of the DNA sequence set forth in one of SEQ ID NOs: 162 or 158-161, respectively. In one example, the shmiR targeting the TCR-β subunit is TCR-β subunit may be shmiR-TCR-β_5 comprising or consisting of the sequence set forth in SEQ ID NO: 144, which is encoded by the DNA sequence set forth in SEQ ID NO: 162. In one example, the shmiR targeting the TCR-β subunit is shmiR-TCR-β_1 comprising or consisting of the sequence set forth in SEQ ID NO: 140, which is encoded by the DNA sequence set forth in SEQ ID NO: 158. In one example, the shmiR targeting the TCR-β subunit is shmiR-TCR-β_2 comprising or consisting of the sequence set forth in SEQ ID NO: 141, which is encoded by the DNA sequence set forth in SEQ ID NO: 159. In one example, the shmiR targeting the TCR-β subunit is shmiR-TCR-β_3 comprising or consisting of the sequence set forth in SEQ ID NO: 142, which is encoded by the DNA sequence set forth in SEQ ID NO: 160. In one example, the shmiR targeting the TCR-β subunit is shmiR-TCR-β_4 comprising or consisting of the sequence set forth in SEQ ID NO: 143, which is encoded by the DNA sequence set forth in SEQ ID NO: 161.

According to an example in which shmiR-CD3-γ comprises a pri-miR-30a backbone as described herein and a stemloop sequence set forth in SEQ ID NO: 97, shmiR-CD3-γ may comprise or consist of sequence set forth in one of SEQ ID NOs: 146 or 145. Accordingly, a nucleic acid sequence coding for shmiR-CD3-γ may comprise or consist of the DNA sequence set forth in one of SEQ ID NOs: 164 or 163, respectively. In one example, the shmiR targeting the CD3-γ subunit is shmiR-CD3-γ_2 comprising or consisting of the sequence set forth in SEQ ID NO: 146, which is encoded by the DNA sequence set forth in SEQ ID NO: 164. In one example, the shmiR targeting the CD3-γ subunit is shmiR-CD3-γ_1 comprising or consisting of the sequence set forth in SEQ ID NO: 145, which is encoded by the DNA sequence set forth in SEQ ID NO: 163.

According to an example in which shmiR-CD3-δ comprises a pri-miR-30a backbone as described herein and a stemloop sequence set forth in SEQ ID NO: 97, shmiR-CD3-δ may comprise or consist of sequence set forth in one of SEQ ID NOs: 149, 147, 148 or 150. Accordingly, a nucleic acid sequence coding for shmiR-CD3-δ may comprise or consist of the DNA sequence set forth in one of SEQ ID NOs: 167, 165, 166 or 168, respectively. In one example, the shmiR targeting the CD3-δ subunit is shmiR-CD3-δ_3 comprising or consisting of the sequence set forth in SEQ ID NO: 149, which is encoded by the DNA sequence set forth in SEQ ID NO: 167. In one example, the shmiR targeting the CD3-δ subunit is shmiR-CD3-δ_1 comprising or consisting of the sequence set forth in SEQ ID NO: 147, which is encoded by the DNA sequence set forth in SEQ ID NO: 165. In one example, the shmiR targeting the CD3-δ subunit is shmiR-CD3-δ_2 comprising or consisting of the sequence set forth in SEQ ID NO: 148, which is encoded by the DNA sequence set forth in SEQ ID NO: 166. In one example, the shmiR targeting the CD3-δ subunit is shmiR-CD3-δ_4 comprising or consisting of the sequence set forth in SEQ ID NO: 150, which is encoded by the DNA sequence set forth in SEQ ID NO: 168.

As described herein, the ddRNAi construct of the disclosure comprises two or more nucleic acids with a DNA sequence coding for a shmiR targeting a subunit of the TCR complex. In some examples, the shmiRs encoded by the at least two nucleic acids may target different regions of the same mRNA transcript corresponding to a single TCR subunit. In other examples, the ddRNAi construct encodes at least two shmiRs comprising effector sequences which target mRNA transcripts of different subunits of the TCR complex. In this way, multiple subunits of the TCR complex may be targeted for RNAi by the ddRNAi construct.

In one example, the ddRNAi construct of the disclosure comprises two or more nucleic acids selected from:

(i) a nucleic acid comprising or consisting of a DNA sequence coding for shmiR-CD3-ε as described herein.
(ii) a nucleic acid comprising or consisting of a DNA sequence coding for shmiR-TCR-α as described herein;
(iii) a nucleic acid comprising or consisting of a DNA sequence coding for shmiR-TCR-β as described herein;
(iv) a nucleic acid comprising or consisting of a DNA sequence coding for shmiR-CD3-γ as described herein;
(v) a nucleic acid comprising or consisting of a DNA sequence coding for shmiR-CD3-δ as described herein.

In one example, the ddRNAi construct of the disclosure comprises a nucleic acid comprising or consisting of a DNA sequence coding for shmiR-CD3-ε as described herein, and one or more further nucleic acids which comprise or consist of a DNA sequence coding for a shmiR selected from shmiR-TCR-α, shmiR-TCR-β, shmiR-CD3-γ and shmiR-CD3-δ each of which are as described herein.

In one example, the ddRNAi construct of the disclosure comprises a nucleic acid comprising or consisting of a DNA sequence coding for shmiR-TCR-α as described herein, and one or more further nucleic acids which comprise or consist of a DNA sequence coding for a shmiR selected from shmiR-TCR-β, shmiR-CD3-γ, shmiR-CD3-δ and shmiR-CD3-ε each of which are as described herein.

In one example, the ddRNAi construct of the disclosure comprises a nucleic acid comprising or consisting of a DNA sequence coding for shmiR-TCR-β as described herein, and one or more further nucleic acids which comprise or consist of a DNA sequence coding for a shmiR selected from shmiR-TCR-α, shmiR-CD3-γ, shmiR-CD3-δ and shmiR-CD3-ε each of which are as described herein.

In one example, the ddRNAi construct of the disclosure comprises a nucleic acid comprising or consisting of a DNA sequence coding for shmiR-CD3-γ as described herein, and one or more further nucleic acids which comprise or consist of a DNA sequence coding for a shmiR selected from shmiR-TCR-α, shmiR-TCR-β, shmiR-CD3-δ and shmiR-CD3-ε each of which are as described herein.

In one example, the ddRNAi construct of the disclosure comprises a nucleic acid comprising or consisting of a DNA sequence coding for shmiR-CD3-δ as described herein, and one or more further nucleic acids which comprise or consist of a DNA sequence coding for a shmiR selected from shmiR-TCR-α, shmiR-TCR-β, shmiR-CD3-γ and shmiR-CD3-ε each of which are as described herein.

In one example, the ddRNAi construct of the disclosure comprises two or more nucleic acids selected from:

(i) a nucleic acid comprising or consisting of a DNA sequence coding for shmiR-TCR-β as described herein;
(ii) a nucleic acid comprising or consisting of a DNA sequence coding for shmiR-CD3-γ as described herein; and
(iii) a nucleic acid comprising or consisting of a DNA sequence coding for shmiR-CD3-ε as described herein.

For example, the ddRNAi construct of the disclosure may comprise:

(i) a nucleic acid comprising or consisting of a DNA sequence coding for shmiR-TCR-β as described herein;
(ii) a nucleic acid comprising or consisting of a DNA sequence coding for shmiR-CD3-γ as described herein; and
(iii) a nucleic acid comprising or consisting of a DNA sequence coding for shmiR-CD3-ε as described herein.

In one example, the ddRNAi construct of the disclosure comprises two or more nucleic acids selected from:

(i) a nucleic acid comprising or consisting of a DNA sequence coding for shmiR-TCR-α as described herein;
(ii) a nucleic acid comprising or consisting of a DNA sequence coding for shmiR-TCR-β as described herein; and
(iii) a nucleic acid comprising or consisting of a DNA sequence coding for shmiR-CD3-ε as described herein.

For example, the ddRNAi construct of the disclosure may comprise:

(i) a nucleic acid comprising or consisting of a DNA sequence coding for shmiR-TCR-α as described herein;
(ii) a nucleic acid comprising or consisting of a DNA sequence coding for shmiR-TCR-β as described herein; and
(iii) a nucleic acid comprising or consisting of a DNA sequence coding for shmiR-CD3-ε as described herein.

In one example, the ddRNAi construct of the disclosure comprises two or more nucleic acids selected from:

(i) a nucleic acid comprising or consisting of a DNA sequence coding for shmiR-TCR-α as described herein;
(ii) a nucleic acid comprising or consisting of a DNA sequence coding for shmiR-CD3-γ as described herein; and
(iii) a nucleic acid comprising or consisting of a DNA sequence coding for shmiR-CD3-ε as described herein.

For example, the ddRNAi construct of the disclosure may comprise:

(i) a nucleic acid comprising or consisting of a DNA sequence coding for shmiR-TCR-α as described herein;
(ii) a nucleic acid comprising or consisting of a DNA sequence coding for shmiR-CD3-γ as described herein; and
(iii) a nucleic acid comprising or consisting of a DNA sequence coding for shmiR-CD3-ε as described herein.

In one example, the ddRNAi construct of the disclosure comprises two or more nucleic acids selected from:

(i) a nucleic acid comprising or consisting of a DNA sequence coding for shmiR-TCR-α as described herein;
(ii) a nucleic acid comprising or consisting of a DNA sequence coding for shmiR-CD3-δ as described herein; and
(iii) a nucleic acid comprising or consisting of a DNA sequence coding for shmiR-CD3-ε as described herein.

For example, the ddRNAi construct of the disclosure may comprise:

(i) a nucleic acid comprising or consisting of a DNA sequence coding for shmiR-TCR-α as described herein;
(ii) a nucleic acid comprising or consisting of a DNA sequence coding for shmiR-CD3-δ as described herein; and
(iii) a nucleic acid comprising or consisting of a DNA sequence coding for shmiR-CD3-ε as described herein.

ShmiRs designated shmiR-TCR-α, shmiR-TCR-β, shmiR-TCR-γ, shmiR-TCR-δ and shmiR-CD3-ε, including DNA sequences coding for same, have been described herein and shall be taken to apply mutatis mutandis to each example of the disclosure.

In one example, the at least two nucleic acids are selected from the group consisting of:

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3);

a nucleic acid comprising or consisting of a DNA sequence encoding a shmiR comprising an effector sequence set forth in SEQ ID NO: 100 and an effector complement sequence set forth in SEQ ID NO: 101 e.g., a nucleic acid comprising or consisting of a DNA sequence set forth in SEQ ID NO: 154 and encoding a shmiR with a sequence set forth in SEQ ID NO:136 (shmiR-TCR-α_1);

a nucleic acid comprising or consisting of a DNA sequence encoding a shmiR comprising an effector sequence set forth in SEQ ID NO: 116 and an effector complement sequence set forth in SEQ ID NO: 117 e.g., a nucleic acid comprising or consisting of a DNA sequence set forth in SEQ ID NO: 162 and encoding a shmiR with a sequence set forth in SEQ ID NO:144 (shmiR-TCR-β_5);

a nucleic acid comprising or consisting of a DNA sequence encoding a shmiR comprising an effector sequence set forth in SEQ ID NO: 120 and an effector complement sequence set forth in SEQ ID NO: 121 e.g., a nucleic acid comprising or consisting of a DNA sequence set forth in SEQ ID NO: 164 and encoding a shmiR with a sequence set forth in SEQ ID NO:146 (shmiR-CD3-γ_2); and a nucleic acid comprising or consisting of a DNA sequence encoding a shmiR comprising an effector sequence set forth in SEQ ID NO: 126 and an effector complement sequence set forth in SEQ ID NO: 127 e.g., a nucleic acid comprising or consisting of a DNA sequence set forth in SEQ ID NO: 167 and encoding a shmiR with a sequence set forth in SEQ ID NO:149 (shmiR-CD3-δ_3).

e.g., In one example, the ddRNAi construct comprises at least two nucleic acids selected from the group consisting of:

a nucleic acid comprising or consisting of a DNA sequence encoding a shmiR comprising an effector sequence set forth in SEQ ID NO: 116 and an effector complement sequence set forth in SEQ ID NO: 117 e.g., a nucleic acid comprising or consisting of a DNA sequence set forth in SEQ ID NO: 162 and encoding a shmiR with a sequence set forth in SEQ ID NO:144 (shmiR-TCR-β_5);

a nucleic acid comprising or consisting of a DNA sequence encoding a shmiR comprising an effector sequence set forth in SEQ ID NO: 120 and an effector complement sequence set forth in SEQ ID NO: 121 e.g., a nucleic acid comprising or consisting of a DNA sequence set forth in SEQ ID NO: 164 and encoding a shmiR with a sequence set forth in SEQ ID NO:146 (shmiR-CD3-γ_2); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In one example, the ddRNAi construct comprises:

a nucleic acid comprising or consisting of a DNA sequence encoding a shmiR comprising an effector sequence set forth in SEQ ID NO: 116 and an effector complement sequence set forth in SEQ ID NO: 117 e.g., a nucleic acid comprising or consisting of a DNA sequence set forth in SEQ ID NO: 162 and encoding a shmiR with a sequence set forth in SEQ ID NO:144 (shmiR-TCR-β_5);

a nucleic acid comprising or consisting of a DNA sequence encoding a shmiR comprising an effector sequence set forth in SEQ ID NO: 120 and an effector complement sequence set forth in SEQ ID NO: 121 e.g., a nucleic acid comprising or consisting of a DNA sequence set forth in SEQ ID NO: 164 and encoding a shmiR with a sequence set forth in SEQ ID NO:146 (shmiR-CD3-γ_2); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In one example, the ddRNAi construct comprises at least two nucleic acids selected from the group consisting of:

a nucleic acid comprising or consisting of a DNA sequence encoding a shmiR comprising an effector sequence set forth in SEQ ID NO: 100 and an effector complement sequence set forth in SEQ ID NO: 101 e.g., a nucleic acid comprising or consisting of a DNA sequence set forth in SEQ ID NO: 154 and encoding a shmiR with a sequence set forth in SEQ ID NO:136 (shmiR-TCR-α_1);

a nucleic acid comprising or consisting of a DNA sequence encoding a shmiR comprising an effector sequence set forth in SEQ ID NO: 116 and an effector complement sequence set forth in SEQ ID NO: 117 e.g., a nucleic acid comprising or consisting of a DNA sequence set forth in SEQ ID NO: 162 and encoding a shmiR with a sequence set forth in SEQ ID NO:144 (shmiR-TCR-β_5); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In one example, the ddRNAi construct comprises:

a nucleic acid comprising or consisting of a DNA sequence encoding a shmiR comprising an effector sequence set forth in SEQ ID NO: 100 and an effector complement sequence set forth in SEQ ID NO: 101 e.g., a nucleic acid comprising or consisting of a DNA sequence set forth in SEQ ID NO: 154 and encoding a shmiR with a sequence set forth in SEQ ID NO:136 (shmiR-TCR-α_1);

a nucleic acid comprising or consisting of a DNA sequence encoding a shmiR comprising an effector sequence set forth in SEQ ID NO: 116 and an effector complement sequence set forth in SEQ ID NO: 117 e.g., a nucleic acid comprising or consisting of a DNA sequence set forth in SEQ ID NO: 162 and encoding a shmiR with a sequence set forth in SEQ ID NO:144 (shmiR-TCR-β_5); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In one example, the ddRNAi construct comprises at least two nucleic acids selected from the group consisting of:

a nucleic acid comprising or consisting of a DNA sequence encoding a shmiR comprising an effector sequence set forth in SEQ ID NO: 100 and an effector complement sequence set forth in SEQ ID NO: 101 e.g., a nucleic acid comprising or consisting of a DNA sequence set forth in SEQ ID NO: 154 and encoding a shmiR with a sequence set forth in SEQ ID NO:136 (shmiR-TCR-α_1);

a nucleic acid comprising or consisting of a DNA sequence encoding a shmiR comprising an effector sequence set forth in SEQ ID NO: 120 and an effector complement sequence set forth in SEQ ID NO: 121 e.g., a nucleic acid comprising or consisting of a DNA sequence set forth in SEQ ID NO: 164 and encoding a shmiR with a sequence set forth in SEQ ID NO:146 (shmiR-CD3-γ_2); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In one example, the ddRNAi construct comprises:

a nucleic acid comprising or consisting of a DNA sequence encoding a shmiR comprising an effector sequence set forth in SEQ ID NO: 100 and an effector complement sequence set forth in SEQ ID NO: 101 e.g., a nucleic acid comprising or consisting of a DNA sequence set forth in SEQ ID NO: 154 and encoding a shmiR with a sequence set forth in SEQ ID NO:136 (shmiR-TCR-α_1);

a nucleic acid comprising or consisting of a DNA sequence encoding a shmiR comprising an effector sequence set forth in SEQ ID NO: 120 and an effector complement sequence set forth in SEQ ID NO: 121 e.g., a nucleic acid comprising or consisting of a DNA sequence set forth in SEQ ID NO: 164 and encoding a shmiR with a sequence set forth in SEQ ID NO:146 (shmiR-CD3-γ_2); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In one example, the ddRNAi construct comprises at least two nucleic acids selected from the group consisting of:

a nucleic acid comprising or consisting of a DNA sequence encoding a shmiR comprising an effector sequence set forth in SEQ ID NO: 100 and an effector complement sequence set forth in SEQ ID NO: 101 e.g., a nucleic acid comprising or consisting of a DNA sequence set forth in SEQ ID NO: 154 and encoding a shmiR with a sequence set forth in SEQ ID NO:136 (shmiR-TCR-α_1);

a nucleic acid comprising or consisting of a DNA sequence encoding a shmiR comprising an effector sequence set forth in SEQ ID NO: 126 and an effector complement sequence set forth in SEQ ID NO: 127 e.g., a nucleic acid comprising or consisting of a DNA sequence set forth in SEQ ID NO: 167 and encoding a shmiR with a sequence set forth in SEQ ID NO:149 (shmiR-CD3-δ_3); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In one example, the ddRNAi construct comprises:

a nucleic acid comprising or consisting of a DNA sequence encoding a shmiR comprising an effector sequence set forth in SEQ ID NO: 100 and an effector complement sequence set forth in SEQ ID NO: 101 e.g., a nucleic acid comprising or consisting of a DNA sequence set forth in SEQ ID NO: 154 and encoding a shmiR with a sequence set forth in SEQ ID NO:136 (shmiR-TCR-α_1);

a nucleic acid comprising or consisting of a DNA sequence encoding a shmiR comprising an effector sequence set forth in SEQ ID NO: 126 and an effector complement sequence set forth in SEQ ID NO: 127 e.g., a nucleic acid comprising or consisting of a DNA sequence set forth in SEQ ID NO: 167 and encoding a shmiR with a sequence set forth in SEQ ID NO:149 (shmiR-CD3-δ_3); and

a nucleic acid comprising or consisting of a DNA sequence encoding a shmiR comprising an effector sequence set forth in SEQ ID NO: 134 and an effector complement sequence set forth in SEQ ID NO: 135 e.g., a nucleic acid comprising or consisting of a DNA sequence set forth in SEQ ID NO: 171 and encoding a shmiR with a sequence set forth in SEQ ID NO:153 (shmiR-CD3-ε_3).

In accordance with any example of a ddRNAi construct as described herein, the ddRNAi construct may comprise two or more nucleic acids encoding shmiRs described herein, such as two, or three, or four, or five nucleic acids encoding shmiRs as described herein.

In some examples, a ddRNAi construct of the disclosure comprises a transcriptional terminator linked to one or more of the nucleic acids encoding a shmiR of the disclosure. The terminators linked to each nucleic acid encoding a shmiR can be the same or different. For example, in a ddRNAi construct of the disclosure in which a RNA pol III promoter is employed, the terminator may be a contiguous stretch of 4 or more or 5 or more or 6 or more T residues. However, where different promoters are used, the terminators can be different and are matched to the promoter from the gene from which the terminator is derived. Such terminators include, but are not limited to, the SV40 poly A, the AdV VA1 gene, the 5S ribosomal RNA gene, and the terminators for human t-RNAs.

Alternatively, or in addition, the nucleic acids comprised within the ddRNAi construct of the disclosure may comprise one or more restriction sites e.g., to facilitate cloning of the nucleic acid(s) into cloning or expression vectors. For example, the nucleic acids described herein may include a restriction site upstream and/or downstream of the DNA sequence encoding a shmiR of the disclosure. Suitable restriction enzyme recognition sequences will be known to a person of skill in the art. However, in one example, the nucleic acid(s) of the disclosure may include a BamH1 restriction site (GGATCC) at the 5′ terminus i.e., upstream of the sequence encoding the shmiR, and a HindIII restriction site (AAGCTT) at the 3′ terminus i.e., downstream of the DNA sequence encoding the shmiR.

In some examples, a ddRNAi construct of the disclosure may comprise a stuffer sequence to optimize construct or vector size. Suitable stuffer sequences for use in the construction of expression constructs and vectors are known in the art and contemplated for use herein. In one example, the ddRNAi construct of the disclosure includes a hypoxanthine-guanine phosphoribosyltransferase (HPRT) stuffer sequence. In each of the foregoing examples describing a ddRNAi construct of the disclosure, each nucleic acid comprised therein may be operably-linked to a promoter. For example, the ddRNAi construct as described herein may comprise a single promoter which is operably-linked to each nucleic acid comprised therein e.g., to drive expression of the two or more shmiRs from the ddRNAi construct.

In another example, each nucleic acid encoding a shmiR of the disclosure comprised in the ddRNAi construct is operably-linked to a separate promoter.

According to an example in which multiple promoters are present, the promoters can be the same or different. For example, the construct may comprise multiple copies of the same promoter with each copy operably-linked to a different nucleic acid of the disclosure. In another example, each promoter operably-linked to a nucleic acid of the disclosure is different. For example, the at least two nucleic acids in the ddRNAi construct encoding shmiRs may each be operably-linked to a different promoter.

In one example, the promoter is a constitutive promoter. The term “constitutive” when made in reference to a promoter means that the promoter is capable of directing transcription of an operably-linked nucleic acid sequence in the absence of a specific stimulus (e.g., heat shock, chemicals, light, etc.). Typically, constitutive promoters are capable of directing expression of a coding sequence in substantially any cell and any tissue. The promoters used to transcribe shmiRs from the nucleic acid(s) of the disclosure include promoters for ubiquitin, CMV, β-actin, histone H4, EF-1α or pgk genes controlled by RNA polymerase II, or promoter elements controlled by RNA polymerase I.

In one example, a Pol II promoter such as CMV, SV40, U1, β-actin or a hybrid Pol II promoter is employed. Other suitable Pol II promoters are known in the art and may be used in accordance with this example of the disclosure. For example, a Pol II promoter system may be desirable in a ddRNAi construct of the disclosure which expresses a pri-miRNA which, by the action of the enzymes Drosha and Pasha, is processed into one or more shmiRs. A Pol II promoter system may also be desirable in a ddRNAi construct of the disclosure comprising sequence encoding a plurality of shmiRs under control of a single promoter. A Pol II promoter system may also be used where tissue specificity is desired.

In another example, a promoter controlled by RNA polymerase III is used, such as a U6 promoter (U6-1, U6-8, U6-9), H1 promoter, 7SL promoter, a human Y promoter (hY1, hY3, hY4 (see Martha, et al., Nucleic Acids Res 22(15):3045-52(1994)) and hY5 (see Maraia, et al., Nucleic Acids Res 24(18):3552-59(1994)), a human MRP-7-2 promoter, an Adenovirus VA1 promoter, a human tRNA promoter, or a 5 s ribosomal RNA promoter.

Suitable promoters for use in a ddRNAi construct of the disclosure are described in U.S. Pat. Nos. 8,008,468 and 8,129,510.

In one example, the promoter is a RNA pol III promoter. For example, the promoter is a U6 promoter (e.g., a U6-1, U6-8 or U6-9 promoter). In another example, the promoter is a H1 promoter.

In the case of a ddRNAi construct of the disclosure as described herein, each of the nucleic acids in the ddRNAi construct may be operably linked to a U6 promoter e.g., a separate U6 promoter.

In one example, the promoter in a construct is a U6 promoter. For example, the promoter is a U6-1 promoter. For example, the promoter is a U6-8 promoter. For example, the promoter is a U6-9 promoter.

In one example, the construct comprises at least one U6 promoter and at least one H1 promoter, each operably linked to a separate DNA encoding a shmiR of the disclosure. For example, the U6 promoter may be a U6-1 promoter. For example, the U6 promoter may be a U6-8 promoter. For example, the U6 promoter may be a U6-9 promoter.

In some examples, promoters of variable strength are employed. For example, use of two or more strong promoters (such as a Pol III-type promoter) may tax the cell, by, e.g., depleting the pool of available nucleotides or other cellular components needed for transcription. In addition, or alternatively, use of several strong promoters may cause a toxic level of expression of shmiRs in the cell. Thus, in some examples one or more of the promoters in the multiple-promoter ddRNAi construct is weaker than other promoters in the construct, or all promoters in the construct may express the shmiRs at less than a maximum rate. Promoters may also be modified using various molecular techniques, or otherwise, e.g., through modification of various regulatory elements, to attain weaker levels or stronger levels of transcription. One means of achieving reduced transcription is to modify sequence elements within promoters known to control promoter activity. For example the Proximal Sequence Element (PSE) is known to effect the activity of human U6 promoters (see Domitrovich, et al., Nucleic Acids Res 31: 2344-2352 (2003). Replacing the PSE elements present in strong promoters, such as the human U6-1, U6-8 or U6-9 promoters, with the element from a weak promoter, such as the human U6-7 promoter, reduces the activity of the hybrid U6-1, U6-8 or U6-9 promoters. This approach has been used in the examples described in this application, but other means to achieve this outcome are known in the art.

Promoters useful in some examples of the present disclosure can be tissue-specific or cell-specific. The term “tissue specific” as it applies to a promoter refers to a promoter that is capable of directing selective transcription of a nucleic acid of interest to a specific type of tissue in the relative absence of expression of the same nucleotide sequence of interest in a different type of tissue. The term “cell-specific” as applied to a promoter refers to a promoter which is capable of directing selective transcription of a nucleic acid of interest in a specific type of cell in the relative absence of expression of the same nucleotide sequence of interest in a different type of cell within the same tissue.

In one example, a ddRNAi construct of the disclosure may additionally comprise one or more enhancers to increase expression of the shmiRs encoded by the nucleic acids described herein. Enhancers appropriate for use in examples of the present disclosure include the Apo E HCR enhancer, a CMV enhancer (Xia et al, Nucleic Acids Res 31-17(2003)), and other enhancers known to those skilled in the art. Suitable enhancers for use in a ddRNAi construct of the disclosure are described in U.S. Pat. No. 8,008,468.

In a further example, a ddRNAi construct of the disclosure may comprise a transcriptional terminator linked to a nucleic acid encoding a shmiR of the disclosure. The terminators linked to each nucleic acid in the ddRNAi construct can be the same or different. For example, in a ddRNAi construct of the disclosure in which a RNA pol III promoter is employed, the terminator may be a contiguous stretch of 4 or more or 5 or more or 6 or more T residues. However, where different promoters are used, the terminators can be different and are matched to the promoter from the gene from which the terminator is derived. Such terminators include, bit are not limited to, the SV40 poly A, the AdV VA1 gene, the 5S ribosomal RNA gene, and the terminators for human t-RNAs. Other promoter and terminator combinations are known in the art and are contemplated for use in a ddRNAi construct of the disclosure.

In addition, promoters and terminators may be mixed and matched, as is commonly done with RNA pol II promoters and terminators.

In one example, the promoter and terminator combinations used for each nucleic acid in a ddRNAi construct may be different to decrease the likelihood of DNA recombination events between components.

One exemplary ddRNAi construct of the disclosure comprises (i) a nucleic acid comprising or consisting of a DNA sequence encoding shmiR-TCR-β_5 as described herein operably-linked to a promoter e.g., a U6 promoter, and a transcription terminator sequence e.g., TTTTT (ii) a nucleic acid comprising or consisting of a DNA sequence encoding shmiR-CD3-γ_2 as described herein operably-linked to a promoter e.g., a U6 promoter, and a transcription terminator sequence e.g., TTTTT, (iii) and a nucleic acid comprising or consisting of a DNA sequence encoding shmiR-CD3-ε_3 as described herein operably-linked to a promoter e.g., a U6 or H1 promoter, and a transcription terminator sequence e.g., TTTTT. The U6 promoters may be selected from a U6-1, U6-8 and U6-9 promoter. For example, an exemplary ddRNAi construct of the disclosure comprises (i) a nucleic acid comprising or consisting of a DNA sequence set forth in SEQ ID NO:162 (shmiR-TCR-β_5) operably-linked to a U6 promoter e.g., a U6-9 promoter, and the transcription terminator sequence TTTTT (ii) a nucleic acid comprising or consisting of a DNA sequence set forth in SEQ ID NO:164 (shmiR-CD3-γ_2) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a U6 promoter e.g., a U6-8 promoter, and the transcription terminator sequence TTTTT. For example, a ddRNAi construct coding for shmiRs designated shmiR-TCR-β_5, shmiR-CD3-γ_2 and shmiR-CD3-ε_3 may comprise or consist of a DNA sequence set forth in SEQ ID NO: 175. An exemplary ddRNAi construct of the disclosure comprises (i) a nucleic acid comprising or consisting of a DNA sequence set forth in SEQ ID NO:162 (shmiR-TCR-β_5) operably-linked to a U6 promoter e.g., a U6-9 promoter, and the transcription terminator sequence TTTTT (ii) a nucleic acid comprising or consisting of a DNA sequence set forth in SEQ ID NO:164 (shmiR-CD3-γ_2) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a H1 promoter and the transcription terminator sequence TTTTT. For example, a ddRNAi construct coding for shmiRs designated shmiR-TCR-β_5, shmiR-CD3-γ_2 and shmiR-CD3-ε_3 may comprise or consist of a DNA sequence set forth in SEQ ID NO: 178.

Another exemplary ddRNAi construct of the disclosure comprises (i) a nucleic acid comprising or consisting of a DNA sequence encoding shmiR-TCR-α_1 as described herein operably-linked to a promoter e.g., a U6 promoter, and a transcription terminator sequence e.g., TTTTT (ii) a nucleic acid comprising or consisting of a DNA sequence encoding shmiR-TCR-β_5 as described herein operably-linked to a promoter e.g., a U6 promoter, and a transcription terminator sequence e.g., TTTTT, (iii) and a nucleic acid comprising or consisting of a DNA sequence encoding shmiR-CD3-ε_3 as described herein operably-linked to a promoter e.g., a U6 or H1 promoter, and a transcription terminator sequence e.g., TTTTT. The U6 promoters may be selected from a U6-1, U6-8 and U6-9 promoter. For example, an exemplary ddRNAi construct of the disclosure comprises (i) a nucleic acid comprising or consisting of a DNA sequence set forth in SEQ ID NO:154 (shmiR-TCR-α_1) operably-linked to a U6 promoter e.g., a U6-9 promoter, and the transcription terminator sequence TTTTT (ii) a nucleic acid comprising or consisting of a DNA sequence set forth in SEQ ID NO:162 (shmiR-TCR-β_5) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a U6 promoter e.g., a U6-8 promoter, and the transcription terminator sequence TTTTT. For example, a ddRNAi construct coding for shmiRs designated shmiR-TCR-α_1, shmiR-TCR-β_5 and shmiR-CD3-ε_3 may comprise or consist of a DNA sequence set forth in SEQ ID NO: 172. Another exemplary ddRNAi construct of the disclosure comprises (i) a nucleic acid comprising or consisting of a DNA sequence set forth in SEQ ID NO:154 (shmiR-TCR-α_1) operably-linked to a U6 promoter e.g., a U6-9 promoter, and the transcription terminator sequence TTTTT (ii) a nucleic acid comprising or consisting of a DNA sequence set forth in SEQ ID NO:162 (shmiR-TCR-β_5) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a H1 promoter and the transcription terminator sequence TTTTT. For example, a ddRNAi construct coding for shmiRs designated shmiR-TCR-α_1, shmiR-TCR-β_5 and shmiR-CD3-ε_3 may comprise or consist of a DNA sequence set forth in SEQ ID NO: 176.

Another exemplary ddRNAi construct of the disclosure comprises (i) a nucleic acid comprising or consisting of a DNA sequence encoding shmiR-TCR-α_1 as described herein operably-linked to a promoter e.g., a U6 promoter, and a transcription terminator sequence e.g., TTTTT (ii) a nucleic acid comprising or consisting of a DNA sequence encoding shmiR-CD3-γ_2 as described herein operably-linked to a promoter e.g., a U6 promoter, and a transcription terminator sequence e.g., TTTTT, (iii) and a nucleic acid comprising or consisting of a DNA sequence encoding shmiR-CD3-ε_3 as described herein operably-linked to a promoter e.g., a U6 or H1 promoter, and a transcription terminator sequence e.g., TTTTT. The U6 promoters may be selected from a U6-1, U6-8 and U6-9 promoter. For example, an exemplary ddRNAi construct of the disclosure comprises (i) a nucleic acid comprising or consisting of a DNA sequence set forth in SEQ ID NO:154 (shmiR-TCR-α_1) operably-linked to a U6 promoter e.g., a U6-9 promoter, and the transcription terminator sequence TTTTT (ii) a nucleic acid comprising or consisting of a DNA sequence set forth in SEQ ID NO:164 (shmiR-CD3-γ_2) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a U6 promoter e.g., a U6-8 promoter, and the transcription terminator sequence TTTTT. For example, a ddRNAi construct coding for shmiRs designated shmiR-TCR-α_1, shmiR-CD3-γ_2 and shmiR-CD3-ε_3 may comprise or consist of a DNA sequence set forth in SEQ ID NO: 173. Another exemplary ddRNAi construct of the disclosure comprises (i) a nucleic acid comprising or consisting of a DNA sequence set forth in SEQ ID NO:154 (shmiR-TCR-α_1) operably-linked to a U6 promoter e.g., a U6-9 promoter, and the transcription terminator sequence TTTTT (ii) a nucleic acid comprising or consisting of a DNA sequence set forth in SEQ ID NO:164 (shmiR-CD3-γ_2) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a H1 promoter and the transcription terminator sequence TTTTT. For example, a ddRNAi construct coding for shmiRs designated shmiR-TCR-α_1, shmiR-CD3-γ_2 and shmiR-CD3-ε_3 may comprise or consist of a DNA sequence set forth in SEQ ID NO: 177.

Another exemplary ddRNAi construct of the disclosure comprises (i) a nucleic acid comprising or consisting of a DNA sequence encoding shmiR-TCR-α_1 as described herein operably-linked to a promoter e.g., a U6 promoter, and a transcription terminator sequence e.g., TTTTT (ii) a nucleic acid comprising or consisting of a DNA sequence encoding shmiR-CD3-δ_3 as described herein operably-linked to a promoter e.g., a U6 promoter, and a transcription terminator sequence e.g., TTTTT, (iii) and a nucleic acid comprising or consisting of a DNA sequence encoding shmiR-CD3-ε_3 as described herein operably-linked to a promoter e.g., a U6 promoter, and a transcription terminator sequence e.g., TTTTT. The U6 promoters may be selected from a U6-1, U6-8 and U6-9 promoter. For example, an exemplary ddRNAi construct of the disclosure comprises (i) a nucleic acid comprising or consisting of a DNA sequence set forth in SEQ ID NO:154 (shmiR-TCR-α_1) operably-linked to a U6 promoter e.g., a U6-9 promoter, and the transcription terminator sequence TTTTT (ii) a nucleic acid comprising or consisting of a DNA sequence set forth in SEQ ID NO:167 (shmiR-CD3-δ_3) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a U6 promoter e.g., a U6-8 promoter, and the transcription terminator sequence TTTTT. For example, a ddRNAi construct coding for shmiRs designated shmiR-TCR-α_1, shmiR-CD3-δ_3 and shmiR-CD3-ε_3 may comprise or consist of a DNA sequence set forth in SEQ ID NO: 174.

In addition, the ddRNAi construct can comprise one or more multiple cloning sites and/or unique restriction sites that are located strategically, such that the promoters, nucleic acids encoding the shmiRs and/or other regulatory elements are easily removed or replaced. The ddRNAi construct can be assembled from smaller oligonucleotide components using strategically located restriction sites and/or complementary sticky ends. The base vector for one approach according to the present disclosure comprises plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into a suitable vector backbone using two unique restriction enzyme sites (the same or different ones) that flank the double-, triple- or multiple-expression cassette insert.

Generation of the ddRNAi construct can be accomplished using any suitable genetic engineering techniques known in the art, including without limitation, the standard techniques of PCR, oligonucleotide synthesis, restriction endonuclease digestion, ligation, transformation, plasmid purification, and DNA sequencing. If the construct is a viral construct, the construct comprises, for example, sequences necessary to package the ddRNAi construct into viral particles and/or sequences that allow integration of the ddRNAi construct into the target cell genome. In some examples, the viral construct additionally contains genes that allow for replication and propagation of virus, however such genes will be supplied in trans. Additionally, the viral construct can contain genes or genetic sequences from the genome of any known organism incorporated in native form or modified. For example, a viral construct may comprise sequences useful for replication of the construct in bacteria.

The ddRNAi construct also may contain additional genetic elements. The types of elements that may be included in the construct are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines.

Other genetic elements that may find use in embodiments of the present disclosure include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the construct, an internal ribosomal entry site (IRES) sequence can be included. In one example, the additional genetic elements are operably-linked with and controlled by an independent promoter/enhancer. In addition a suitable origin of replication for propagation of the construct in bacteria may be employed. The sequence of the origin of replication generally is separated from the ddRNAi construct and other genetic sequences. Such origins of replication are known in the art and include the pUC, ColE1, 2-micron or SV40 origins of replication.

### Chimeric Antigen Receptors (CAR)

The present disclosure also provides a chimeric antigen receptor (CAR) construct comprising a nucleic acid with a DNA sequence coding for a CAR.

In one example, the CAR construct is provided in a recombinant DNA construct with the ddRNAi construct of the disclosure. Accordingly, the present disclosure provide a DNA construct comprising:

(a) a ddRNAi construct as described herein; and
(b) a CAR construct comprising nucleic acid with a DNA sequence coding for a CAR.

In one example, the CAR comprises an antigen binding domain e.g., a binding protein.

In one example, the CAR may be an antibody or an antigen binding domain thereof.

In one example, the antigen binding domain binds specifically to a tumor antigen e.g., as described herein. In another example, the antigen binding domain binds specifically to a virus antigen or viral-induced antigen found on the surface of an infected cell e.g., such as antigen from a virus described herein.

In one example, the DNA sequence coding for the CAR is operably-linked to a promoter comprised within the CAR construct and positioned upstream of the DNA sequence coding the CAR. The promoter may be any suitable promoter known in the art for directing expression of a CAR e.g., an EF1p promoter element.

In accordance with an example in which the CAR construct is provided in a recombinant DNA construct with a ddRNAi construct of the disclosure, the DNA construct may comprise, in a 5′ to 3′ direction, the ddRNAi construct and the CAR construct. In accordance with another example in which the CAR construct is provided in a recombinant DNA construct with a ddRNAi construct of the disclosure, the DNA construct may comprise, in a 5′ to 3′ direction, the CAR construct and the ddRNAi construct.

As described herein, the present disclosure provides a CAR construct comprising a nucleic acid with a DNA sequence encoding a CAR, or a recombinant DNA construct comprising, wherein the CAR comprises an antigen binding domain. The antigen binding domain is, for example, a binding protein (e.g., antibody, or antibody fragment, TCR or TCR fragment), that binds specifically to a tumor antigen, e.g., a tumor antigen described herein, wherein the sequence of the antigen binding domain is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

In certain examples, a CAR construct of the disclosure comprises a sequence coding for a scFv, wherein the scFv may be preceded by an optional leader sequence, and followed by an optional hinge sequence, a transmembrane region, and/or an intracellular signaling domain, e.g., a costimulatory signaling domain. The domains may be contiguous and in the same reading frame to form a single fusion protein.

In one example, the CAR construct of the disclosure comprises a sequence coding for an optional leader sequence, an extracellular antigen binding domain e.g., a scFv, a hinge, a transmembrane domain, and an intracellular stimulatory domain.

In one example, the CAR construct of the disclosure comprises an optional leader sequence, an extracellular antigen binding domain, a hinge, a transmembrane domain, an intracellular costimulatory signaling domain (e.g., a costimulatory signaling domain) and/or an intracellular primary signaling domain.

In one example, a DNA construct of the disclosure comprises:
(a) a ddRNAi construct as described herein which comprises (i) a nucleic acid comprising or consisting of a DNA sequence set forth in SEQ ID NO:162 (shmiR-TCR-β_5) operably-linked to a U6 promoter e.g., a U6-9 promoter, and the transcription terminator sequence TTTTT (ii) a nucleic acid comprising or consisting of a DNA sequence set forth in SEQ ID NO:164 (shmiR-CD3-γ_2) operably-linked to a U6 promoter e.g., a U6-1 promoter, and the transcription terminator sequence TTTTT, (iii) a nucleic acid comprising or consisting of a DNA sequence set forth in SEQ ID NO:171 (shmiR-CD3-ε_3) operably-linked to a H1 promoter and the transcription terminator sequence TTTTT; and
(b) a CAR construct comprising nucleic acid with a DNA sequence coding for a CAR e.g., an anti-CD19 CAR.

For example, a DNA construct of the disclosure may comprise:
(a) a 5′ lentiviral terminal repeat (LTR) sequence;
(b) a ddRNAi construct comprising (i) a nucleic acid comprising or consisting of a DNA sequence set forth in SEQ ID NO:162 (shmiR-TCR-β_5) operably-linked to a U6 promoter e.g., a U6-9 promoter, and the transcription terminator sequence TTTTT (ii) a stuffer sequence e.g., an HPRT derived stuffer sequence, (iii) a nucleic acid comprising or consisting of a DNA sequence set forth in SEQ ID NO:164 (shmiR-CD3-γ_2) operably-linked to a U6 promoter e.g., a U6-1 promoter, and the transcription terminator sequence TTTTT, (iv)) a stuffer sequence e.g., an HPRT derived stuffer sequence, and (v) a nucleic acid comprising or consisting of a DNA sequence set forth in SEQ ID NO:171 (shmiR-CD3-ε_3) operably-linked to a H1 promoter and the transcription terminator sequence TTTTT; and
(c) a CAR construct comprising nucleic acid with a DNA sequence coding for a CAR e.g., an anti-CD19 CAR; and
(d) a 3′ LTR sequence.

One exemplary DNA construct of the disclosure may comprise or consist of a DNA sequence set forth in SEQ ID NO: 179.

Further CAR constructs, and components thereof, which may be included in a DNA construct of the disclosure are described herein.

### Antigen Binding Domains

A CAR as described herein will include an antigen binding domain in the extracellular region.

In one example, the antigen binding domain is a murine antibody or antibody fragment comprising an antigen binding domain. In one example, the antigen binding domain is a humanized antibody or antibody fragment comprising an antigen binding domain. In one example, the antigen binding domain is a human antibody or antibody fragment comprising an antigen binding domain.

The choice of an antigen binding domain can depend upon the type and number of ligands or receptors that define the surface of a target cell. For example, the antigen binding domain may be chosen to recognize an antigen that acts as a cell surface marker on target cells associated with a particular disease state. Examples of cell surface markers that may act as ligands or receptors include a cell surface marker associated with a particular disease state, e.g., cell surface makers for viral diseases, bacterial diseases parasitic infections, autoimmune diseases and disorders associated with unwanted cell proliferation, e.g., a cancer, e.g., a cancer described herein.

In certain examples, the antigen binding domain recognizes an antigen of a proliferative disorder e.g., cancer, including but not limited to primary or metastatic melanoma, thymoma, lymphoma, sarcoma, lung cancer (e.g., NSCLC or SCLC), liver cancer, non-Hodgkin's lymphoma, Hodgkin's lymphoma, leukemias, multiple myeloma, glioblastoma, neuroblastoma, uterine cancer, cervical cancer, renal cancer, thyroid cancer, bladder cancer, kidney cancer and adenocarcinomas such as breast cancer, prostate cancer, ovarian cancer, pancreatic cancer, colon cancer and the like. In some embodiments, the cancer is B-cell acute lymphoid leukemia (“BALL”), T-cell acute lymphoid leukemia (“TALL”), acute lymphoid leukemia (ALL), acute myelogenous leukemia (AML); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplasia syndrome, non-Hodgkin's lymphoma, plasmablastic lymphoma, plasmacytoid dendritic cell neoplasm, Waldenstrom macroglobulinemia.

In one example, the antigen binding domain binds specifically to a tumor antigen which comprises one or more antigenic cancer epitopes immunologically recognized by tumor infiltrating lymphocytes (TIL) derived from a cancer tumor of a mammal. Tumor antigens can be proteins that are produced by tumor cells that elicit an immune response, particularly T-cell mediated immune responses. The selection of the antigen binding domain of the dsiclosure will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, FAP, B7H3, Kit, CA-IX, CS-1, MUC1, BCMA, bcr-abl, HER2, β-human chorionic gonadotropin, alphafetoprotein (AFP), ALK, CD19, CD123, cyclin B1, lectin-reactive AFP, Fos-related antigen 1, ADRB3, thyroglobulin, EphA2, RAGE-1, RU1, RU2, SSX2, AKAP-4, LCK, OY-TES1, PAXS, SART3, CLL-1, fucosyl GM1, GloboH, MN-CA IX, EPCAM, EVT6-AML, TGSS, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYP1B1, BORIS, prostase, prostate-specific antigen (PSA), PAX3, PAP, NY-ESO-1, LAGE-1a, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A 1, MAD-CT-1, MAD-CT-2, Melan A/MART 1, XAGE1, ELF2M, ERG (TMPRSS2 ETS fusion gene), NA17, neutrophil elastase, sarcoma translocation breakpoints, NY-BR-1, ephrinB2, CD20, CD22, CD24, CD30, CD33, CD38, CD44v6, CD97, CD171, CD179a, androgen receptor, FAP, insulin growth factor (IGF)-I, IGF-II, IGF-I receptor, GD2, o-acetyl-GD2, GD3, GM3, GPRCSD, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In one example, the tumor antigen is selected from the group consisting of folate receptor (FRa), mesothelin, EGFRvIII, IL-13Ra, CD123, CD19, CD33, BCMA, GD2, CLL-1, CA-IX, MUC1, HER2, and any combination thereof. In one example, the tumor antigen is CD19.

In one example, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target antigens include transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD 19, CD20 and CD37 are other candidates for target antigens in B-cell lymphoma.

In some examples, the tumor antigen is a tumor antigen described in WO2015/120096, WO2015/142675, WO2016/019300, WO2016/011210, WO2016/109410 and WO2016/069283, the contents of which are incorporated by reference in their entirety.

Depending on the desired antigen to be targeted, the sequence encoding the CAR can be engineered to include the appropriate antigen binding domain that is specific to the desired antigen target.

A CAR construct as described herein may comprise a DNA sequence coding for an antigen binding domain (e.g., antibody or antibody fragment) that binds to a MHC presented-peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Bood, 2011, 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, a TCR-like antibody can be identified from screening a library, such as a human scFv phage displayed library. Accordingly, a CAR described herein may comprises an antigen binding domain that binds to a MHC presented peptide of a molecule selected from any tumor antigen described above that is expressed intracellularly, e.g., p53, BCR-Abl, Ras, K-ras, and c-met.

In one example, the CAR construct or recombinant DNA construct comprising same can include a further nucleic acid with a DNA sequence encoding a second CAR, e.g., a second CAR that includes a different antigen binding domain, e.g., to the same target (a cancer associated antigen as described herein) or a different target (e.g., CD19, CD123, CD22, CD30, CD34, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, TSHR, GPRCSD, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAXS, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRLS, or IGLL1). In accordance with an example where the DNA construct comprises nucleic acids encoding two or more different CARs, the antigen binding domains of the different CARs can be such that the antigen binding domains do not interact with one another. For example, the antigen binding domain of the first CAR, e.g., as a fragment (e.g., an scFv), will not form an association with the antigen binding domain of the second CAR. In one example, the antigen binding domain of the first or second CAR is a VHH.

The antigen binding domain of the CAR which is encoded by the CAR construct, or DNA construct comprising same, can be derived from an antibody molecule, e.g., one or more of monoclonal antibodies, polyclonal antibodies, recombinant antibodies, human antibodies, humanized antibodies, single-domain antibodies e.g., a heavy chain variable domain (VH), a light chain variable domain (VL) from e.g., human, and a variable domain (VHH). In some examples, it is beneficial for the antigen binding domain to be derived from the same species in which the CAR will ultimately be used in, e.g., for use in humans, it may be beneficial for the antigen binding domain of the CAR, described herein, to comprise a human or a humanized antigen binding domain.

In some examples, the antigen binding domain comprises a fragment of an antibody that is sufficient to confer recognition and specific binding to the target antigen. Examples of an antibody fragment include, but are not limited to, an Fab, Fab′, F(ab′)2, or Fv fragment, an scFv antibody fragment, a linear antibody, single domain antibody such as an sdAb (either VL or VH), a camelid VHH domain, and multi-specific antibodies formed from antibody fragments.

In one example, the antigen binding domain is a “scFv,” which can comprise a fusion protein comprising a VL chain and a VH chain of an antibody, where the VH and VL are, e.g., linked via a short flexible polypeptide linker, e.g., a linker described herein. The scFv is capable of being expressed as a single chain polypeptide and retains the specificity of the intact antibody from which it is derived. Moreover, the VL and VH variable chains can be linked in either order, e.g., with respect to the N-terminal and C-terminal ends of the polypeptide, the scFv may comprise VL-linker-VH or may comprise VH-linker-VL.

In some examples, the scFv molecules comprise flexible polypeptide linker with an optimized length and/or amino acid composition. The flexible polypeptide linker length can greatly affect how the variable regions of a scFv fold and interact. In fact, if a short polypeptide linker is employed (e.g., between 5-10 amino acids, intrachain folding is prevented. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

In some examples, the antigen binding domain is a single domain antigen binding (sdAb) molecule. A sdAb molecule includes molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies (e.g., described in more detail below). SDAB molecules may be any of the art, or any future single domain molecules. SDAB molecules may be derived from any species including, but not limited to mouse, human, camel, llama, fish, shark, goat, rabbit, and bovine.

In certain examples, the SDAB molecule is a single chain fusion polypeptide comprising one or more single domain molecules (e.g., nanobodies), devoid of a complementary variable domain or an immunoglobulin constant, e.g., Fc, region, that binds to one or more target antigens.

The SDAB molecules can be recombinant, CDR-grafted, humanized, camelized, de-immunized and/or in vitro generated (e.g., selected by phage display).

In one example, the antigen biding domain portion comprises a human antibody or a fragment thereof.

In some examples, a non-human antibody is humanized, where specific sequences or regions of the antibody are modified to increase similarity to an antibody naturally produced in a human. In an embodiment, the antigen binding domain is humanized.

In another example, the antigen binding domain of the CAR is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and vβ genes from a T cell clone linked by a linker (e.g., a flexible peptide). This approach is very useful to cancer associated target that itself is intracellular, however, a fragment of such antigen (peptide) is presented on the surface of the cancer cells by MHC.

In another example, a CAR construct of the disclosure comprise a DNA sequence coding for an antigen binding domain that binds specifically to a virus antigen or viral-induced antigen found on the surface of an infected cell. For example, the virus antigen or viral-induced antigen may be from a virus selected from the group consisting of Human cytomegalovirus (HCMV), Human immunodeficiency virus (HIV), Epstein-Barr virus (EBV), adenovirus (AdV), varicella zoster virus (VZV), influenza and BK virus (BKV), John Cunningham (JC) virus, respiratory syncytial virus (RSV), parainfluenzae, rhinovirus, human metapneumovirus, herpes simplex virus (HSV) 1, HSV II, human herpes virus (HHV) 6, HHV 8, Hepatitis A virus, Hepatitis B virus (HBV), Hepatitis C virus (HCV), hepatitis E virus, rotavirus, papillomavirus, parvovirus Ebola virus, zika virus, a hantavirus and vesicular stomatitis virus (VSV).

### Bispecific CARs

In some examples, the CAR construct, or recombinant DNA construct comprising same, comprises a DNA sequence encoding a bispecific CAR.

In one example, the bispecific CAR is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In one example, the first and second epitopes are on the same antigen, e.g., the same protein (or subunit of a multimeric protein). In one example, the first and second epitopes overlap. In one example, the first and second epitopes do not overlap. In one example, the first and second epitopes are on different antigens, e.g., different proteins (or different subunits of a multimeric protein). In one example, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In one example, a bispecific antibody molecule comprises a half antibody having binding specificity for a first epitope and a half antibody having binding specificity for a second epitope. In one example, a bispecific antibody molecule comprises a half antibody, or fragment thereof, having binding specificity for a first epitope and a half antibody, or fragment thereof, having binding specificity for a second epitope. In one example, a bispecific antibody molecule comprises a scFv, or fragment thereof, have binding specificity for a first epitope and a scFv, or fragment thereof, have binding specificity for a second epitope.

In one example, the bispecific CAR is a multi-specific (e.g., a bispecific or a trispecific) antibody molecule.

Within each antibody or antibody fragment (e.g., scFv) of a bispecific antibody molecule, the VH can be upstream or downstream of the VL. In one example, the upstream antibody or antibody fragment (e.g., scFv) is arranged with its VH (VH1) upstream of its VL (VL1) and the downstream antibody or antibody fragment (e.g., scFv) is arranged with its VL (VL2) upstream of its VH (VH2), such that the overall bispecific antibody molecule has the arrangement VH1-VL1-VL2-VH2. In another example, the upstream antibody or antibody fragment (e.g., scFv) is arranged with its VL (VL1) upstream of its VH (VH1) and the downstream antibody or antibody fragment (e.g., scFv) is arranged with its VH (VH2) upstream of its VL (VL2), such that the overall bispecific antibody molecule has the arrangement VL1-VH1-VH2-VL2. Optionally, a linker is disposed between the two antibodies or antibody fragments (e.g., scFvs), e.g., between VL1 and VL2 if the construct is arranged as VH1-VL1-VL2-VH2, or between VH1 and VH2 if the construct is arranged as VL1-VH1-VH2-VL2. In general, the linker between the two scFvs should be long enough to avoid mispairing between the domains of the two scFvs.

Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

In one example, the bispecific antibody molecule is characterized by a first immunoglobulin variable domain sequence, e.g., a scFv, which has binding specificity for a first cancer-associated antigen, e.g., comprises a scFv as described herein, or comprises the light chain CDRs and/or heavy chain CDRs from a scFv described herein, and a second immunoglobulin variable domain sequence that has binding specificity for a second epitope on a different antigen.

### Chimeric TCR

In some examples, the CAR construct, or recombinant DNA construct comprising same, comprises a DNA sequence encoding a chimeric TCR. For example, the antigen binding domain of the CAR can be linked to one or more constant domain of a T cell receptor (“TCR”) chain, for example, a TCR alpha or TCR beta chain, to create an chimeric TCR that binds specifically to a cancer associated antigen. Without being bound by theory, it is believed that chimeric TCRs will signal through the TCR complex upon antigen binding. For example, a scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR that binds specifically to a cancer associated antigen. For example, the LC CDRs disclosed herein may be grafted into the variable domain of a TCR alpha chain and the HC CDRs disclosed herein may be grafted to the variable domain of a TCR beta chain, or vice versa.

### Non-Antibody Scaffolds

In some examples, the CAR construct, or recombinant DNA construct comprising same, comprises a DNA sequence encoding CAR comprising an antigen binding domain having a non-antibody scaffold, e.g., a fibronectin, ankyrin, domain antibody, lipocalin, small modular immuno-pharmaceutical, maxybody, Protein A, or affilin. The non-antibody scaffold has the ability to bind to target antigen on a cell. In one example, the antigen binding domain is a polypeptide or fragment thereof of a naturally occurring protein expressed on a cell. In one example, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

Fibronectin scaffolds can be based on fibronectin type III domain (e.g., the tenth module of the fibronectin type III (10 Fn3 domain)). The fibronectin type III domain has 7 or 8 beta strands which are distributed between two beta sheets, which themselves pack against each other to form the core of the protein, and further containing loops (analogous to CDRs) which connect the beta strands to each other and are solvent exposed. There are at least three such loops at each edge of the beta sheet sandwich, where the edge is the boundary of the protein perpendicular to the direction of the beta strands (see U.S. Pat. No. 6,818,418). Because of this structure, this non-antibody scaffold mimics antigen binding properties that are similar in nature and affinity to those of antibodies.

The ankyrin technology is based on using proteins with ankyrin derived repeat modules as scaffolds for bearing variable regions which can be used for binding to different targets. The ankyrin repeat module is a 33 amino acid polypeptide consisting of two anti-parallel α-helices and a β-turn. Binding of the variable regions is mostly optimized by using ribosome display.

Avimers are derived from natural A-domain containing protein such as HER3. These domains are used by nature for protein-protein interactions and in human over 250 proteins are structurally based on A-domains. Avimers consist of a number of different “A-domain” monomers (2-10) linked via amino acid linkers. Avimers can be created that can bind to the target antigen using the methodology described in, for example, U.S. Patent Application Publication Nos. 20040175756; 20050053973; 20050048512; and 20060008844.

Affibody affinity ligands are small, simple proteins composed of a three-helix bundle based on the scaffold of one of the IgG-binding domains of Protein A. Protein A is a surface protein from the bacterium Staphylococcus aureus. This scaffold domain consists of 58 amino acids, 13 of which are randomized to generate affibody libraries with a large number of ligand variants (See e.g., U.S. Pat. No. 5,831,012). Affibody molecules mimic antibodies, they have a molecular weight of 6 kDa, compared to the molecular weight of antibodies, which is 150 kDa. In spite of its small size, the binding site of affibody molecules is similar to that of an antibody.

Protein epitope mimetics (PEM) are medium-sized, cyclic, peptide-like molecules (MW 1-2 kDa) mimicking beta-hairpin secondary structures of proteins, the major secondary structure involved in protein-protein interactions. Antigen binding domains, e.g., those comprising scFv, single domain antibodies, or camelid antibodies, can be directed to any target receptor/ligand described herein.

In one example, the antigen binding domain comprises the extracellular domain, or a counter-ligand binding fragment thereof, of molecule that binds a counterligand on the surface of a target cell.

An antigen binding domain can comprise the extracellular domain of an inhibitory receptors. Engagement with a counterligand of the coinhibitory molecule is redirected into an optimization of immune effector response.

An antigen binding domain can comprise the extracellular domain of a costimulatory molecule, referred to as a Costimulatory ECD domain. Engagement with a counter ligand of the costimulatory molecule results in optimization of immune effector response.

### Transmembrane Domain

In some examples, the CAR construct, or recombinant DNA construct comprising same, comprises a DNA sequence encoding CAR which comprises a transmembrane domain that is fused to an extracellular sequence, e.g., an extracellular recognition element, which can comprise an antigen binding domain, an inhibitory counter ligand binding domain, or a costimulatory ECD domain. In one example, the transmembrane domain is one that naturally is associated with one of the domains in the CAR. In one example, the transmembrane domain is one that is not naturally associated with one of the domains in the CAR.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one example, the transmembrane domain is one that is associated with one of the other domains of the CAR e.g., the transmembrane domain may be from the same protein that the signaling domain, co-stimulatory domain or the hinge domain is derived from. In another example, the transmembrane domain is not derived from the same protein that any other domain of the CAR is derived from.

In one example, the transmembrane domain is one which minimizes interactions with other elements, e.g., other transmembrane domains. In some instances, the transmembrane domain minimizes binding of such domains to the transmembrane domains of the same or different surface membrane proteins, e.g., to minimize interactions with other members of the receptor complex. Suitable examples can be derived by selection or modification of amino acid substitution of a known transmembrane domain. In one example, the transmembrane domain is capable of promoting homodimerization with another CAR on the cell surface. In another example, the amino acid sequence of the transmembrane domain may be modified or substituted so as to minimize interactions with the binding domains of the native binding partner present in the same CAR-expressing cell.

The transmembrane domain may comprise a naturally occurring, or a non-naturally occurring synthetic sequence. Where naturally occurring, the transmembrane domain may be derived from any membrane-bound or transmembrane protein.

A CAR encoded by the recombinant DNA construct of the disclosure may comprises a transmembrane region derived from any one or more of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD 162), LTBR, PAG/Cbp, NKG2D, and NKG2C.

In one example, a sequence, e.g., a hinge or spacer sequence, can be disposed between a transmembrane domain and another sequence or domain to which it is fused. In some examples, a variety of human hinges (aka “spacers”) can be employed as well, e.g., including but not limited to the human Ig (immunoglobulin) hinge. In one example, the hinge can be a human Ig (immunoglobulin) hinge (e.g., an IgG4 hinge an IgD hinge), a GS linker (e.g., a GS linker described herein), a KIR2DS2 hinge or a CD8a hinge. Optionally, a short oligo- or polypeptide linker, between 2 and 10 amino acids in length may form the linkage between the transmembrane domain and another domain, e.g., an intracellular signaling domain or costimulatory domain, of a CAR. A glycine-serine doublet provides a particularly suitable linker.

In one example, the transmembrane domain may be a non-naturally occurring sequence, in which case can comprise predominantly hydrophobic residues such as leucine and valine. In an embodiment, a triplet of phenylalanine, tryptophan and valine will be found at each end of a transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker.

### Intracellular Signaling Domain

In some examples, the CAR construct, or recombinant DNA construct comprising same, comprises a DNA sequence encoding a CAR comprising an intracellular signalling domain. An intracellular signaling domain produces an intracellular signal when an extracellular domain, e.g., an antigen binding domain, to which it is fused, binds a counter ligand. Intracellular signaling domains can include primary intracellular signaling domains and costimulatory signaling domains. In one example, a CAR molecule can be constructed for expression in an immune cell, e.g., a T cell, such that the CAR molecule comprises a domain, e.g., a primary intracellular signaling domains, costimulatory signaling domain, inhibitory domains, etc., that is derived from a polypeptide that is typically associated with the immune cell. By way of example only, a CAR for expression in a T cell can comprise a 41BB domain and a CD3 zeta domain. In accordance with this example, both the 41BB and CD3 zeta domains are derived from polypeptides associated with the T cell. In yet another example, a CAR for expression in a T cell can comprise a CD28 domain and a CD3 zeta domain. In another example, a CAR for expression in a T cell can comprise an ICOS domain and a CD3 zeta domain. In another example, a CAR for expression in a T cell can comprise a CD27 domain and a CD3 zeta domain. In another example, a CAR molecule can be constructed for expression in an immune cell e.g., a T cell, such that the CAR molecule comprises a domain that is derived from a polypeptide that is not typically associated with the immune cell.

### Primary Intracellular Signaling Domain

In some examples, the CAR construct, or recombinant DNA construct comprising same, comprises a DNA sequence encoding a CAR comprising a primary intracellular signalling domain. A primary intracellular signaling domain produces an intracellular signal when an extracellular domain, e.g., an antigen binding domain, to which it is fused binds cognate antigen. The primary intracellular signaling domain is derived from a primary stimulatory molecule, e.g., it comprises intracellular sequence of a primary stimulatory molecule. The primary intracellular signaling domain comprises sufficient primary stimulatory molecule sequence to produce an intracellular signal, e.g., when an antigen binding domain to which it is fused binds cognate antigen.

A primary stimulatory molecule, is a molecule, that upon binding cognate ligand, mediates an immune effector response, e.g., in the cell in which it is expressed. Typically, it generates an intracellular signal that is dependent on binding to a cognate ligand that comprises antigen. The TCR/CD3 complex is an exemplary primary stimulatory molecule; it generates an intracellular signal upon binding to cognate ligand, e.g., an MHC molecule loaded with a peptide. Typically, e.g., in the case of the TCR/CD3 primary stimulatory molecule, the generation of an intracellular signal by a primary intracellular signaling domain is dependent on binding of the primary stimulatory molecule to antigen. Primary stimulation can mediate altered expression of certain molecules, such as downregulation of TGF-β, and/or reorganization of cytoskeletal structures, and the like.

Stimulation, can, e.g., in the presence of costimulation, result in an optimization, e.g., an increase, in an immune effector function of the CART cell. Stimulation, e.g., in the context of a CART cell, can mediate a T cell response, e.g., proliferation, activation, differentiation, and the like.

In one example, the primary intracellular signaling domain comprises a signaling motif, e.g., an immunoreceptor tyrosine-based activation motif or ITAMs. A primary intracellular signaling domain can comprise ITAM containing cytoplasmic signaling sequences from (for example) TCR zeta (CD3 zeta), common FcR gamma, (FCER1G), Fc gamma R11a, FcR beta (Fc Epsilon Rib), CD3 gamma, CD3 delta, CD3 epsilon, CD5, CD22, CD79a, CD79b, CD278 (also known as “ICOS”), FcsRI, DAP10, DAP 12, and CD66d.

A primary intracellular signaling domain comprises a functional fragment, or analog, of a primary stimulatory molecule (e.g., CD3 zeta). The primary intracellular signaling domain can comprise the entire intracellular region or a fragment of the intracellular region which is sufficient for generation of an intracellular signal when an antigen binding domain to which it is fused binds cognate antigen. In some examples, the primary intracellular signaling domain has at least 70, 75, 80, 85, 90, 95, 98, or 99% sequence identity with the entire intracellular region, or a fragment of the intracellular region which is sufficient for generation of an intracellular signal, of a naturally occurring primary stimulatory molecule, e.g., a human, or other mammalian, e.g., a nonhuman species, e.g., rodent, monkey, ape or murine intracellular primary stimulatory molecule.

In some examples, the primary intracellular signaling domain has at least 70, 75, 80, 85, 90, 95, 96, 97, 98, or 99% identity with, or differs by no more than 30, 25, 20, 15, 10, 5, 4, 3, 2, or 1 amino acid residues from the corresponding residues of the entire intracellular region, or a fragment of the intracellular region which is sufficient for generation of an intracellular signal, of a naturally occurring human primary stimulatory molecule, e.g., a naturally occurring human primary stimulatory molecule disclosed herein.

### Costimulatory Signaling Domain

In some examples, the CAR construct, or recombinant DNA construct comprising same, comprises a DNA sequence encoding a CAR comprising a costimulatory signaling domain which produces an intracellular signal when an extracellular domain, e.g., an antigen binding domain, to which it is fused binds cognate ligand. The costimulatory signaling domain is derived from a costimulatory molecule. The costimulatory signaling domain comprises sufficient primary costimulatory molecule sequence to produce an intracellular signal, e.g., when an extracellular domain, e.g., an antigen binding domain, to which it is fused binds cognate ligand.

The costimulatory domain can be one which optimizes the performance, e.g., the persistence, or immune effector function, of a T cell that comprises a CAR which comprises the costimulatory domain.

Costimulatory molecules are cell surface molecules, other than antigen receptors or their counter ligands that promote an immune effector response. In some cases they are required for an efficient or enhanced immune response. Typically, a costimulatory molecule generates an intracellular signal that is dependent on binding to a cognate ligand that is, in certain examples, other than an antigen, e.g., the antigen recognized by an antigen binding domain of a CART cell. Typically, signaling from a primary stimulatory molecule and a costimulatory molecule contribute to an immune effector response, and in some cases both are required for efficient or enhanced generation of an immune effector response.

A costimulatory domain comprises a functional fragment, or analog, of a costimulatory molecule (e.g., ICOS, CD28, or 4-1BB). It can comprise the entire intracellular region or a fragment of the intracellular region which is sufficient for generation of an intracellular signal, e.g., when an antigen binding domain to which it is fused binds cognate antigen. In certain examples, the costimulatory domain has at least 70%, 75%, 80%, 85%, 90%, 95%, 98%, or 99% sequence identity with the entire intracellular region, or a fragment of the intracellular region which is sufficient for generation of an intracellular signal, of a naturally occurring costimulatory molecule, e.g., a human, or other mammalian, e.g., a nonhuman species, e.g., rodent, monkey, ape or murine intracellular costimulatory molecule.

Exemplary co-stimulatory domains include, by are no limited to, those selected from CD27, CD27, CD28, 4-1BB (CD137), QX40, CD30, CD40, ICQS (CD278), ICAM-1, LFA-1 (CD11a/CD18), CD2, CD7, LIGHT, NKG2C, B7-H3, a ligand that specifically binds with CD8, CD5, GITR, BAFFR, HVEM (LIGHTR), SLAMf7, NKP80 (KLRF1), CD160 (BY55), CD19, CD4, CD8 alpha, CD8 beta, IL2R beta, IL2R gamma, IL7R alpha, ITGA4, VLA1, CD49a, ITGA4, IA4, CD49D, ITGA6, VLA-6, C49f, ITGAD, CDlld, ITGAE, CD103, ITGAL, ITGAM, CDllb, ITGAX, CDllc, ITGB1, CD29, ITGB2, CD18, ITGB7, TNFR2, TRANCE/RANKL, DNAM1 (CD226), SLAMF4 (C244, 2B4), CD84, CD96 (Tactile), CEACAM1, CRTAM, Ly9 (CD229), PSGL1, C100 (SEMA4D), CD69, SLAMF6 (NTB-A, Ly108), SLAM (SLAMF1, CD150, IP0-3), BLAME (SLAMF8), SELPLG (CD162), LTBR, LAT, GADS, and PAG/Cbp.

In some examples, the costimulatory signaling domain has at least 70, 75, 80, 85, 90, 95, 96, 97, 98, or 99% identity with, or differs by no more than 30, 25, 20, 15, 10, 5, 4, 3, 2, or 1 amino acid residues from the corresponding residues of the entire intracellular region, or a fragment of the intracellular region which is sufficient for generation of an intracellular signal, of, a naturally occurring human costimulatory molecule, e.g., a naturally occurring human costimulatory molecule disclosed herein.

### Costimulatory Molecule Ligand Binding Domains

In some examples, the CAR construct, or recombinant DNA construct comprising same, comprises a DNA sequence encoding a CAR comprising an extracellular ligand binding domain of a costimulatory molecule, referred to as a costimulatory ECD domain, coupled to a intracellular signaling domain that promotes an immune effector response. Thus, engagement with a counter ligand of the costimulatory molecule results in optimization of immune effector response.

Exemplary Costimulatory ECD domains from costimulatory molecules (identified by the Costimulatory Molecules from which they are derived) include, but are not limited to, ICOS, CD28, CD27, HVEM, LIGHT, CD40L, 4-1BB, OX40, DR3, GITR, CD30, TIM1, SLAM, CD2 and CD226.

In some examples, the Costimulatory ECD domain has at least 70, 75, 80, 85, 90, 95, 96, 97, 98, or 99% identity with, or differs by no more than 30, 25, 20, 15, 10, 5, 4, 3, 2, or 1 amino acid residues from the corresponding residues of the entire extracellular region, or a fragment of the extracellular region which is sufficient for engagement with the counter ligand, of a naturally occurring human inhibitory molecule, e.g., a naturally occurring human costimulatory molecule disclosed herein.

### Inhibitory CAR Members

In some examples, the CAR construct, or recombinant DNA construct comprising same, comprises a DNA sequence encoding a CAR comprising an inhibitory CAR (iCAR) member. An iCAR member comprises: an antigen binding domain (or other extracelluar domain) that recognizes an antigen on a non-target, e.g., a noncancer, cell; a transmembrane domain; and, a domain from an inhibitory molecule, e.g., an intracellular domain from an inhibitory molecule. In one example, the iCAR member can comprise a second inhibitory intracellular signaling domain.

Upon engagement of the antigen binding domain (or other extracelluar domain) of the iCAR member with its target antigen (or counter-ligand), the iCAR contributes to inhibiting, e.g., reversibly inhibiting, or minimizing, activation of the cell comprising the iCAR. As such, inclusion of an iCAR member in a CAR, e.g., and CAR-T cell expressing the CAR, can limit damage to non-target, e.g., bystander, cells. While not wishing to be bound by theory, it is believed that an iCAR member, upon engagement with its antigen (or counter-ligand), limits one or more of cytokine secretion, cytotoxicity, and proliferation. In certain examples, the effect is temporary, and upon subsequent engagement with a target cell the CAR, e.g., CAR-T cell, is activated and attacks the target cell.

A target antigen for an iCAR member can be an antigen that has an expression profile on target cells and non-target cells such that an acceptably high level of attack on target cells and an acceptably low level of attack on non-target cells is achieved. Not only choice of antigen, but iCAR affinity for its antigen (or counter-ligand), CAR affinity for its antigen, level of expression of the iCAR, or levels of expression of the CAR can be used to optimize the ratio of on-target/off-target response.

In one example, the antigen is absent, or down-regulated on tumor cells. In one example, the antigen comprises an HLA molecule. In one example, the antigen comprises a cell surface tumor suppressor antigen. In one example, the antigen comprises PCML (or another antigen that is down-regulated in lymphomas, breast or prostate cancer), HYAL2, DCC, or SMAR1.

In one example, the antigen comprises a protein, carbohydrate, lipid, or a post-translational modification of a cell surface moiety, e.g., a mucin-type O-glycan (a core 3 O-glycan).

In one example, the antigen comprises a moiety that is down-regulated by tumor cells undergoing an epithelial to mesenchymal transition.

In one example, the antigen comprises E-cadherin.

In one example, a domain from an inhibitory molecule produces an intracellular signal when an extracellular domain, e.g., an antigen binding domain, to which it is fused binds cognate antigen (or counter ligand). The inhibitory intracellular signaling domain is derived from an inhibitory molecule, e.g., it comprises intracellular sequence of an inhibitory molecule. It comprises sufficient inhibitory molecule sequence to produce an intracellular signal, e.g., when an antigen binding domain to which it is fused binds its cognate antigen.

In one example, the primary intracellular signaling domain comprises a signaling motif, e.g., an immunoreceptor tyrosine-based activation motif or TTIM.

A domain from an inhibitory molecule comprises a functional fragment, or analog, of an inhibitory molecule intracellular domain. It can comprise the entire intracellular region or a fragment of the intracellular region which is sufficient for generation of an intracellular signal when an antigen binding domain to which it is fused, binds cognate antigen. In one example, the inhibitory intracellular signaling domain has at least 70, 75, 80, 85, 90, 95, 98, or 99% sequence identity with, or differs by no more than 30, 25, 20, 15, 10, 5, 4, 3, 2, or 1 amino acid residues from, the corresponding residues of a naturally occurring inhibitory molecule, e.g., such as a molecule selected from B7-H1, B7-1, CD160, P1H, 2B4, PD1, TIM3, LAG3, TIGIT, CTLA-4, BTLA, LAIR1 and TGF-beta receptor.

Thus, in one example, the recombinant DNA construct of the disclosure comprises a CAR comprising an iCAR member. The iCAR member may comprise: an antigen binding domain (or other extracelluar domain) that recognizes an antigen on a non-target, e.g., a noncancer cell; a transmembrane domain; and a domain from an inhibitory molecule—e.g., as described herein.

### Expression Vectors

In one example, the ddRNAi construct or the CAR construct of the disclosure, or the DNA construct comprising the ddRNAi construct and the CAR construct of the disclosure, is/are included within an expression vector or expression vector(s).

In one example, the ddRNAi construct and the CAR construct are separately included in a single expression vector. In another example, the DNA construct is included in a single expression vector. In another example, the ddRNAi construct and the CAR construct are included in separate expression vectors. In accordance with an example in which the ddRNAi construct and the CAR construct are included in separate expression vectors, the respective expression vectors may be the same or different.

In one example, the or each expression vector is a plasmid e.g., as is known in the art. In one example, a suitable plasmid expression vector is a pBL vector. As described herein, the plasmid may comprise one or more promoters (suitable examples of which are described) to drive expression of the shmiRs of the disclosure.

In one example, the or each expression vector is mini-circle DNA. Mini-circle DNA is described in U.S. Patent Publication No. 2004/0214329. Mini-circle DNA are useful for persistently high levels of nucleic acid transcription. The circular vectors are characterized by being devoid of expression-silencing bacterial sequences. For example, mini-circle vectors differ from bacterial plasmid vectors in that they lack an origin of replication, and lack drug selection markers commonly found in bacterial plasmids, e.g., β-lactamase, tet, and the like. Consequently, minicircle DNA becomes smaller in size, allowing more efficient delivery.

In one example, the or each expression vector is a viral vector.

A viral vector based on any appropriate virus may be used to deliver a ddRNAi and/or CAR construct of the disclosure. In addition, hybrid viral systems may be of use. The choice of viral delivery system will depend on various parameters, such as the tissue targeted for delivery, transduction efficiency of the system, pathogenicity, immunological and toxicity concerns, and the like.

Commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses). In one example, a viral vector of the disclosure integrates into a host cell's chromatin. In another example, a viral vector of the disclosure persists in a host cell's nucleus as an extrachomosomal episome.

In some examples, a viral vector of the disclosure is a lentivirus. Lentivirus vectors are often pseudotyped with vesicular steatites virus glycoprotein (VSV-G), and have been derived from the human immunodeficiency virus (HIV); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization. One of the main advantages to the use of lentiviral vectors is that gene transfer is persistent in most tissues or cell types, even following cell division of the transduced cell.

A lentiviral-based construct used to express shmiRs from a ddRNAi construct of the disclosure and/or used to express a CAR from the CAR construct of the disclosure (including when provided in a DNA construct with a ddRNAi construct of the disclosure), comprises sequences from the 5′ and 3′ long terminal repeats (LTRs) of a lentivirus. In one example, the viral construct comprises an inactivated or self-inactivating 3′ LTR from a lentivirus. The 3′ LTR may be made self-inactivating by any method known in the art. For example, the U3 element of the 3′ LTR contains a deletion of its enhancer sequence, e.g., the TATA box, Sp1 and NF-kappa B sites. As a result of the self-inactivating 3′ LTR, the provirus that is integrated into the host genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

In one example, a viral vector is an adenoviral (AdV) vector. Adenoviruses are medium-sized double-stranded, non-enveloped DNA viruses with linear genomes that is between 26-48 Kbp. Adenoviruses gain entry to a target cell by receptor-mediated binding and internalization, penetrating the nucleus in both non-dividing and dividing cells. Adenoviruses are heavily reliant on the host cell for survival and replication and are able to replicate in the nucleus of vertebrate cells using the host's replication machinery.

In one example, a viral vector is from the Parvoviridae family. The Parvoviridae is a family of small single-stranded, non-enveloped DNA viruses with genomes approximately 5000 nucleotides long. Included among the family members is adeno-associated virus (AAV). In one example, a viral vector of the disclosure is an AAV. AAV is a dependent parvovirus that generally requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells. Because progeny virus is not produced from AAV infection in the absence of helper virus, the extent of transduction is restricted only to the initial cells that are infected with the virus. It is this feature which makes AAV a desirable vector for the present disclosure. Furthermore, unlike retrovirus, adenovirus, and herpes simplex virus, AAV appears to lack human pathogenicity and toxicity (Kay, et al., Nature. 424: 251 (2003)). Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as shmiRs and shRNAs.

Another viral delivery system useful with a ddRNAi construct, CAR construct and/or DNA construct of the disclosure, is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

Other viral or non-viral systems known to those skilled in the art may be used to deliver the ddRNAi or nucleic acid of the present disclosure to cells of interest, including but not limited to gene-deleted adenovirus-transposon vectors (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus.

### Testing a Construct or Vector

ddRNAi Constructs Activity of a ddRNAi construct of the disclosure to inhibit expression of TCR complex subunits may be determined by introducing a ddRNAi construct, or expression vector comprising same, to a T-cell and subsequently measuring the level of expression of a RNA or protein encoded by the TCR complex subunit being targeted by the shmiRs in the ddRNAi construct. Levels of expression can be assayed either by a Taqman™ assay or other real time PCR assay designed for the specific TCR subunit or by ELISA for a TCR e.g., using commercially available antibodies and/or ELISA kits.

An exemplary method for determining downregulation of TCR subunit expression by individual shmiRs encoded by a ddRNAi construct of the disclosure are described in Example 3.

An exemplary method for determining downregulation of TCR subunit surface expression (i.e., expression and assembly of TCR on a cell surface) by individual shmiRs encoded by a ddRNAi construct of the disclosure are described in Example 5.

CAR Constructs and DNA Constructs Comprising Same Activity of a CAR construct or DNA construct of the disclosure to express a CAR may be determined by introducing a CAR construct, DNA construct, or expression vector comprising same, to a T-cell e.g., a T-cell comprising a non-functional endogenous TCR, and subsequently measuring the level of expression of an RNA or protein encoded by the CAR. Levels of expression can be assayed either by a Taqman™ assay or other real time PCR assay or by ELISA for the CAR.

### Compositions and Carriers

In some examples, the ddRNAi construct, CAR construct, DNA construct and/or expression vector(s) of the disclosure is/are provided in a composition or multiple compositions. For example, the composition is formulated such that it is can be introduced to a T-cell or a population of T-cells.

For example, a composition of the disclosure may comprise (i) an expression vector comprising a ddRNAi construct of the disclosure, (ii) an expression vector comprising a ddRNAi construct of the disclosure and an expression vector comprising a CAR construct of the disclosure, or (iii) an expression vector comprising a DNA construct of the disclosure. According to an example in which the ddRNAi construct and CAR construct are provided in different expression vectors, each expression vector may be provided in as separate composition e.g., which are packaged together.

A composition of the disclosure may also comprise one or more carriers or diluents e.g., suitable for use with T-cells. In one example, the carrier(s) or diluent(s) may be pharmaceutically acceptable. In one example, the carrier may be formulated to assist with introduction of the the ddRNAi construct, CAR construct, DNA construct and/or expression vector(s) of the disclosure to a T-cell e.g., in cell culture.

In some examples, the carrier is a lipid-based carrier, cationic lipid, or liposome nucleic acid complex, a liposome, a micelle, a virosome, a lipid nanoparticle or a mixture thereof.

In some examples, the carrier is a biodegradable polymer-based carrier, such that a cationic polymer-nucleic acid complex is formed. Use of cationic polymers for delivery compositions to cells is known in the art, such as described in Judge et al. Nature 25: 457-462 (2005), the contents of which is incorporated herein by reference.

In a further example, the carrier is a cyclodextrin-based carrier such as a cyclodextrin polymer-nucleic acid complex.

In a further example, the carrier is a protein-based carrier such as a cationic peptide-nucleic acid complex.

In another example, the carrier is a lipid nanoparticle. Exemplary nanoparticles are described, for example, in U.S. Pat. No. 7,514,099.

In some examples, the ddRNAi construct, CAR construct, DNA construct and/or expression vector(s) of the disclosure may be formulated with a lipid nanoparticle composition comprising a cationic lipid/Cholesterol/PEG-C-DMA/DSPC (e.g., in a 40/48/2/10 ratio), a cationic lipid/Cholesterol/PEG-DMG/DSPC (e.g., in a 40/48/2/10 ratio), or a cationic lipid/Cholesterol/PEG-DMG (e.g., in a 60/38/2 ratio). In some examples, the cationic lipid is Octyl CL in DMA, DL in DMA, L-278, DLinKC2DMA, or MC3.

In another example, the ddRNAi construct, CAR construct, DNA construct and expression vector(s) of the disclosure may be formulated with any of the cationic lipid formulations described in WO 2010/021865; WO 2010/080724; WO 2010/042877; WO 2010/105209 or WO 2011/022460.

In another example, the ddRNAi construct, CAR construct, DNA construct and expression vector(s) of the disclosure may be conjugated to or complexed with another compound, e.g., to facilitate delivery. Non-limiting, examples of such conjugates are described in US 2008/0152661 and US 2004/0162260 (e.g., CDM-LBA, CDM-Pip-LBA, CDM-PEG, CDM-NAG, etc.).

In another example, polyethylene glycol (PEG) is covalently attached to a ddRNAi construct, CAR construct, DNA construct and expression vector(s) of the disclosure. The attached PEG can be any molecular weight, e.g., from about 100 to about 50,000 daltons (Da).

In yet other example, the ddRNAi construct, CAR construct, DNA construct and expression vector(s) of the disclosure may be formulated with a carrier comprising surface-modified liposomes containing poly(ethylene glycol) lipids (PEG-modified, or long-circulating liposomes or stealth liposomes), such as is disclosed in for example, WO 96/10391; WO 96/10390; or WO 96/10392.

Other carriers include cyclodextrins (see for example, Gonzalez et al., 1999, Bioconjugate Chem., 10, 1068-1074; or WO 03/46185), poly(lactic-co-glycolic)acid (PLGA) and PLCA microspheres (see for example US 2002130430).

Compositions will desirably include materials that increase the biological stability of the ddRNAi construct, CAR construct, DNA construct and expression vector(s) of the disclosure and/or materials that increase the ability of the compositions to localise to T-cells. The therapeutic compositions of the disclosure may be administered in pharmaceutically acceptable carriers (e.g., physiological saline).

### T-Cells and Formulations Comprising Same

In one example, the present disclosure provides T-cell comprising a ddRNAi construct described herein, or a DNA construct described herein, or an expression vector described herein. A T-cell in accordance with this example does not express a functional TCR i.e., does not express an endogenous TCR. In one example, the T-cell exhibits reduced cell-surface expression of at least two components of the TCR complex. In one example, the T-cell exhibits reduced cell-surface expression of at least three components of the TCR complex. In one example, the T cell comprises a CAR construct as described herein and expresses a chimeric antigen receptor (CAR). Accordingly, a T-cell may be a CAR-T cell.

The CAR-T cell may express an antigen binding domain e.g., as described herein. In one example, the antigen binding domain is an antibody or an antigen binding domain thereof e.g., as herein before described. In one example, the antigen binding domain binds specifically to a tumor antigen e.g., as hereinbefore described. In another example, the antigen binding domain binds specifically to a viral antigen expressed on the surface of a cell e.g., a viral antigen as hereinbefore described.

In one example, the T-cell may be present in a subpopulation of T-cells which have been selected for particular properties e.g., based on HLA typing and/resistance to an immunosuppressant.

T-cells of the disclosure may be formulated for administration in adoptive T-cell therapy.

Formulation of the composition to be administered will vary according to the route of administration and formulation (e.g., solution, emulsion) selected. An appropriate pharmaceutical composition comprising the composition of the present disclosure to be administered can be prepared in a physiologically acceptable carrier. A mixture of compositions can also be used. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate.

The optimum concentration of cell populations in the chosen medium can be determined empirically, according to procedures well known to the skilled artisan, and will depend on the ultimate pharmaceutical formulation desired.

### Methods of Producing T-Cells

The present disclosure also provides methods of producing T-cells of the disclosure.

In one example, a method of producing a T-cell which does not express a functional TCR is provided, wherein the method comprises introducing into a T-cell a ddRNAi construct of the disclosure or an expression vector or a composition comprising same as described herein.

In another example, a method of inhibiting expression of two or more TCR complex subunits in a T-cell is provided, wherein the method comprises introducing into a T-cell a ddRNAi construct of the disclosure or an expression vector or a composition comprising same as described herein.

In another example, a method of producing a T-cell which does not express a functional TCR and which expresses a CAR is provided, wherein the method comprises introducing into a T-cell a DNA construct of the disclosure or an expression vector or composition comprising same as described herein.

The ddRNAi construct, CAR construct, DNA construct, and/or expression vector of the disclosure may be introduced to the T-cells using any suitable method known in the art. In some examples, the ddRNAi construct, CAR construct, DNA construct, and/or expression vector of the disclosure is introduced into the T-cells using recombinant infectious virus particles, such as e.g., vectors derived from simian virus 40 (SV40), adenoviruses, adeno-associated virus (AAV).

In some examples, the ddRNAi construct, CAR construct, DNA construct, and/or expression vector of the disclosure is introduced into the T-cells using recombinant lentiviral vectors or retroviral vectors, such as gamma-retroviral vectors e.g., as described herein. Methods of lentiviral transduction are known in the art and contemplated herein. Exemplary methods are described in e.g., Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

In some examples, the ddRNAi construct, CAR construct, DNA construct, and/or expression vector of the disclosure is introduced into the T cells via electroporation {see, e.g., Chicaybam et al, (2013) PLoS ONE 8(3): e60298 and Van Tedeloo et al. (2000) Gene Therapy 7(16): 1431-1437). In other examples, the ddRNAi construct, CAR construct, DNA construct, and/or expression vector of the disclosure is introduced into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells e.g., T-cells, include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some examples, prior to introducing the ddRNAi construct, CAR construct, DNA construct, and/or expression vector of the disclosure to the T-cell, T-cells can be obtained e.g., from a subject or a cell bank. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. Alternatively, T cell lines commercially available in the art, may be used.

In some examples, T cells can be obtained from a unit of blood collected from a subject using any number of techniques known to the skilled artisan, such as Ficoll™ separation. In another example, cells from the circulating blood of an individual are obtained by apheresis. T-cells collected by apheresis may be washed to remove the plasma fraction and optionally placed in an appropriate buffer or media for subsequent processing steps. A washing step may be accomplished by methods known to those in the art, such as by using a semi-automated “flowthrough” centrifuge (for example, the Cobe 2991 cell processor, the Baxter CytoMate, or the Haemonetics Cell Saver 5) according to the manufacturer's instructions.

In some examples, T cells may be isolated from peripheral blood lymphocytes by lysing the red blood cells and depleting the monocytes, for example, by centrifugation through a PERCOLL™ gradient or by counterflow centrifugal elutriation.

Exemplary T cell populations include naïve T cells, T helper cells (T_{H} cells), terminally differentiated effector T cells (T_{eff} cells), effector memory T cells (Tₑₘ cells), central memory T cells (Tₑₘ cells), cytotoxic T cells (CTLs) and regulatory T cells (T_{reg} cells).

In some examples, a specific subpopulation of T cells, such as CD3+, CD28+, CD4+, CD8+, CD45RA+, and CD45RO+ T cells, can be further isolated by positive or negative selection techniques.

In some examples, the T cell subpopulations are isolated by positive selection e.g., before or after introduction of the ddRNAi construct, CAR construct, DNA construct, and/or expression vector of the disclosure. For example, the T cells isolated from the blood of a subject can be incubated with an antibody that specifically recognizes a particular cell-surface protein under condition suitable for antibody binding. In some examples, the antibody may be conjugated to a fluorescent molecule, e.g., FITC, and the T cells are sorted using flow cytometry.

In one example, a subpopulation of T-cells which are resistant to an immunosuppressant may be isolated by culturing the T-cell in the presence of an immunosuppressant and selecting those T-cells which survive.

Methods of preparing T-cells as described herein can include more than one selection step. For example, in addition to positive selection described above, further enrichment of a T cell population by negative selection can be accomplished, e.g., with a combination of antibodies directed to surface markers unique to the negatively selected cells, for example regulatory T cells or tumor cells. One such method is cell sorting and/or selection via flow cytometry that uses a cocktail of monoclonal antibodies directed to cell surface markers present on the cells negatively selected. Such antibodies include anti-GITR, anti-CD25, or anti-tumor antigen antibodies.

In some examples, the collection of blood samples or apheresis product from a subject is made at a time period prior to when the expanded cells might be needed. As such, the source of the cells to be expanded can be collected at any time point necessary, and desired T cells may be isolated and frozen for later use in, e.g., T cell therapy for any number of diseases or conditions that would benefit from such T cell therapy.

A T cell produced in accordance with the methods described herein can be allogeneic e.g., an allogeneic T cell lacking expression of a functional TCR and/or expressing a CAR.

The methods may further comprise HLA typing the T-cell(s) e.g., as described herein.

For example, the methods are performed ex vivo.

In some examples, the method include first stimulating cell growth, e.g., T cell growth, proliferation, and/or activation, followed by transduction of the activated cells, and expansion in culture to numbers sufficient for clinical applications.

### Banking of T Cells

In one example, a plurality of T cells described herein, or compositions comprising same, are in a bank. In one example, the T-cells in the bank comprise a ddRNAi construct of the disclosure and possess a non-functional TCR. In addition, the T-cells in the bank may be CAR-T cells of the disclosure.

In accordance with this example, the T cells of the disclosure may be “banked” for future use, at a cell bank or depository or storage facility, or any place where such as cells are kept cryopreserved, e.g., in liquid nitrogen, for safekeeping. Furthermore, appropriate computer systems can be used for data processing, to maintain records relating to donor information and to ensure rapid and efficient retrieval of cells from the storage repositories.

In one example, each of the storage containers (e.g., bags or tubes) can be tagged with positive identification based on a distinctive property associated with the donor, lines or cell type, prior to storing in a bank according to the disclosure. For example, DNA genetic fingerprint and HLA typing may be used with secured identification mechanism such as acceptable methods using microchips, magnetic strip, and/or bar code labels. This identification step may be included in the banking process.

In one example, at least one of the HLA alleles in the T cells in each composition in the bank has been identified. In one example, the HLA is a HLA-DR allele.

At the time of use, only the required storage unit is retrieved, the number of units necessary to fulfil a desired dosage being selectable. Certain diseases may require cell therapy that includes a series of repeated treatments. The population of cells may be extracted from the bank and increased by cellular expansion before preparation of the pharmaceutical composition and administration to the subject.

Suitable cells for use in the preparation of T-cells with non-functional TCR as described herein, CAR-T cells as described herein, and composition comprising same, may be obtained from existing cell banks, or may be directly collected from one or more donor subjects and later banked. In one example, cells are collected from healthy subjects. For example, cells from tissues that are non-essential to the subject may also be appropriate as they reduce the risk of induction of autoimmune disease.

Standards for donor selection may include one or more of the following considerations prior to collection, such as (a) absence of specific disease; (b) specific or general diseases; (c) parameters of the donor relating to certain diseases, for example a certain age, certain physical conditions and/or symptoms, with respect to certain specific diseases, with respect to certain prior treatment history and/or preventive treatment, etc.; (d) whether the donor fits into one or more established statistical and/or demographic models or profiles (e.g., statistically unlikely to acquire certain diseases); and (e) whether the donor is in a certain acceptable health condition as perceived based on prevailing medical practices, etc.

In one example, the cells are collected by apheresis from donor's peripheral blood, processed (to optimise the quantity and quality of the collected cells) and, optionally cryogenically preserved or maintained in culture under suitable conditions. In one example, the donor is a stem cell donor. For example, the cells are collected by apheresis as part of the stem cell donation. In one example, the cells are collected after administration of G-CSF to the donor alone or in combination with chemotherapy or a stem cell mobilising agent. In one example, the cells are collected by bone marrow harvest.

In one example, the cells are collected by apheresis from the donor's peripheral blood or from the bone marrow by marrow harvest and are used for the preparation of the composition if the number of cells collected exceeds the number required for the purposes of stem cell transplantation. For example, the cells collected for the preparation of the composition are in excess of the cells required for stem cell transplantation.

The collected cells can be aliquoted into defined dosage fractions. The cells may be stored under any appropriate conditions, such as in culture or in a cryopreserved state.

Methods of cell storage will be apparent to the skilled person. For example, cryopreservation of cells can be achieved using a variety of cryoprotecting agents, such as DMSO.

T-cells of the disclosure may be cryopreserved for adoptive T cell transfer. For example, a freezing mix containing 40% saline, 40% Albumex20 and 20% DMSO is prepared. The saline is added to the DMSO and chilled before adding the Albumex20. The freezing mix is kept chilled until required.

The cells for cryopreservation are resuspended, pooled and mixed thoroughly. The cells are counted using a haemocytometer and the cell concentration and total cell viability is determined.

The cells are spun at 1400 rpm for 5 mins and 10 mls of the supernatant is removed for sterility and mycoplasma testing. The remaining supernatant is discarded.

The cells are washed with up to 200 ml of 0.9% saline supplemented with Albumex20 and spun at 1400 rpm for 5 mins.

The cells are resuspended in 0.9% saline at a concentration of 2×10⁷ cells/ml. For cryopreserving the T cells the maximum volume of cells to be added per bag is to be calculated using the formula: Maximum volume per bag (mL)=Max number of cells required per bag/1×10⁷ per ml.

The number of bags and quality assurance samples to be cryopreserved is determined. An equal volume of freezing mix is added to the T lymphocyte suspension and mixed. The required volume of cells is transferred into cryopreservation bags and/or vials. The bags and vials are immediately placed into pre-cooled rate controlled freezers to begin cryopreservation.

Phenotyping of cells for use in therapy and bankin!

In one example, the T-cells of the present disclosure are HLA-allele phenotyped. For example, the cells are partially HLA-allele phenotyped.

In one example, the cells have alleles selected from major HLA, such as any Class I, II or III HLA, minor HLA, and non-polymorphic alleles, such as any member of the CD1 family members.

Major HLA alleles may more specifically be selected from any class I HLA such as HLA-A1, HLA-A2, HLA-A3, HLA-A24, HLA-A11, HLA-A28, HLA-A29, HLA-A32, HLA-B15, HLA-B5, HLA-B7, HLA-B8, HLA-B12, HLA-B14, HLA-B18, HLA-B35, HLA-B40, HLA-C group 1, HLA-C group 2 for example, any class II HLA-DPB9, HLA-DPB11, HLA-DPB35, HLA-DPB55, HLA-DPB56, HLA-DPB69 HLA-DPB84 HLA-DPB 87, HLA-DRB1, HLA-DQA1, HLA-DQB1, or any class III HLA. The knowledge of a HLA phenotype can facilitate subsequent selection of cells for the preparation of the composition of the present disclosure.

In one example, at least one class II HLA is phenotyped. For example, at least one of HLA-DR, HLA-DP or HLA-DQ is phenotyped.

In one example, at least one HLA-allele in the cells of the present disclosure is matched to at least one HLA-allele in the subject to which the composition is administered. For example, at least one class II HLA is matched. For example, at least one of HLA-DR, HLA-DP and HLA-DQ is matched.

In one example, the HLA allele is HLA-DR. For example, the phenotype of HLA-DR in the cells of the present disclosure is matched to an HLA-DR allele in the subjection to which the composition is administered. In one example, the method of treating a subject comprises determining an HLA allele in the subject, matching the HLA allele to an HLA allele in T cells in a composition in the bank and administering to the subject a composition comprising T cells having the same HLA allele as that in the subject.

### Therapeutic Methods

The present disclosure also contemplates the use of the T-cells (i.e., CAR-T cells) comprising the DNA construct of the disclosure (e.g., expressing a CAR as described herein) in therapy.

In one example, the present disclosure provides a method of treating or preventing a disease or condition selected from cancer, graft versus host disease, infection, one or more autoimmune disorders, transplantation rejection, and radiation sickness in an individual in need thereof, comprising administering to the individual a CAR-T cell as described herein or a formulation comprising same.

In one example, the present disclosure provides a method of treating a disease or condition associated with expression of a cancer associated antigen (or tumor antigen) as described herein. In one example, the disease to be treated is cancer. For example, the method may comprise administering to a subject a T-cell of the disclosure which has been engineered to express a CAR which binds specifically to the cancer associated antigen. When the CAR-T cell of the disclosure contacts a tumor cell with at least one cancer associated antigen expressed on its surface, the CART targets the tumor cell and growth of the tumor is inhibited.

In one example, the present disclosure provides a method of inhibiting growth of a cancer, comprising contacting a cancer cell with a CAR-T cell described herein. In accordance with this example, the CAR-T cell is activated in response to the antigen expressed on the surface of the cancer cell, targets the cancer cell and inhibits its growth.

As used herein, the term “cancer” is intended to include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one example, the cancer is a melanoma, e.g., an advanced stage melanoma. Metastatic lesions of the aforementioned cancers can also be treated or prevented using the methods and compositions of the disclosure. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

Exemplary cancers which may be treated using the methods of the disclosure include cancers typically responsive to immunotherapy. Non-limiting examples of cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer).

The present methods may be particularly useful for treating hematological cancer conditions. Hematological cancer conditions are the types of cancer such as leukemia and malignant lymphoproliferative conditions that affect blood, bone marrow and the lymphatic system. Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Accordingly, in one example, the method of treating cancer as described herein is a method of treating a hematologic cancer including, but is not limited to hematological cancer which is a leukemia or a lymphoma. In one example, the CAR-T cells of the disclosure may be used to treat cancers and malignancies such as, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocyte leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplasia syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like.

In one example, the present disclosure provides methods for inhibiting the proliferation or reducing the population of cancer cells expressing a cancer associate antigen as described herein, the methods comprising contacting a cell expressing a cancer associated antigen with a CAR-T cell that binds to the a cancer associated antigen as described herein. In certain examples, the CAR-T cells of the disclosure reduce the quantity, number, amount or percentage of cells and/or cancer cells by at least 25%, at least 30%, at least 40%, at least 50%, at least 65%, at least 75%, at least 85%, at least 95%, or at least 99% in the subject. In one example, the subject is a human.

Additionally, refractory or recurrent malignancies can be treated using the CAR-T cells and formulations comprising same as described herein. As used herein, the term “refractory” refers to a disease, e.g., cancer, that does not respond to a treatment. In some examples, a refractory cancer can be resistant to a treatment before or at the beginning of the treatment. In other examples, the refractory cancer can become resistant during a treatment. In one example, the treatment is chemotherapy, hematopoietic stem cell transplantation or immunoablation. For example, the subject is undergoing or about to commence or has completed chemotherapy and/or hematopoietic stem cell transplantation and/or immunoablation therapy.

In accordance with one example in which a method of treating or preventing graft versus host disease or transplantation rejection is provided, the subject to be treated may be about to receive or has received transplantation of a solid organ such as a kidney, liver, pancreas, pancreatic islets, heart, lungs, small bowel or other solid organ.

In accordance with an another example, a subject to be treated with a method of the disclosure is receiving or has received immunosuppressive drug treatment or antibody treatment or soluble receptor treatment or another immunomodulating treatment for a disease such as, but not limited to, inflammatory bowel disease, rheumatoid arthritis, multiple sclerosis, hepatitis, glomerulonephritis and kidney failure, cancer, lymphoma, leukemia, myelodysplasia, myeloma.

In accordance with an another example, a subject to be treated with a method of the disclosure, the subject to be treated has inherited or been born with a deficiency of the immune system such as, but not limited to, severe combined immune deficiency, common variable immunodeficiency, alymphocytosis, Wiskott Aldrich syndrome, ataxia telangiectasia, di George syndrome, leucocyte adhesion defects, immunoglobulin deficiency.

In accordance with an another example, a subject to be treated with a method of the disclosure, the subject has an acquired immunodeficiency through infection with the human immunodeficiency virus or another pathogenic organism that has led to incompetence of the immune system.

In one example, the present disclosure provides a method of treating a disease or condition associated with expression of a viral antigen as described herein, comprising administering to the individual a CAR-T cell as described herein or a formulation comprising same. For example, the CAR-T cell expresses a CAR which binds specifically to the viral antigen or viral-induced antigen. In one example, administration of the T-cell to a subject confers a therapeutic immune response against the virus. In one example, administration of the T cell to a subject confers a protective immune response against a virus. For example, the virus antigen or viral-induced antigen may be from a virus selected from the group consisting of Human cytomegalovirus (HCMV), Human immunodeficiency virus (HIV), Epstein-Barr virus (EBV), adenovirus (AdV), varicella zoster virus (VZV), influenza and BK virus (BKV), John Cunningham (JC) virus, respiratory syncytial virus (RSV), parainfluenzae, rhinovirus, human metapneumovirus, herpes simplex virus (HSV) 1, HSV II, human herpes virus (HHV) 6, HHV 8, Hepatitis A virus, Hepatitis B virus (HBV), Hepatitis C virus (HCV), hepatitis E virus, rotavirus, papillomavirus, parvovirus Ebola virus, zika virus, a hantavirus and vesicular stomatitis virus (VSV).

The methods of the disclosure may comprise infusing an individual to be treated with CAR-T cells of the disclosure which have been genetically modified to express a particular CAR. The infused cells are able to kill the diseased cells e.g., cancer cells or virus infected cell, in the recipient. Unlike antibody therapies, CAR-modified T cells, are able to replicate in vivo resulting in long-term persistence that can lead to sustained treatment e.g., tumor control. In various aspects, T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cells to the patient.

The present disclosure also contemplates a type of cellular therapy where T-cells with non-functional TCR as described herein are further modified e.g., by in vitro transcription of RNA from a CAR construct of the disclosure, to transiently express a CAR, after which time the CAR-T cell is infused to a recipient in need thereof. The infused cell is able to kill the diseased cells e.g., cancer cells, in the recipient. However, in contrast to an example in which a T-cell has been stably transfected or transduced with a CAR construct of the disclosure, T cells administered to the patient in accordance with this example are present for less than one month, e.g., three weeks, two weeks, one week, after administration of the T cells to the patient.

In accordance with one method of treatment, T-cells are isolated from a mammal (e.g., a human) and genetically modified (i.e., transduced or transfected in vitro) with a vector expressing a ddRNAi construct and a CAR construct as disclosed herein e.g., a vector comprising a DNA construct of the disclosure. The CAR-T cells can be administered to a mammalian recipient to provide a therapeutic benefit. The mammalian recipient may be a human and the CAR-T cells can be autologous with respect to the recipient.

Alternatively, the cells can be allogeneic or syngeneic with respect to the recipient. In accordance with this example, the T-cells may have been HLA-typed to determine compatibility with the recipient.

Generally, the CAR-T cells as described herein may be utilized in the treatment and prevention of diseases that arise in individuals who are immunocompromised. In particular, the CAR-T cells of the disclosure are used in the treatment of diseases, disorders and conditions associated with expression of cancer associate antigens as described herein. In certain examples, the CAR-T cells of the disclosure are used in the treatment of patients at risk for developing diseases, disorders and conditions associated with expression of a cancer associate antigen as described herein. Thus, the present disclosure provides methods for the treatment or prevention of diseases, disorders and conditions associated with expression of a cancer-associated antigen as described herein comprising administering to a subject in need thereof, a therapeutically effective amount of the CAR-T cell or formulation comprising same as described herein.

The CAR-T cell of the present disclosure may be administered either alone, or as a pharmaceutical composition in combination with diluents and/or with other components such as IL-2 or other cytokines or cell populations.

### Combination Therapy

The CAR-T cells and formulations comprising same as described herein may be used in combination with other known agents and therapies for treatment of a particular disease or condition. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In one example, the delivery of one treatment is still occurring when the delivery of the second begins, so that there is overlap in terms of administration. This is sometimes referred to herein as “simultaneous” or “concurrent delivery”. In other examples, the delivery of one treatment ends before the delivery of the other treatment begins. In some examples of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some examples, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In one example, the CAR-T cells described herein or formulation comprising same and the at least one additional therapeutic agent can be administered simultaneously, in the same or in separate compositions, or sequentially. For sequential administration, the CAR-T cell described herein and the additional agent can be administered in either order.

The CAR-T cell therapy and/or other therapeutic agents, procedures or modalities can be administered during periods of active disorder, or during a period of remission or less active disease. The CAR-T cell therapy can be administered before another treatment, concurrently with the treatment, post-treatment, or during remission of the disorder.

When administered in combination, the CAR-T cell therapy and the additional agent (e.g., second or third agent), or all, can be administered in an amount or dose that is higher, lower or the same than the amount or dosage of each agent used individually, e.g., as a monotherapy. In certain examples, the administered amount or dosage of the CAR-T cell therapy, the additional agent (e.g., second or third agent), or all, is lower (e.g., at least 20%, at least 30%, at least 40%, or at least 50%) than the amount or dosage of each agent used individually, e.g., as a monotherapy. In other examples, the amount or dosage of the CAR-T cell therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In accordance with an example in which the disease or condition to be treated is cancer, the additional therapeutic agent or treatment regimen may include, but is not limited to, surgery, chemotherapy, radiation, immunosuppressive agents, antibodies, immunoablative agents, steroids, and irradiation.

### Dosage and Administration

The dosage ranges for the administration of the CAR-T cell formulations of the disclosure are those large enough to produce the desired effect. For example, the formulation should comprise an amount of the CAR-T cells sufficient to confer a therapeutic or protective immune response in the subject.

The dosage should not be so large as to cause adverse side effects, such as hyper viscosity syndromes, pulmonary edema, congestive heart failure, and the like. Generally, the dosage will vary with the age, condition, sex and extent of the disease in the subject and can be determined by one of skill in the art. The dosage can be adjusted by the individual physician in the event of any complication. Dosage can vary from about 1×10³ cells/kg to about 1×10¹⁰ cells/kg. For example about 1×10³ cell/kg to about 1×10⁴ cells/kg, or about 1×10⁴ cell/kg to about 1×10⁵, or about 1×10⁵ cell/kg to about 1×10⁶, or about 1×10⁶ cell/kg to about 1×10⁷, or about 1×10⁷ cell/kg to about 1×10⁸, or about 1×10⁸ cell/kg to about 1×10⁹, or about 1×10⁹ cell/kg to about 1×10¹⁰. Dosage can vary from about 1×10⁵ cells/m² to about 1×10¹⁰ cells/m². For example, about 1×10⁵ cells/m² to about 1×10⁶ cells/m², or about 1×10⁶ cells/m² to about 1×10⁷ cells/m², or about 1×10⁷ cells/m² to about 1×10⁸ cells/m², or about 1×10⁸ cells/m² to about 1×10⁹ cells/m², or about 1×10⁹ cells/m² to about 1×10¹⁰ cells/m². For example, about 1×10⁷ cells/m², or about 2×10⁷ cells/m², or about 3×10⁷ cells/m², or about 4×10⁷ cells/m² or about 5×10⁷ cells/m². In one example, the dosage may be administered in one or more dose administrations. In one example, the dosage can be repeated at least once. For example, the dosage is repeated at intervals depending on the immune state of the subject and the response to the previous infusion. In this regard, the repeat dosage(s) need not be the same as previous dosage(s), e.g., it could be increased or decreased.

In one example, the formulation is administered intravenously.

In the case of a subject that is not adequately responding to treatment, multiple doses may be administered. Alternatively, or in addition, increasing doses may be administered.

### Examples

### Example 1—Design and Screening of shRNA and shmiR Targeting TCR Subunits

To define constructs capable of silencing expression of TCR components, shRNAs were designed to target regions of TCR-α, TCR-β, CD3-ε, CD3-δ and CD3-γ mRNAs. Target regions were selected from regions of absolute sequence conservation between the human, mouse and macaque mRNA sequences, since the use of conserved sequences potentially simplifies pre-clinical testing of construct safety and efficacy. Sequences representing potential targets for design of shRNA and shmiR constructs were identified from the mRNA sequences of T cell Receptor (TCR) subunits: TCR-α (SEQ ID NOs:180-182), TCR-β (SEQ ID NOs:183-185), CD3-6 (SEQ ID NOs:186-188), CD3-γ (SEQ ID NOs: 189-191) and CD3-ε (SEQ ID NOs: 192-194). Publicly available algorithms (including Ambion, Promega, Invitrogen, Origene and MWG) were used to select sequences. Sequences targeting the TCR-α and TCR-β subunits were only designed against the constant region of those subunits.

Six shRNAs targeting TCR-α, nine shRNAs targeting TCR-β, thirteen shRNAs targeting CD3-ε, thirteen shRNAS targeting CD3-δ and seven shRNAs targeting CD3-γ were screened for activity. The sequences of effector and effector complements for these shRNAs are listed in Table 1. The silencing activity of these constructs were assayed with dual luciferase assays using sensor constructs where shRNA target sites were cloned into the 3′ UTR of a luciferase reporter construct. The activities of both effector and effector complement sequences were determined using individual sensor constructs, where target sites were cloned respectively in either the sense or antisense orientation. Construct activities and strand specificities varied considerably. Three effector sequences for TCR-α, three for TCR-β, three for CD3-ε, four for CD3-δ and two for CD3-γ were selected for further characterisation.

The selected effector/effector complement sequences were then used to construct shmiR expressing constructs. In some instances, variants of individual shmiR constructs were designed and tested; for these, effector and effector complement sequences were moved a few base pairs upstream or downstream of the original shRNA targeting sites, in an attempt to yield constructs with enhanced activity and/or strand specificity. The activities and strand specificities of these shmiR constructs were determined using dual luciferase assays and strand specific sensor constructs as described below. The relative activities of these constructs were then determined using “hyperfunctional assays”. In such experiments, varying amounts of shmiR constructs were titrated against a constant amount of sensor construct and luciferase knockdown quantified; constructs that showed strong knockdown with the lowest amounts of DNA were considered to be the most active. The activities of these constructs were also determined against the endogenous gene targets by assaying mRNA knockdown for individual target genes in transfected Jurkat cells using qRT PCR assays. In addition, target protein knockdown were assayed using Western blots in transfected Jurkat cells.

These data were then used to select individual shmiRs, listed in Tables 2 and 3, for subsequent analyses.

### Example 2—Design of shmiRs Targeting the Endogenous T Cell Receptor

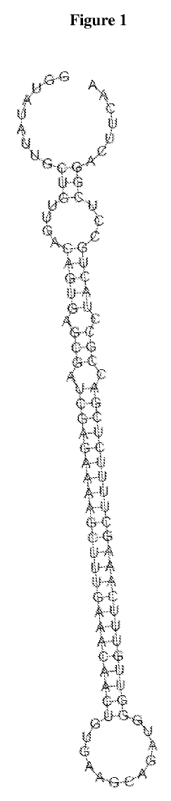

Sequences encoding the shRNAs selected in Example 1 were incorporated into a pre-miRNA scaffold in order to create short-hairpin microRNAs (shmiRs), comprising a 5′ flanking region (SEQ ID NO: 98), a sense strand, a stem/loop sequence (SEQ ID NO: 97), an anti-sense strand, and a 3′ flanking region (SEQ ID NO: 99). The predicted secondary structure of a representative shmiR is shown in FIG. 1. The effector sequences (antisense strand) and complement sequences (sense strand) for each of the candidate shmiRs are presented in Table 2 and the full shmiR sequences are shown in Table 3.

TABLE 1
shRNA effector and effector complement sequences

| | | | Effector complement | |
|---|---|---|---|---|
| shRNA ID | Effector sequence (5′-3′) | SEQ ID NO | sequence (5′-3′) | SEQ ID NO |
| TCR-α 1 | UCUGUUUCAAAGCUUUUCUCG | SEQ ID NO: 1 | CGAGAAAAGCUUUGAAACAGA | SEQ ID NO: 2 |
| TCR-α 2 | UCGUAUCUGUUUCAAAGCUUU | SEQ ID NO: 3 | AAAGCUUUGAAACAGAUACGA | SEQ ID NO: 4 |
| TCR-α 3 | UAGGUUCGUAUCUGUUUCAAA | SEQ ID NO: 5 | UUUGAAACAGAUACGAACCUA | SEQ ID NO: 6 |
| TCR-α 4 | AAGUUUAGGUUCGUAUCUGUU | SEQ ID NO: 7 | AACAGAUACGAACCUAAACUU | SEQ ID NO: 8 |
| TCR-α 5 | UUUGAAAGUUUAGGUUCGUAU | SEQ ID NO: 9 | AUACGAACCUAAACUUUCAAA | SEQ ID NO: 10 |
| TCR-α 6 | AGGUUUUGAAAGUUUAGGUUC | SEQ ID NO: 11 | GAACCUAAACUUUCAAAACCU | SEQ ID NO: 12 |
| TCR-β 1 | ACCAGCUCAGCUCCACGUGGU | SEQ ID NO: 13 | ACCACGUGGAGCUGAGCUGGU | SEQ ID NO: 14 |
| TCR-β 2 | CCAUUCACCCACCAGCUCAGC | SEQ ID NO: 15 | GCUGAGCUGGUGGGUGAAUGG | SEQ ID NO: 16 |
| TCR-β 3 | GACCCCACUGUGCACCUCCUU | SEQ ID NO: 17 | AAGGAGGUGCACAGUGGGGUC | SEQ ID NO: 18 |
| TCR-β 4 | GUGCUGACCCCACUGUGCACC | SEQ ID NO: 19 | GGUGCACAGUGGGGUCAGCAC | SEQ ID NO: 20 |
| TCR-β 5 | CAGGCGGCUGCUCAGGCAGUA | SEQ ID NO: 21 | UACUGCCUGAGCAGCCGCCUG | SEQ ID NO: 22 |
| TCR-β 6 | GAGACCCUCAGGCGGCUGCUC | SEQ ID NO: 23 | GAGCAGCCGCCUGAGGGUCUC | SEQ ID NO: 24 |
| TCR-β 7 | GACUUGACAGCGGAAGUGGUU | SEQ ID NO: 25 | AACCACUUCCGCUGUCAAGUC | SEQ ID NO: 26 |
| TCR-β 8 | UCAGGCGGCUGCUCAGGCAGU | SEQ ID NO: 27 | ACUGCCUGAGCAGCCGCCUGA | SEQ ID NO: 28 |
| TCR-β 9 | UCACCCACCAGCUCAGCUCCA | SEQ ID NO: 29 | UGGAGCUGAGCUGGUGGGUGA | SEQ ID NO: 30 |
| CD3-ϵ 1 | CCUUUCUAUUCUUGCUCCAGU | SEQ ID NO: 31 | ACUGGAGCAAGAAUAGAAAGG | SEQ ID NO: 32 |
| CD3-ϵ 2 | CACAGGCUUGGCCUUGGCCUU | SEQ ID NO: 33 | AAGGCCAAGGCCAAGCCUGUG | SEQ ID NO: 34 |
| CD3-ϵ 3 | GGGUUGGGAACAGGUGGUGGC | SEQ ID NO: 35 | GCCACCACCUGUUCCCAACCC | SEQ ID NO: 36 |
| CD3-ϵ 4 | CUCAUAGUCUGGGUUGGGAAC | SEQ ID NO: 37 | GUUCCCAACCCAGACUAUGAG | SEQ ID NO: 38 |
| CD3-ϵ 5 | GGAUGGGCUCAUAGUCUGGGU | SEQ ID NO: 39 | ACCCAGACUAUGAGCCCAUCC | SEQ ID NO: 40 |
| CD3-ϵ 6 | AGAAUACAGGUCCCGCUGGCC | SEQ ID NO: 41 | GGCCAGCGGGACCUGUAUUCU | SEQ ID NO: 42 |
| CD3-ϵ 7 | CUCUGAUUCAGGCCAGAAUAC | SEQ ID NO: 43 | GUAUUCUGGCCUGAAUCAGAG | SEQ ID NO: 44 |
| CD3-ϵ 8 | UAGUCUGGGUUGGGAACAGGU | SEQ ID NO: 45 | ACCUGUUCCCAACCCAGACUA | SEQ ID NO: 46 |
| CD3-ϵ 9 | UUCCGGAUGGGCUCAUAGUCU | SEQ ID NO: 47 | AGACUAUGAGCCCAUCCGGAA | SEQ ID NO: 48 |
| CD3-ϵ 10 | UCAGGCCAGAAUACAGGUCCC | SEQ ID NO: 49 | GGGACCUGUAUUCUGGCCUGA | SEQ ID NO: 50 |
| CD3-ϵ 11 | AUUCAGGCCAGAAUACAGGUC | SEQ ID NO: 51 | GACCUGUAUUCUGGCCUGAAU | SEQ ID NO: 52 |
| CD3-ϵ 12 | GAUUCAGGCCAGAAUACAGGU | SEQ ID NO: 53 | ACCUGUAUUCUGGCCUGAAUC | SEQ ID NO: 54 |
| CD3-ϵ 13 | UUCUUCAUUACCAUCUUGCCC | SEQ ID NO: 55 | GGGCAAGAUGGUAAUGAAGAA | SEQ ID NO: 56 |
| CD3-δ 1 | GUAUCUUGAAGGGGCUCACUU | SEQ ID NO: 57 | AAGUGAGCCCCUUCAAGAUAC | SEQ ID NO: 58 |
| CD3-δ 2 | AUAUAUUCCUCGUGGGUCCAG | SEQ ID NO: 59 | CUGGACCCACGAGGAAUAUAU | SEQ ID NO: 60 |
| CD3-δ 3 | UCCCAAAGCAAGGAGCAGAGU | SEQ ID NO: 61 | ACUCUGCUCCUUGCUUUGGGA | SEQ ID NO: 62 |
| CD3-δ 4 | AAAGCAGAAGACUCCCAAAGC | SEQ ID NO: 63 | GCUUUGGGAGUCUUCUGCUUU | SEQ ID NO: 64 |
| CD3-δ 5 | GUCUCAUGUCCAGCAAAGCAG | SEQ ID NO: 65 | CUGCUUUGCUGGACAUGAGAC | SEQ ID NO: 66 |
| CD3-δ 6 | AUUCCUCGUGGGUCCAGGAUG | SEQ ID NO: 67 | CAUCCUGGACCCACGAGGAAU | SEQ ID NO: 68 |
| CD3-δ 7 | CCUAUAUAUUCCUCGUGGGUC | SEQ ID NO: 69 | GACCCACGAGGAAUAUAUAGG | SEQ ID NO: 70 |
| CD3-δ 8 | CACCUAUAUAUUCCUCGUGGG | SEQ ID NO: 71 | CCCACGAGGAAUAUAUAGGUG | SEQ ID NO: 72 |
| CD3-δ 9 | CAAGGAGCAGAGUGGCAAUGA | SEQ ID NO: 73 | UCAUUGCCACUCUGCUCCUUG | SEQ ID NO: 74 |
| CD3-δ 10 | CUGAGCAUCAUCUCGAUCUCG | SEQ ID NO: 75 | CGAGAUCGAGAUGAUGCUCAG | SEQ ID NO: 76 |
| CD3-δ 11 | AUAUCUGUCCCAUUACACCUA | SEQ ID NO: 77 | UAGGUGUAAUGGGACAGAUAU | SEQ ID NO: 78 |
| CD3-δ 12 | UAUAUCUGUCCCAUUACACCU | SEQ ID NO: 79 | AGGUGUAAUGGGACAGAUAUA | SEQ ID NO: 80 |
| CD3-δ 13 | AAUGACAUCAGUGACAAUGAU | SEQ ID NO: 81 | AUCAUUGUCACUGAUGUCAUU | SEQ ID NO: 82 |
| CD3-γ 1 | CAAGUGUAUUACAGAAUGUGU | SEQ ID NO: 83 | ACACAUUCUGUAAUACACUUG | SEQ ID NO: 84 |
| CD3-γ 2 | GGACAGGAUGGAGUUCGCCAG | SEQ ID NO: 85 | CUGGCGAACUCCAUCCUGUCC | SEQ ID NO: 86 |
| CD3-γ 3 | GUUCGCCAGUCGAGAGCUUCA | SEQ ID NO: 87 | UGAAGCUCUCGACUGGCGAAC | SEQ ID NO: 88 |
| CD3-γ 4 | CAGACAAGCAGACUCUGUUGC | SEQ ID NO: 89 | GCAACAGAGUCUGCUUGUCUG | SEQ ID NO: 90 |
| CD3-γ 5 | ACCAGCCCCUCAAGGAUCGAG | SEQ ID NO: 91 | CUCGAUCCUUGAGGGGCUGGU | SEQ ID NO: 92 |
| CD3-γ 6 | GAGCUUCAGACAAGCAGACUC | SEQ ID NO: 93 | GAGTCTGCTTGTCTGAAGCTC | SEQ ID NO: 94 |
| CD3-γ 7 | UCCAAGUGUAUUACAGAAUGU | SEQ ID NO: 95 | ACATTCTGTAATACACTTGGA | SEQ ID NO: 96 |

TABLE 2
shmiR effector and effector complement sequences

| | | | Effector complement | |
|---|---|---|---|---|
| shmiR ID | Effector sequence (5′-3′) | SEQ ID NO | sequence (5′-3′) | SEQ ID NO |
| shmiR-TCR-α_1 | UGUUUCAAAGCUUUUCUCGAC | SEQ ID NO: 100 | UCGAGAAAAGCUUUGAAACA | SEQ ID NO: 101 |
| shmiR-TCR-α_2 | UUUCAAAGCUUUUCUCGACCA | SEQ ID NO: 102 | GGUCGAGAAAAGCUUUGAAA | SEQ ID NO: 103 |
| shmiR-TCR-α_3 | AAGUUUAGGUUCGUAUCUGUU | SEQ ID NO: 104 | ACAGAUACGAACCUAAACUU | SEQ ID NO: 105 |
| shmiR-TCR-α_4 | UUUGAAAGUUUAGGUUCGUAU | SEQ ID NO: 106 | UACGAACCUAAACUUUCAAA | SEQ ID NO: 107 |
| shmiR-TCR-β_1 | CCAUUCACCCACCAGCUCAGC | SEQ ID NO: 108 | CUGAGCUGGUGGGUGAAUGG | SEQ ID NO: 109 |
| shmiR-TCR-β_2 | GUGGCCGAGACCCUCAGGCGG | SEQ ID NO: 110 | CGCCUGAGGGUCUCGGCCAC | SEQ ID NO: 111 |
| shmiR-TCR-β_3 | ACUGGACUUGACAGCGGAAGU | SEQ ID NO: 112 | CUUCCGCUGUCAAGUCCAGU | SEQ ID NO: 113 |
| shmiR-TCR-β_4 | CUUGACAGCGGAAGUGGUUGC | SEQ ID NO: 114 | CAACCACUUCCGCUGUCAAG | SEQ ID NO: 115 |
| shmiR-TCR-β_5 | UGACAGCGGAAGUGGUUGCGG | SEQ ID NO: 116 | CGCAACCACUUCCGCUGUCA | SEQ ID NO: 117 |
| shmiR-CD3-γ_1 | UGAAGCUCUCGACUGGCGAAC | SEQ ID NO: 118 | UUCGCCAGUCGAGAGCUUCA | SEQ ID NO: 119 |
| shmiR-CD3-γ_2 | ACAUUCUGUAAUACACUUGGA | SEQ ID NO: 120 | CCAAGUGUAUUACAGAAUGU | SEQ ID NO: 121 |
| shmiR-CD3-δ_1 | GUAUCUUGAAGGGGCUCACUU | SEQ ID NO: 122 | AGUGAGCCCCUUCAAGAUAC | SEQ ID NO: 123 |
| shmiR-CD3-δ_2 | AAAGCAGAAGACUCCCAAAGC | SEQ ID NO: 124 | CUUUGGGAGUCUUCUGCUUU | SEQ ID NO: 125 |
| shmiR-CD3-δ_3 | UGUACUGAGCAUCAUCUCGAU | SEQ ID NO: 126 | UCGAGAUGAUGCUCAGUACA | SEQ ID NO: 127 |
| shmiR-CD3-δ_4 | AAUGACAUCAGUGACAAUGAU | SEQ ID NO: 128 | UCAUUGUCACUGAUGUCAUU | SEQ ID NO: 129 |
| shmiR-CD3-ϵ_1 | AUUCAGGCCAGAAUACAGGUC | SEQ ID NO: 130 | ACCUGUAUUCUGGCCUGAAU | SEQ ID NO: 131 |
| shmiR-CD3-ϵ_2 | GAUUCAGGCCAGAAUACAGGU | SEQ ID NO: 132 | CCUGUAUUCUGGCCUGAAUC | SEQ ID NO: 133 |
| shmiR-CD3-ϵ_3 | UUCUUCAUUACCAUCUUGCCC | SEQ ID NO: 134 | GGCAAGAUGGUAAUGAAGAA | SEQ ID NO: 135 |

TABLE 3
shmiR sequences

| shmiR ID | shmiR sequences (5′-3′) | SEQ ID NO |
|---|---|---|
| shmiR-TCR-α_1 | GGUAUAUUGCUGUUGACAGUGAGCGAUCGAGAAAAGCUUUGAAACAACUGUGAAGCAGAUGG | SEQ ID NO: 136 |
| | GUUGUUUCAAAGCUUUUCUCGACCGCCUACUGCCUCGGACUUCAA | |
| shmiR-TCR-α_2 | GGUAUAUUGCUGUUGACAGUGAGCGAGGUCGAGAAAAGCUUUGAAAACUGUGAAGCAGAUGG | SEQ ID NO: 137 |
| | GUUUUCAAAGCUUUUCUCGACCACGCCUACUGCCUCGGACUUCAA | |
| shmiR-TCR-α_3 | GGUAUAUUGCUGUUGACAGUGAGCGUACAGAUACGAACCUAAACUUACUGUGAAGCAGAUGGG | SEQ ID NO: 138 |
| | UAAGUUUAGGUUCGUAUCUGUUCGCCUACUGCCUCGGACUUCAA | |
| shmiR-TCR-α_4 | GGUAUAUUGCUGUUGACAGUGAGCGUUACGAACCUAAACUUUCAAAACUGUGAAGCAGAUGGG | SEQ ID NO: 139 |
| | UUUUGAAAGUUUAGGUUCGUAUCGCCUACUGCCUCGGACUUCAA | |
| shmiR-TCR-β_1 | GGUAUAUUGCUGUUGACAGUGAGCGACUGAGCUGGUGGGUGAAUGGACUGUGAAGCAGAUGG | SEQ ID NO: 140 |
| | GUCCAUUCACCCACCAGCUCAGCCGCCUACUGCCUCGGACUUCAA | |
| shmiR-TCR-β_2 | GGUAUAUUGCUGUUGACAGUGAGCGACGCCUGAGGGUCUCGGCCACACUGUGAAGCAGAUGGG | SEQ ID NO: 141 |
| | UGUGGCCGAGACCCUCAGGCGGCGCCUACUGCCUCGGACUUCAA | |
| shmiR-TCR-β_3 | GGUAUAUUGCUGUUGACAGUGAGCGUCUUCCGCUGUCAAGUCCAGUACUGUGAAGCAGAUGGG | SEQ ID NO: 142 |
| | UACUGGACUUGACAGCGGAAGUCGCCUACUGCCUCGGACUUCAA | |
| shmiR-TCR-β_4 | GGUAUAUUGCUGUUGACAGUGAGCGACAACCACUUCCGCUGUCAAGACUGUGAAGCAGAUGGG | SEQ ID NO: 143 |
| | UCUUGACAGCGGAAGUGGUUGCCGCCUACUGCCUCGGACUUCAA | |
| shmiR-TCR-β_5 | GGUAUAUUGCUGUUGACAGUGAGCGACGCAACCACUUCCGCUGUCAACUGUGAAGCAGAUGGG | SEQ ID NO: 144 |
| | UUGACAGCGGAAGUGGUUGCGGCGCCUACUGCCUCGGACUUCAA | |
| shmiR-CD3-γ_1 | GGUAUAUUGCUGUUGACAGUGAGCGAUUCGCCAGUCGAGAGCUUCAACUGUGAAGCAGAUGGG | SEQ ID NO: 145 |
| | UUGAAGCUCUCGACUGGCGAACCGCCUACUGCCUCGGACUUCAA | |
| shmiR-CD3-γ_2 | GGUAUAUUGCUGUUGACAGUGAGCGACCAAGUGUAUUACAGAAUGUACUGUGAAGCAGAUGG | SEQ ID NO: 146 |
| | GUACAUUCUGUAAUACACUUGGACGCCUACUGCCUCGGACUUCAA | |
| shmiR-CD3-δ_1 | GGUAUAUUGCUGUUGACAGUGAGCGUAGUGAGCCCCUUCAAGAUACACUGUGAAGCAGAUGGG | SEQ ID NO: 147 |
| | UGUAUCUUGAAGGGGCUCACUUCGCCUACUGCCUCGGACUUCAA | |
| shmiR-CD3-δ_2 | GGUAUAUUGCUGUUGACAGUGAGCGACUUUGGGAGUCUUCUGCUUUACUGUGAAGCAGAUGG | SEQ ID NO: 148 |
| | GUAAAGCAGAAGACUCCCAAAGCCGCCUACUGCCUCGGACUUCAA | |
| shmiR-CD3-δ_3 | GGUAUAUUGCUGUUGACAGUGAGCGUUCGAGAUGAUGCUCAGUACAACUGUGAAGCAGAUGG | SEQ ID NO: 149 |
| | GUUGUACUGAGCAUCAUCUCGAUCGCCUACUGCCUCGGACUUCAA | |
| shmiR-CD3-δ_4 | GGUAUAUUGCUGUUGACAGUGAGCGUUCAUUGUCACUGAUGUCAUUACUGUGAAGCAGAUGG | SEQ ID NO: 150 |
| | GUAAUGACAUCAGUGACAAUGAUCGCCUACUGCCUCGGACUUCAA | |
| shmiR-CD3-ϵ_1 | GGUAUAUUGCUGUUGACAGUGAGCGAACCUGUAUUCUGGCCUGAAUACUGUGAAGCAGAUGGG | SEQ ID NO: 151 |
| | UAUUCAGGCCAGAAUACAGGUCCGCCUACUGCCUCGGACUUCAA | |
| shmiR-CD3-ϵ_2 | GGUAUAUUGCUGUUGACAGUGAGCGUCCUGUAUUCUGGCCUGAAUCACUGUGAAGCAGAUGGG | SEQ ID NO: 152 |
| | UGAUUCAGGCCAGAAUACAGGUCGCCUACUGCCUCGGACUUCAA | |
| shmiR-CD3-ϵ_3 | GGUAUAUUGCUGUUGACAGUGAGCGAGGCAAGAUGGUAAUGAAGAAACUGUGAAGCAGAUGG | SEQ ID NO: 153 |
| | GUUUCUUCAUUACCAUCUUGCCCCGCCUACUGCCUCGGACUUCAA | |

### Example 3-Downregulation of TCR Subunit Expression by Individual shmiRs

This example demonstrates the ability of the shmiRs to knockdown the endogenous expression of their targeted TCR subunit in vitro.

### Cells

Jurkat T cells were grown in RPMI medium (10% FCS, pen/strep) at 37 C, 5% CO2.

### Treatment

Cells were electroporated using the Neon Electroporation system (VOLTAGE=1350V, PULSE LENGTH=10, # OF PULSES=3) and transduced with individual shmiRs targeting the subunits of TCR. As a control, Jurkat T cells were transfected with the pSilencer plasmid expressing a non-targeting siRNA sequence.

Transduced cells were subsequently treated with anti-CD3 (5 ug/mL solution of anti-CD3e, OKT3) and anti-CD28 antibodies (soluble anti-CD28 to cells at 2 ug/mL) for 48 hours to activate the T cells. Following activation, the RNA was harvested and analysed by qPCR to measure the expression of the targeted TCR subunits and determine knockdown.

qPCR Analysis

RNA was harvested after 48 hours using Qiazol Reagent and RNA samples were quantified using a ND-1000 NanoDrop spectrophotometer (NanoDrop Technologies). cDNA was generated by reverse transcription using ABI ‘High Capacity cDNA Reverse Transcription Kit’ (Product No. 4368813) and Ambion ‘Superase Inhibitor’. cDNAs were used for quantitative PCR reaction using Taqman qPCR master mix in a total of 1 Oul reaction volumes. The PCR reactions were carried out as follows: 2 minutes at 50° C., 10 minutes at 95° C. followed by 40 cycles: 15 seconds at 95° C., 1 minute at 60° C. Primers were designed using GenScript TaqMan primer design tool (https://www.genscript.com/ssl-bin/app/primer).

The expression level of each mRNA was normalized to GAPDH. Expression levels were calculated according to the total copies as determine by a standard curve and converted to percent inhibition relative to the pSilencer control.

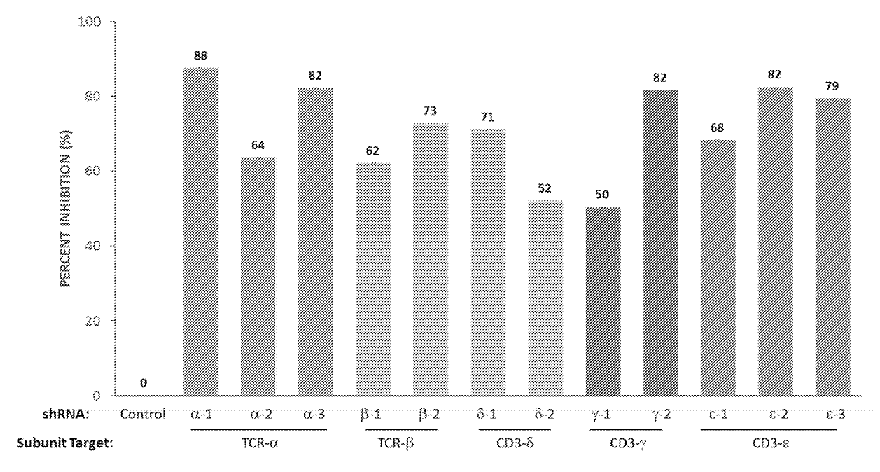

The resulting percent inhibition of the endogenous expression of TCR subunits in the Jurkat T cells by the shmiRs is presented in FIG. 2. As shown in FIG. 2, the shmiRs downregulated the expression of the TCR subunits with percent inhibition ranging between 50% to 88%.

### Example 4—Preparation of Triple shmiR Constructs Concomitantly Expressing Three shmiRs Targeting TCR Subunits

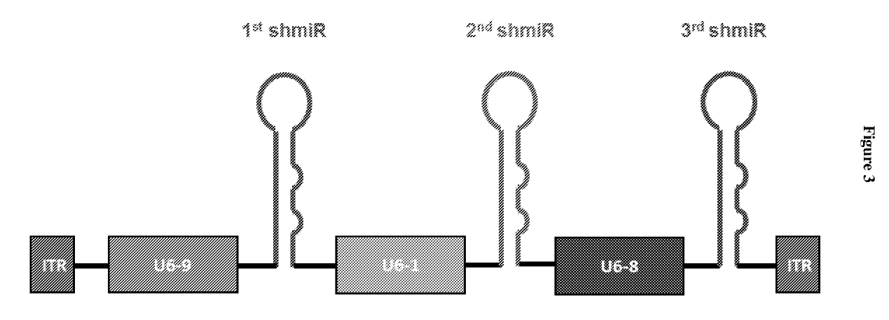

The leading candidate shmiRs, based on their inhibition of TCR subunit expression, were incorporated into lentiviral constructs concomitantly expressing three shmiRs. Each construct was comprised of: a 5′ lentiviral terminal repeat (LTR) sequence, the polymerase-III promoter U6-9 positioned upstream of the coding sequence of the first candidate shmiR, a U6-1 promoter upstream of the second shmiR coding sequence, a U6-8 promoter upstream of the third shmiR, followed by a 3′ LTR sequence. The shmiRs incorporated into each construct are indicated in Table 4 and an example of one such construct is illustrated in FIG. 3.

TABLE 4
Triple shmiR constructs

| Triple Construct ID | 1^{st} shmiR | 2^{nd} shmiR | 3^{rd} shmiR | SEQ ID NO: |
|---|---|---|---|---|
| pBL513 | shmiR-TCR-α_1 | shmiR-TCR-β_5 | shmiR-CD3-ε_3 | SEQ ID NO: 172 |
| pBL514 | shmiR-TCR-α_1 | shmiR-CD3-γ_2 | shmiR-CD3-ε_3 | SEQ ID NO: 173 |
| pBL515 | shmiR-TCR-α_1 | shmiR-CD3-δ_3 | shmiR-CD3-ε_3 | SEQ ID NO: 174 |
| pBL516 | shmiR-TCR-β_5 | shmiR-CD3-γ_2 | shmiR-CD3-ε_3 | SEQ ID NO: 175 |

### Example 5—Downregulation of TCR Surface Expression

This example demonstrates the ability of the triple shmiR constructs to simultaneously target different TCR subunits to prevent the expression and assembly of TCR on the cell surface.

Jurkat T cells were cultured as described above in Example 3. The cells were electroporated using the Neon Electroporation system (VOLTAGE=1350V, PULSE LENGTH=10, # OF PULSES=3) and transduced with one of the triple shmiR vectors indicated in Table 4 expressing multiple shmiRs against the different TCR subunits. The cells were a co-transduced with a K^{k} DNA construct which expresses truncated MHC class I molecule H-2K^{k} as a surface marker to select transfected cells. Untreated wild-type and mutant (lacking TCR complex) Jurkat T cells were used as controls as well as wild-type Jurkat T cells transduced with an unrelated triple shmiR construct targeting Hepatitis C.

After 20 h, the cells were sorted with MACSelect beads (Miltenyi) against K^{k} in order to select positively transduced cells and then the selected cells were cultured for 48 hours to allow recovery.

Cells were stained for flow cytometry using an antibody against TCR-α/β (eBioscience, Anti-Human alpha beta TCR FITC; cat. No. 11-9986) or a control antibody (eBioscience, Mouse IgG1 K Isotype Control FITC; cat. No 11-4714) in FACS buffer (10% FCS, 1×PBS). Cells were analyzed on a BD LSRII fluorescence-activated cell sorting machine (FACS).

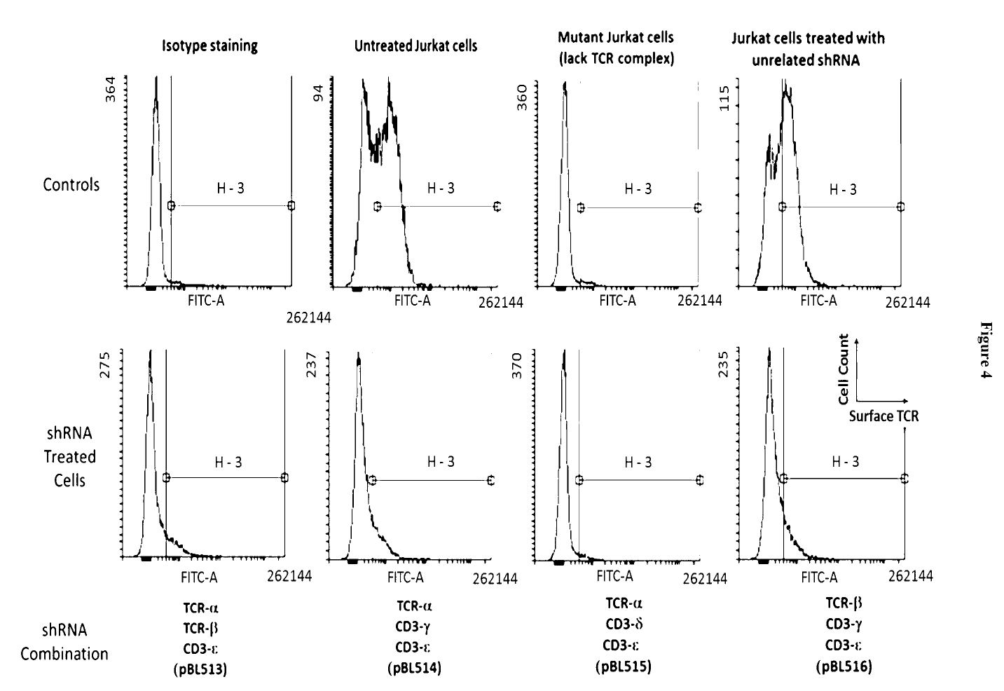

The resulting FACS plots are presented in FIG. 4. The analysis showed that the triple shmiR constructs were able to almost completely deplete the assembly of the TCR complex and prevent its display on the cell surface, with depletion rates of greater than 95%.

### Example 6—Inhibition of TCR-Mediated Signal Transduction in T Cells Activated with Anti-CD3 and Anti-CD28 Antibodies

This example demonstrates the ability of the triple shmiR constructs to prevent T cell activation mediated by TCR signal transduction in Jurkat T cells activated by anti-CD3 and anti-CD28 antibodies.

Jurkat T cells were cultured as described above in Example 3 and electroporated with the triple shmiR constructs described in Example 4 using the methods described in Example 5. After 20 h, the cells were then sorted with MACSelect beads (Miltenyi) against K^{k} for cells positively transduced. The selected cells were cultured for 48 hours to allow recovery.

The transduced cells were then subsequently treated with anti-CD3 (5 ug/mL solution of anti-CD3c, OKT3) and anti-CD28 antibodies (soluble anti-CD28 to cells at 2 ug/mL) for 48 hours in order to stimulate the activation of the T cells.

### ELISA

In order to measure TCR mediated signal transduction, the concentration of Interleukin-2 (IL-2) secreted by the activated T cells was measured by Enzyme-linked immunosorbent assay (ELISA). Following activation by anti-CD3 and anti CD28 antibodies as described above, the cells were incubated for 48 h and then the supernatant was harvested. TCR mediated T cell activation was then measured by ELISA against IL-2 in the supernatant of the activated cell culture. Untreated wild-type and mutant (lacking TCR) Jurkat T cells were provided as controls.

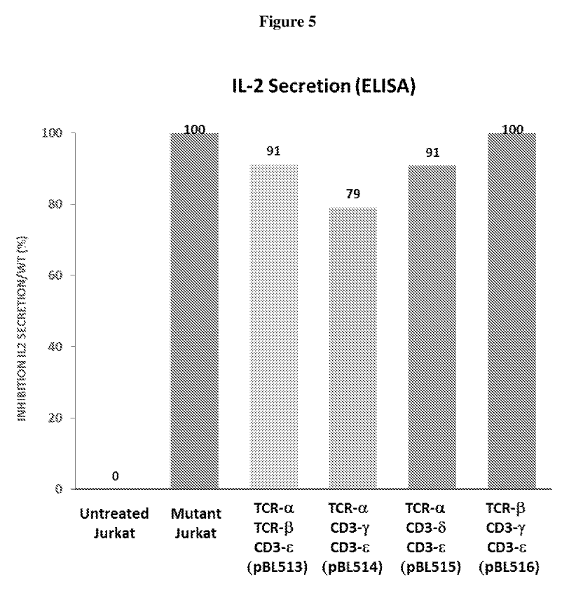

The results are presented in FIG. 5. All of the triple shmiR constructs tested inhibited TCR-mediated signal transduction, as measured by IL-2 secretion. The percentage inhibition ranged from 79% for pBL514 to 100% (IL-2 undetectable) for pBL516.

qPCR Analysis

To measure IL-2 mRNA levels, RNAs were harvested after 48 hours using Qiazol Reagent and RNA samples were quantified using a ND-1000 NanoDrop spectrophotometer (NanoDrop Technologies). cDNAs were generated by reverse transcription using ABI ‘High Capacity cDNA Reverse Transcription Kit’ Product No. 4368813 and Ambion ‘Superase Inhibitor”. cDNAs were used for quantitative PCR reaction using Taqman qPCR master mix in a total of 10 ul reaction volumes. The PCR reaction were carried out as follows: 2 minutes at 50° C., 10 minutes at 95° C. followed by 40 cycles: 15 seconds at 95° C., 1 minute at 60° C. Primers were designed using GenScript TaqMan primer design tool (https://www.genscript.com/ssl-bin/app/primer).

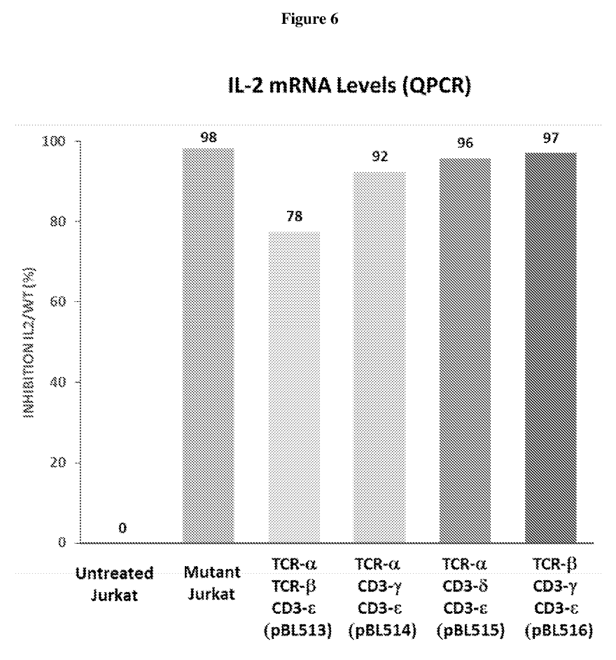

The expression levels of IL-2 mRNA were normalized to GAPDH. Expression levels were calculated according to the total copies as determine by a standard curve and converted to percent inhibition relative to the untreated wild-type Jurkat T cells control. The resulting percent inhibition of the endogenous expression of IL-2 in the Jurkat T cells by the triple shmiR constructs is presented in FIG. 6. The triple shmiR constructs knocked down the expression of IL-2 with percent inhibition ranging between 78% to 97%.

### Example 7—Inhibition of TCR-Mediated Signal Transduction in T Cells Activated Through Antigen Presenting Cell Co-Culture

This example demonstrates the ability of the triple shmiR constructs to prevent T cell activation mediated by TCR signal transduction in Jurkat T cells activated by antigen presenting cells.

Jurkat T cells were cultured as described above in Example 3 and electroporated with the triple shmiR constructs described in Example 4 using the methods described in Example 5. After 20 h, the cells were then sorted with MACSelect beads (Miltenyi) against K^{k} for cells positively transduced. The selected cells were cultured for 48 hours to allow recovery.

Transduced cells were subsequently co-cultured for 5 hours with Raji B cells (antigen presenting cells) loaded with Staphylococcal enterotoxins in order to activate the T cells through TCR-mediated signal transduction. Staphylococcal enterotoxins are exotoxins produced by Staphylococcus aureus that possess emetic and superantigenic properties which are defined by their unique ability to stimulate a large variety of T cells. Such superantigens stimulate the production of cytokines such as IL-2 via TCR signal transduction.

Therefore, in order to confirm the results observed in Example 6 and to measure T cell functionality upon TCR knock-down by the triple shmiR constructs, the concentration of IL-2 secreted by the Jurkat T cells was measured. Untreated wild-type and mutant (lacking TCR) Jurkat T cells were provided as controls, as well as untreated wild-type Jurkat T cells that were not co-cultured with Raji B Cells.

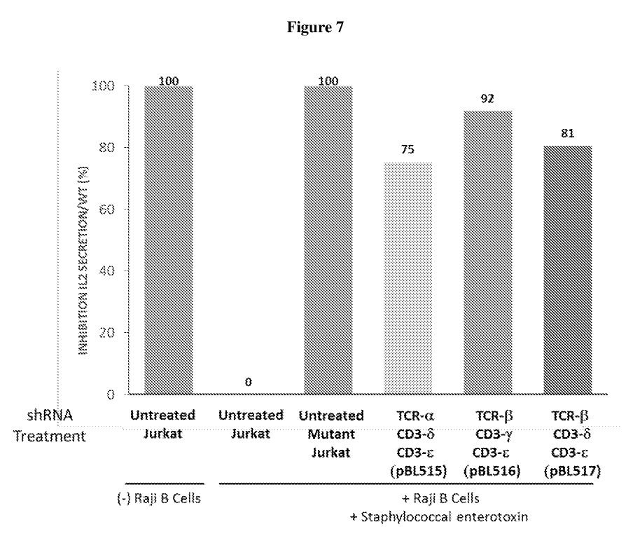

Following 5 hours of co-culturing the Jurkat T cells with the Raji B cells, the supernatant was harvested and T cell activation was measured by ELISA against IL-2. FIG. 7 shows the percentage inhibition of IL-2 secretion by Jurkat T cells transduced with the triple shmiR constructs. All of the triple shmiR constructs tested inhibited T cell activation, measued by IL-2 secretion, by up to 92%. Together with Example 6, these results confirmed that the triple shmiR constructs provided in Table 4 are able to inhibit TCR mediated signal transduction.

### Example 8—Triple shmiR Constructs do not Prevent TCR-Independent Activation

Given the strong inhibition of TCR-mediated activation by the triple shmiR constructs described in Example 6 and Example 7, it was assessed whether the transduced T cells were still able to be activated via a TCR-independent pathway.

Jurkat T cells were cultured as described above in Example 3 and electroporated with triple shmiR constructs as described in Example 4 using the method described in Example 5. After 20 h, the cells were then sorted with MACSelect beads (Miltenyi) against K^{k} for positively transduced. The selected cells were cultured for 48 hours to allow recovery.

In order to stimulate activation, the cells were treated with phorbol 12-myristate 13-acetate (PMA, SigmaAldrich #P8139, long/mL) and Ionomycin (SigmaAldrich #I0634, 1 ug/mL) for 4 hours in culture. Following activation, the supernatant was harvested and T cell activation was then measured by ELISA against IL-2. Untreated wild-type and mutant (lacking TCR) Jurkat T cells were provided as controls, as well as wild-type Jurkat T cells transduced with an unrelated shRNA targeting Hepatitis C viral proteins.

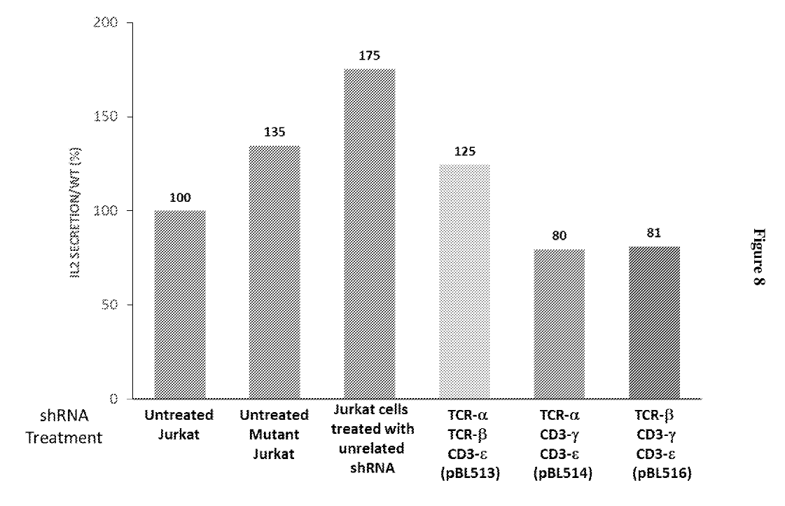

The concentration of IL2 secreted by the cells transduced with the triple shmiR constructs (relative to untreated cells) is shown in FIG. 8. These data show that the triple shmiR constructs did not significantly affect the TCR-independent T cell activation pathway. Cells transduced with the pBL513 construct displayed a 25% increase in IL-2 secretion relative to untreated cells. Whereas pBL514 and pBL516 treated cells maintained about 80% of the level IL-2 secreted by untreated cells.

### Example 9—Triple shmiR Constructs do not Disrupt Cell Cycle Distribution

This example demonstrates that the triple shmiR constructs do not have an adverse effect on the cycling of the transduced cells, as measured by FACS analysis.

Jurkat T cells were cultured as described above in Example 3 and electroporated with triple shmiR constructs described in Example 4 using the method described in Example 5. After 20 h, the cells were then sorted with MACSelect beads (Miltenyi) against K^{k} for positively transduced. The selected cells were cultured for 48 hours to allow recovery.

The cells were then pulsed with the thymidine analog bromodeoxyuridine (BrdU), which incorporates into newly synthesized DNA. The cells were incubated for 1 h and were then stained for flow cytometry with 7-aminoactinomycin D (7AAD), which binds total DNA. The cells were labelled with fluorescent antibodies against BrdU and 7AAD (BD Bioscience) in FACS buffer (10% FCS, 1×PBS) along with an anti-TCR antibody (eBioscience, TCR-PE; cat. No. 12-9986-42). Cells were gated on the TCR-negative populations and the cell cycle populations were then analysed according to a BrdU FITC assay. The analysis was performed on a BD LSRII FACS machine.

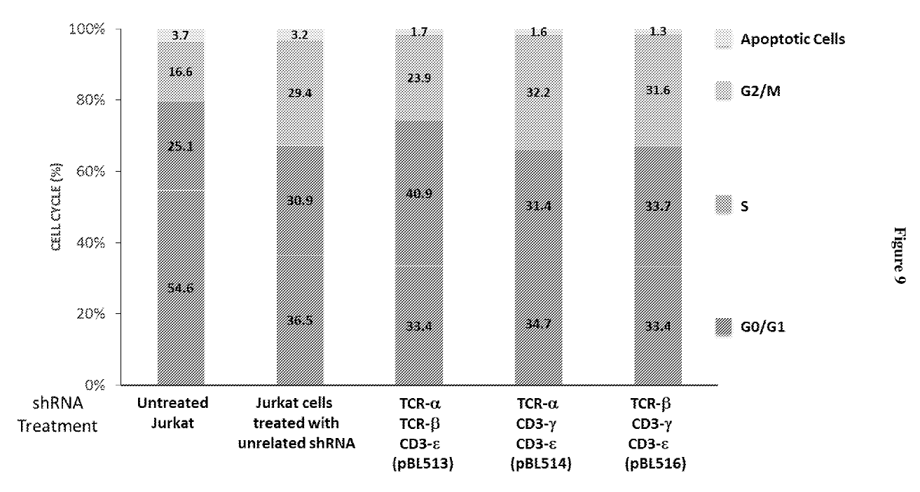

The bar graph in FIG. 9 demonstrates the percentage of TCR-less cells in each stage of the cell cycle, G0/G1, S, G2/M, as determined by the assay. There were no significant changes in the cell cycle distribution in the T cells lacking the TCR complex due to knockdown by the triple shmiR constructs compared to untreated cells.

### Example 10—Preparation of Clinical Constructs for the Simultaneous Knockdown of TCR and Replacement with a Chimeric Antigen Receptor

In order to direct the simultaneous gene silencing of endogenous TCR and replacement with a chimeric antigen receptor, lentiviral vectors expressing three of the selected shmiRs in combination with a chimeric antigen receptor (CAR) targeting CD19 are created. CARs are engineered receptors, which essentially enable the grafting of an arbitrary specificity onto an immune effector cell such as a T cell. CD19 is a B cell specific antigen and is the target of CARs for the treatment of B cell malignancies.

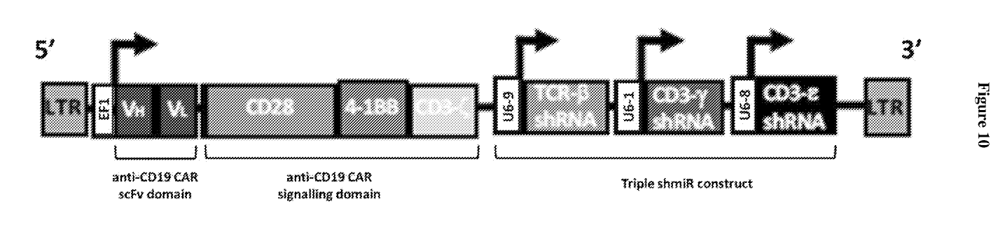

An example of a construct described above is presented in FIG. 10. The construct is generated by subcloning the sequence of a triple shmiR construct of Example 4 into a Lentiviral vector, either upstream or downstream of a sequence encoding a CAR. The exemplary construct depicted in FIG. 10 is comprised of a 5′LTR, followed by the EF1 promoter, the CD19 Single Chain Variable Fragment (scFv; Variable Heavy, VH; linker; Variable Light, VL), spacer domain, the signalling domain (that includes the CD28 transmembrane domain, 41BB and CD3c), and a transcriptional termination sequence, followed by the U6-9 promoter, a sequence coding for shmiR-TCR-β_2 (SEQ ID NO:159), U6-1 promoter, a sequence coding for shmiR-CD3-γ_2 (SEQ ID NO: 164), U6-8 promoter, a sequence coding for shmiR-CD3-ε_3 shmiR (SEQ ID NO: 171), a transcriptional termination sequence, and the 3′ LTR.

### Example 11—Expression Levels of shmiRs from Triple Hairpin Constructs

This example demonstrates the level of hairpin expression of each individual shmiR when expressed by triple hairpin construct in Jurkat T cells and unanticipated low level expression of CD3-ε-1 shmiR.

Jurkat T cells were cultured as described in Example 3 and electroporated with the triple shmiR constructs designated pBL513, pBL514, or pBL516 (described in Example 4 and Table 4). The selected cells were cultured for 48 hours to allow recovery.

Transduced cells were subsequently collected and RNA harvested using Qiazol Reagent and purified RNA samples resuspended in nuclease free water. RNA samples were quantified using a ND-1000 NanoDrop spectrophotometer (NanoDrop Technologies).

### Next Generation Sequencing (NGS)

100 ng of DNase treated total RNA at a concentration of 5 ng/ul were sent to SeqMatic (44846 Osgood Rd. Fremont, Calif. 94539) for Next Generation Sequencing (NGS).

### Quantimir RT Assay

cDNA was generated by reverse transcription using System Biociences (SBI) ‘QuantiMir RT Kit’ Cat. # RA420A-1. cDNA was used for quantitative PCR reaction using 2×SYBR PCR master mix in a total of 10 ul reaction volume with 10 uM universal reverse primer and 10 uM hairpin-specific primer. The PCR reaction was carried out as follows: 2 minutes at 50 C, 10 minutes at 95 C followed by 40 cycles: 15 seconds at 95 C, 1 minute at 60 C. Primers were designed using GenScript TaqMan primer design tool (https://www.genscript.com/ssi-bin/app/primer). The expression level of each hairpin was normalized to total cell number. Expression levels were calculated according to the total copies as determined by a standard curve.

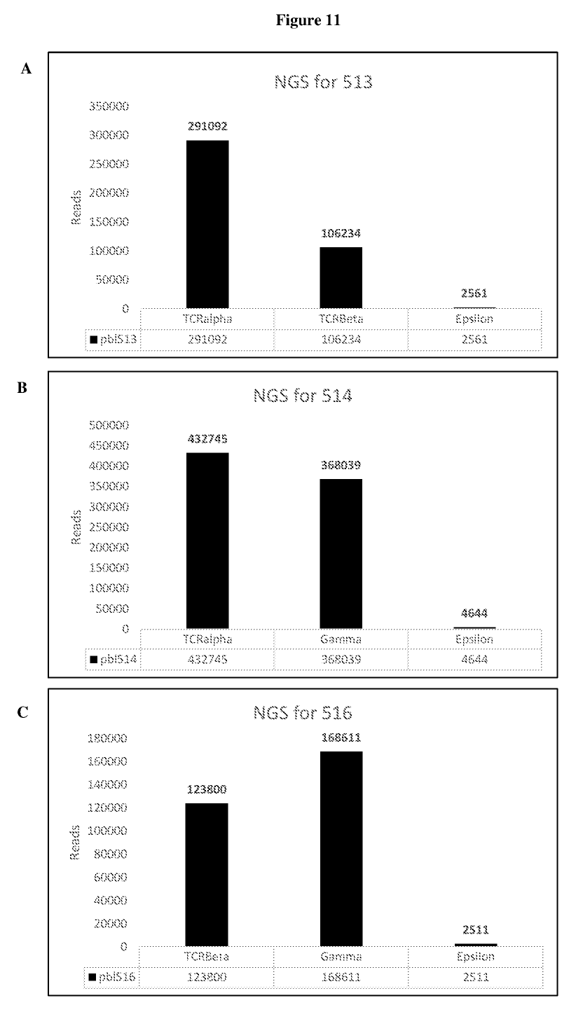

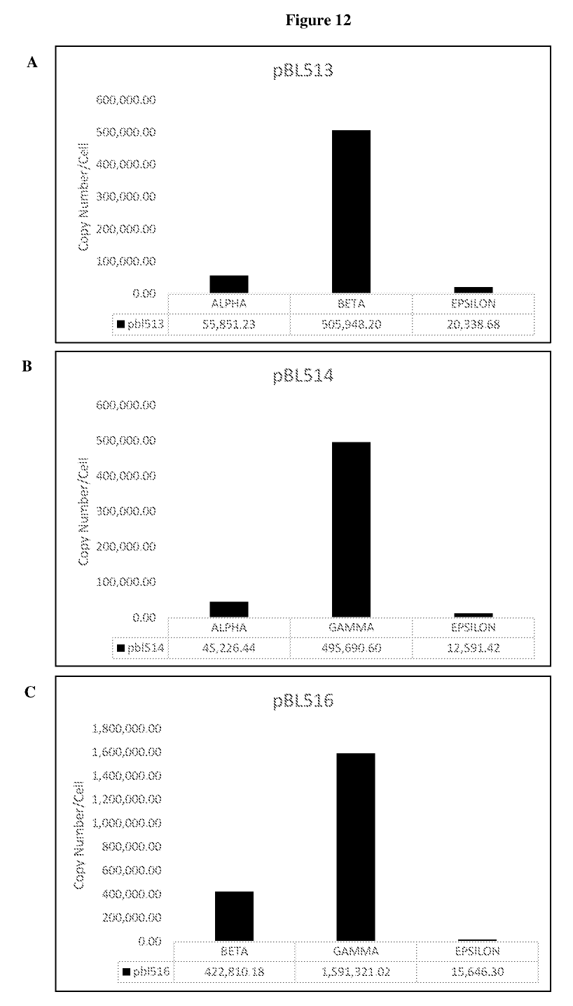

The expression levels of the shmiR-CD3-ε_3, as determined by both Quantimir assay and NGS, was significantly lower than the other two hairpins. As shown in FIGS. 11 and 12, low levels of hairpin expression were observed regardless of which shmiR was present in other positions of the construct.

### Example 12—Replacement of the Third Promoter in the Triple shmiR Constructs Concomitantly Expressing Three shmiRs Targeting TCR Subunits

To overcome low levels of expression of shmiR-CD3-ε-3 observed in pBL513, pBL514 and pBL516, the U6-8 promoter which drove expression of shmiR-CD3-ε_3 in the last position of these constructs, was replaced with an H1 promoter and cloned into a lentiviral vector (CD512B-1; SBI) to produce the constructs pBL528 (SEQ ID NO: 176), pBL529 (SEQ ID NO: 177) and pBL530 (SEQ ID NO: 178).

### Example 13—Enhanced Biological Activity of H1 Promoter-Modified Triple Hairpin Constructs

This example demonstrates the ability of the H1 promoter-modified triple shmiR constructs (pBL528, pBL529 and pBL529) to down regulate TCR components more efficiently than the corresponding triple constructs using the U6-8 promoter. This was shown using both dual luciferase assays and inhibition of IL-2 production in transfected

### Jurkat Cells.

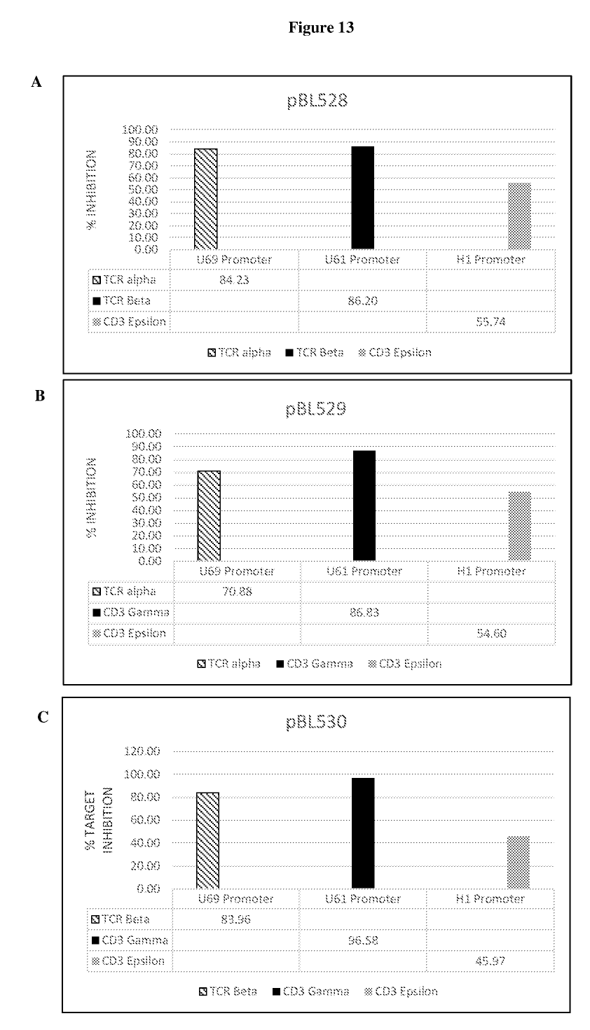

Jurkat T cells were cultured and transduced with plasmid DNAs pBL528, pBL529 or pBL 530 and selected as described above in Example 3. For dual luciferase assays, cells were also transduced with appropriate luciferase reporter constructs. As shown in FIG. 13 the H1 promoter modified constructs showed significantly increased activity with inhibition against a CD-3 reporter construct compared to the original constructs, consistent with increased expression of shmiR-CD3-ε_3.

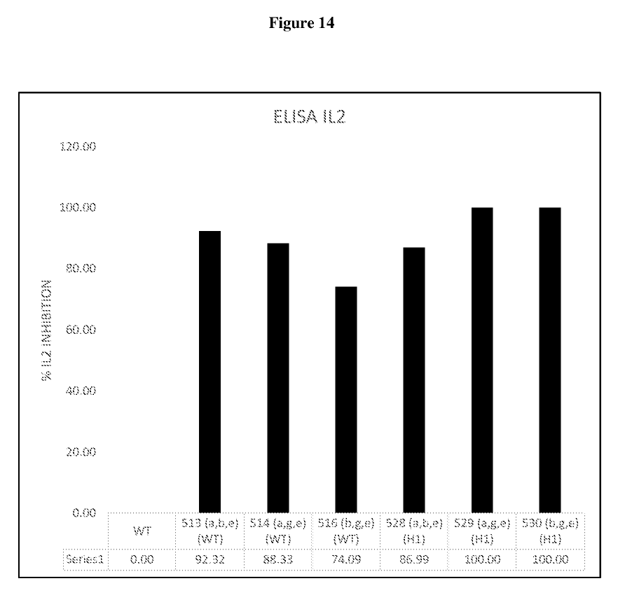

Enhanced biological activity for H1 containing constructs was confirmed using inhibition of IL-2 secretion in transduced cells as described in Example 6. Cells were transfected with pBL528, pBL529 or pBL 530, selected and stimulated with antibodies and IL-2 secretion assayed using ELISA assays as described in Example 6. As shown in FIG. 14, constructs using the H1 promoter in place of the U6-8 promoter showed greater inhibition of IL-2 secretion.

### Example 14—Preparation of Clinical Candidate

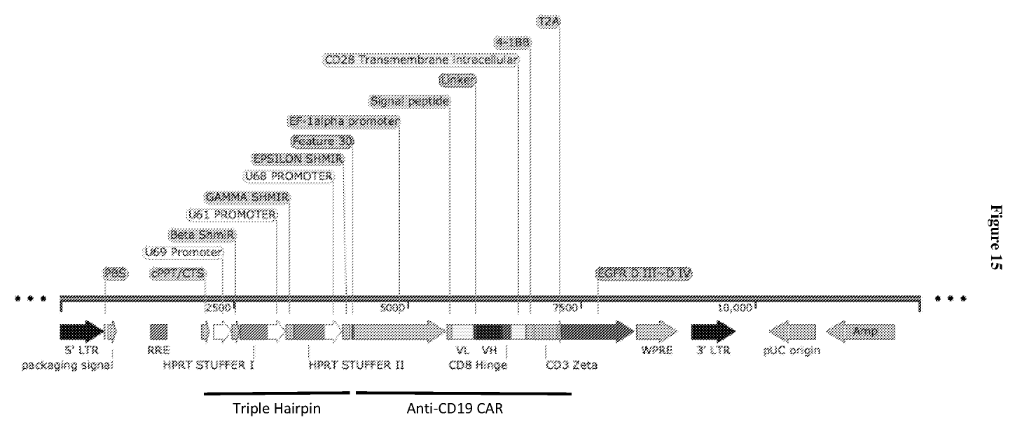

Based on the data outlined in Example 13, the triple shmiR insert from pBL530 was cloned into a lentiviral vector containing a CAR construct (Creative Biolabs) to generate pBL531 (SEQ ID NO: 179). pBL531 comprises: a 5′ lentiviral terminal repeat (LTR) sequence; the polymerase-III promoter U6-9 positioned upstream of shmiR-TCR-β_5; an HPRT derived stuffer sequence; a U6-1 promoter upstream of shmiR CD3-γ_2; a second HPRT Stuffer; and the H1 promoter upstream of shmiR-CD3-ε_3; followed by the anti-CD19 CAR (EF1 promoter, the CD19 Single Chain Variable Fragment (scFv; Variable Heavy, VH; linker; Variable Light, VL), spacer domain, the signaling domain (that includes the CD28 transmembrane domain, 41BB and CD3 zeta), and a transcriptional termination sequence); followed by a 3′ LTR sequence. A map of pBL531 is shown in FIG. 15.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A DNA-directed RNA interference (ddRNAi) construct comprising two or more nucleic acids with a DNA sequence coding for a short hairpin micro-RNA (shmiR), wherein each shmiR comprises:
an effector sequence of at least 17 nucleotides in length;
an effector complement sequence;
a stemloop sequence; and
a primary micro RNA (pri-miRNA) backbone;
wherein the effector sequence of each shmiR is substantially complementary to a region of corresponding length in a mRNA transcript for a T-cell receptor (TCR) complex subunit selected from the group consisting of: CD3-ε, TCR-α, TCR-β, CD3-γ and CD3-δ.

2. The ddRNAi construct of claim 1, wherein each shmiR comprises, in a 5′ to 3′ direction:
a 5′ flanking sequence of the pri-miRNA backbone;
the effector complement sequence;
the stemloop sequence;
the effector sequence; and
a 3′ flanking sequence of the pri-miRNA backbone.

3. The ddRNAi construct of claim 2, wherein:
(i) the stemloop sequence is the sequence set forth in SEQ ID NO: 97; and/or
(ii) the pri-miRNA backbone is a pri-miR-31a backbone; and/or
(iii) the 5′ flanking sequence of the pri-miRNA backbone is set forth in SEQ ID NO: 98 and the 3′ flanking sequence of the pri-miRNA backbone is set forth in SEQ ID NO: 99.

4. (canceled)

5. (canceled)

6. The ddRNAi construct according to claim 1, wherein the two or more nucleic acids are selected from:
(a) (i) a nucleic acid comprising or consisting of a DNA sequence coding for shmiR-CD3-ε which comprises an effector sequence which is substantially complementary to a region of corresponding length in a mRNA transcript for the CD3-ε subunit;
(ii) a nucleic acid comprising or consisting of a DNA sequence coding for shmiR-CD3-γ which comprises an effector sequence which is substantially complementary to a region of corresponding length in a mRNA transcript for the CD3-γ subunit; and
(iii) a nucleic acid comprising or consisting of a DNA sequence coding for shmiR-TCR-β which comprises an effector sequence which is substantially complementary to a region of corresponding length in a mRNA transcript for the TCR-β subunit;
(b) (i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-TCR-β which comprises an effector sequence which is substantially complementary to a region of corresponding length in a mRNA transcript for the TCR-β subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit; or
(c) (i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-CD3-γ which comprises an effector sequence which is substantially complementary to a region of corresponding length in a mRNA transcript for the CD3-γ subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit;
(d) (i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-CD3-δ which comprises an effector sequence which is substantially complementary to a region of corresponding length in a mRNA transcript for the CD3-δ subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

7. The ddRNAi construct of claim 6, comprising:
(a) (i) a nucleic acid comprising or consisting of a DNA sequence coding for shmiR-CD3-ε which comprises an effector sequence which is substantially complementary to a region of corresponding length in a mRNA transcript for the CD3-ε subunit;
(ii) a nucleic acid comprising or consisting of a DNA sequence coding for shmiR-CD3-γ which comprises an effector sequence which is substantially complementary to a region of corresponding length in a mRNA transcript for the CD3-γ subunit; and
(iii) a nucleic acid comprising or consisting of a DNA sequence coding for shmiR-TCR-β which comprises an effector sequence which is substantially complementary to a region of corresponding length in a mRNA transcript for the TCR-β subunit;
(b) (i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-TCR-β which comprises an effector sequence which is substantially complementary to a region of corresponding length in a mRNA transcript for the TCR-β subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit;
(c) (i) a nucleic acid comprising or consisting of a DNA sequence coding for shmiR-TCR-α comprising an effector sequence which is substantially complementary to a region of corresponding length in a mRNA transcript for the TCR-α subunit;
(ii) a nucleic acid comprising or consisting of a DNA sequence coding for shmiR-CD3-γ comprising an effector sequence which is substantially complementary to a region of corresponding length in a mRNA transcript for the CD3-γ subunit; and
(iii) a nucleic acid comprising or consisting of a DNA sequence coding for shmiR-CD3-ε comprising an effector sequence which is substantially complementary to a region of corresponding length in a mRNA transcript for the CD3-ε subunit; or
(d) (i) a nucleic acid comprising or consisting of a DNA sequence coding for shmiR-TCR-α which comprises an effector sequence which is substantially complementary to a region of corresponding length in a mRNA transcript for the TCR-α subunit;
(ii) a nucleic acid comprising or consisting of a DNA sequence coding for shmiR-CD3-δ which comprises an effector sequence which is substantially complementary to a region of corresponding length in a mRNA transcript for the CD3-δ subunit; and
(iii) a nucleic acid comprising or consisting of a DNA sequence coding for shmiR-CD3-ε which comprises an effector sequence which is substantially complementary to a region of corresponding length in a mRNA transcript for the CD3-ε subunit.

8. The ddRNAi construct of claim 6, wherein:
(a) (i) shmiR-CD3-ε comprises an effector sequence set forth in SEQ ID NO: 134;
(ii) shmiR-CD3-γ comprises an effector sequence set forth in SEQ ID NO: 120; and
(iii) shmiR-TCR-β comprises an effector sequence set forth in SEQ ID NO: 116;
(b) (i) shmiR-TCR-α comprises an effector sequence set forth in SEQ ID NO: 100;
(ii) shmiR-TCR-β comprises an effector sequence set forth in SEQ ID NO: 116; and
(iii) shmiR-CD3-ε comprises an effector sequence set forth in SEQ ID NO: 134;
(c) (i) shmiR-TCR-α comprises an effector sequence set forth in SEQ ID NO: 100;
(ii) shmiR-CD3-γ comprises an effector sequence set forth in SEQ ID NO: 120; and
(iii) shmiR-CD3-ε comprises an effector sequence set forth in SEQ ID NO: 134; or
(d) (i) shmiR-TCR-α comprises an effector sequence set forth in SEQ ID NO: 100;
(ii) shmiR-CD3-δ comprises an effector sequence set forth in SEQ ID NO: 126; and
(iii) shmiR-CD3-ε comprises an effector sequence set forth in SEQ ID NO: 134.

9. The ddRNAi construct according to claim 6, wherein:
(a) (i) shmiR-CD3-ε comprises an effector sequence set forth in SEQ ID NO: 134 and an effector complement sequence set forth in SEQ ID NO: 135;
(ii) shmiR-CD3-γ comprises an effector sequence set forth in SEQ ID NO: 120 and an effector complement sequence set forth in SEQ ID NO: 121; and
(iii) shmiR-TCR-β comprises an effector sequence set forth in SEQ ID NO: 116 and an effector complement sequence set forth in SEQ ID NO: 117;
(b) (i) shmiR-TCR-α comprises an effector sequence set forth in SEQ ID NO: 100 and an effector complement sequence set forth in SEQ ID NO: 101;
(ii) shmiR-TCR-β comprises an effector sequence set forth in SEQ ID NO: 116 and an effector complement sequence set forth in SEQ ID NO: 117; and
(iii) shmiR-CD3-ε comprises an effector sequence set forth in SEQ ID NO: 134 and an effector complement sequence set forth in SEQ ID NO: 135;
(c) (i) shmiR-TCR-α comprises an effector sequence set forth in SEQ ID NO: 100 and an effector complement sequence set forth in SEQ ID NO: 101;
(ii) shmiR-CD3-γ comprises an effector sequence set forth in SEQ ID NO: 120 and an effector complement sequence set forth in SEQ ID NO: 121; and
(iii) shmiR-CD3-ε comprises an effector sequence set forth in SEQ ID NO: 134 and an effector complement sequence set forth in SEQ ID NO: 135; or
(d) (i) shmiR-TCR-α comprises an effector sequence set forth in SEQ ID NO: 100 and an effector complement sequence set forth in SEQ ID NO: 101;
(ii) shmiR-CD3-δ comprises an effector sequence set forth in SEQ ID NO: 126 and an effector complement sequence set forth in SEQ ID NO: 127; and
(iii) shmiR-CD3-ε comprises an effector sequence set forth in SEQ ID NO: 134 and an effector complement sequence set forth in SEQ ID NO: 135.

10. The ddRNAi construct according to claim 6, wherein:
(a) (i) shmiR-CD3-ε comprises or consists of a sequence set forth in SEQ ID NO: 153;
(ii) shmiR-CD3-γ comprises or consists of a sequence set forth in SEQ ID NO: 146; and
(iii) shmiR-TCR-β comprises or consists of a sequence set forth in SEQ ID NO: 144;
(b) (i) shmiR-TCR-α comprises or consists of a sequence set forth in SEQ ID NO: 136;
(ii) shmiR-TCR-β comprises or consists of a sequence set forth in SEQ ID NO: 144; and
(iii) shmiR-CD3-ε comprises or consists of a sequence set forth in SEQ ID NO:153;
(c) (i) shmiR-TCR-α comprises or consists of a sequence set forth in SEQ ID NO: 136;
(ii) shmiR-CD3-γ comprises or consists of a sequence set forth in SEQ ID NO: 146; and
(iii) shmiR-CD3-ε comprises or consists of a sequence set forth in SEQ ID NO: 153; or
(d) (i) shmiR-TCR-α comprises or consists of a sequence set forth in SEQ ID NO: 136;
(ii) shmiR-CD3-δ comprises or consists of a sequence set forth in SEQ ID NO: 149; and
(iii) shmiR-CD3-ε comprises or consists of a sequence set forth in SEQ ID NO: 153.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The ddRNAi construct according to claim 1, comprising a RNA pol III promoter upstream of each nucleic acid coding for a shmiR, optionally wherein each RNA pol III promoter is a U6 selected from the group consisting of a U6-9 promoter, a U6-1 promoter and a U6-8 promoter, or a H1 promoter.

27. (canceled)

28. (canceled)

29. A DNA construct comprising:
(a) a ddRNAi construct according to claim 1; and
(b) a chimeric antigen receptor (CAR) construct comprising nucleic acid with a DNA sequence coding for a CAR.

30. The DNA construct according to claim 29, wherein the CAR comprises an antigen binding domain.

31. The DNA construct according to claim 30, wherein:
(i) the antigen binding domain is a binding protein, optionally wherein the binding protein is an antibody or an antigen binding domain thereof; and/or
(ii) the antigen binding domain binds specifically to a tumor antigen; or
(iii) the antigen binding domain binds specifically to a virus antigen or viral-induced antigen found on the surface of an infected cell.

32. (canceled)

33. (canceled)

34. (canceled)

35. The DNA construct according to claim 29, wherein:
(i) the DNA sequence coding for the CAR is operably-linked to a promoter comprised within the CAR construct and positioned upstream of the DNA sequence coding the CAR; and/or
(ii) the DNA construct comprises, in a 5′ to 3′ direction, the ddRNAi construct and the CAR construct; or
(iii) the DNA construct comprises, in a 5′ to 3′ direction, the CAR construct and the ddRNAi construct.

36. (canceled)

37. (canceled)

38. An expression vector comprising a ddRNAi construct according to claim 1 or a DNA construct comprising said ddRNAi construct and a DNA sequence encoding for a chimeric antigen receptor (CAR).

39. The expression vector according to claim 38, wherein the expression vector is a plasmid or minicircle, or a viral vector selected from the group consisting of an adeno-associated viral (AAV) vector, a retroviral vector, an adenoviral (AdV) vector and a lentiviral (LV) vector.

40. (canceled)

41. A T-cell comprising a ddRNAi construct according to claim 1, a DNA construct comprising said ddRNAi construct and a DNA sequence encoding for a chimeric antigen receptor (CAR), or an expression vector comprising said ddRNAi construct or DNA construct.

42. The T-cell according to claim 41, wherein:
(i) the T-cell does not express a functional TCR;
(ii) the T cell exhibits reduced cell-surface expression of at least two component of the TCR complex; and/or
(iii) the T cell expresses a chimeric antigen receptor (CAR).

43. (canceled)

44. (canceled)

45. The T-cell according to claim 42, wherein the CAR comprises an antigen binding domain.

46. The T-cell according to claim 45, wherein:
(i) the antigen binding domain is a binding protein, optionally wherein the binding protein is an antibody or an antigen binding domain thereof; and/or
(ii) the antigen binding domain binds specifically to a tumor antigen; or
(iii) the antigen binding domain binds specifically to a virus antigen or viral-induced antigen found on the surface of an infected cell.

47. (canceled)

48. (canceled)

49. (canceled)

50. A composition comprising:
(i) a ddRNAi construct according to claim 1, a DNA construct comprising said ddRNAi construct and a chimeric antigen receptor (CAR), an expression vector comprising said ddRNAi construct or DNA construct, or a T-cell comprising comprising said ddRNAi construct, DNA construct or expression vector;
(ii) one or more pharmaceutically acceptable carriers or diluents.

51. (canceled)

52. A method of producing a T-cell which does not express a functional TCR, said method comprising introducing into a T-cell one or more of a ddRNAi construct according to claim 1, a DNA construct comprising said ddRNAi construct and a chimeric antigen receptor (CAR), an expression vector comprising said ddRNAi construct or DNA construct, or a composition comprising said ddRNAi construct, DNA construct or expression vector.

53. A method of producing a T-cell which does not express a functional TCR but which expresses a chimeric antigen receptor (CAR), said method comprising:
(i) introducing into a T-cell one or more of a DNA construct of claim 29, an expression vector comprising said DNA construct or a composition comprising said DNA construct or expression vector comprising same; and
(ii) optionally HLA typing the T-cell which is produced at (i).

54. (canceled)

55. (canceled)

56. (canceled)

57. A method of preventing or treating cancer, graft versus host disease, infection, one or more autoimmune disorders, transplantation rejection, or radiation sickness in an individual in need thereof, comprising administering to said individual a T-cell of claim 41 or a composition comprising same.

58. The method according to claim 57, wherein the T-cell which is administered to the individual is an allogeneic T-cell or a non-autologous T-cell.

59. (canceled)

60. A cell bank comprising a plurality of T-cells of different HLA types which do not express a functional TCR, wherein the cell bank comprises at least one T-cell according to claim 41.


SEQUENCE LISTING (194 sequences)

SEQ ID NO: 1 - RNA, 21 nt, Artificial sequence
  RNA effector sequence for shRNA designated TCR- alpha_1
ucuguuucaaagcuuuucucg

SEQ ID NO: 2 - RNA, 21 nt, Artificial sequence
  RNA effector complement sequence for shRNA designated TCR-alpha_1
cgagaaaagcuuugaaacaga

SEQ ID NO: 3 - RNA, 21 nt, Artificial sequence
  RNA effector sequence for shRNA designated TCR- alpha_2
ucguaucuguuucaaagcuuu

SEQ ID NO: 4 - RNA, 21 nt, Artificial sequence
  RNA effector complement sequence for shRNA designated TCR-alpha_2
aaagcuuugaaacagauacga

SEQ ID NO: 5 - RNA, 21 nt, Artificial sequence
  RNA effector sequence for shRNA designated TCR- alpha_3
uagguucguaucuguuucaaa

SEQ ID NO: 6 - RNA, 21 nt, Artificial sequence
  RNA effector complement sequence for shRNA designated TCR-alpha_3
uuugaaacagauacgaaccua

SEQ ID NO: 7 - RNA, 21 nt, Artificial sequence
  RNA effector sequence for shRNA designated TCR- alpha_4
aaguuuagguucguaucuguu

SEQ ID NO: 8 - RNA, 21 nt, Artificial sequence
  RNA effector complement sequence for shRNA designated TCR-alpha_4
aacagauacgaaccuaaacuu

SEQ ID NO: 9 - RNA, 21 nt, Artificial sequence
  RNA effector sequence for shRNA designated TCR- alpha_5
uuugaaaguuuagguucguau

SEQ ID NO: 10 - RNA, 21 nt, Artificial sequence
  RNA effector complement sequence for shRNA designated TCR-alpha_5
auacgaaccuaaacuuucaaa

SEQ ID NO: 11 - RNA, 21 nt, Artificial sequence
  RNA effector sequence for shRNA designated TCR- alpha_6
agguuuugaaaguuuagguuc

SEQ ID NO: 12 - RNA, 21 nt, Artificial sequence
  RNA effector complement sequence for shRNA designated TCR-alpha_6
gaaccuaaacuuucaaaaccu

SEQ ID NO: 13 - RNA, 21 nt, Artificial sequence
  RNA effector sequence for shRNA designated TCR- beta_1
accagcucagcuccacguggu

SEQ ID NO: 14 - RNA, 21 nt, Artificial sequence
  RNA effector complement sequence for shRNA designated TCR- beta_1
accacguggagcugagcuggu

SEQ ID NO: 15 - RNA, 21 nt, Artificial sequence
  RNA effector sequence for shRNA designated TCR- beta_2
ccauucacccaccagcucagc

SEQ ID NO: 16 - RNA, 21 nt, Artificial sequence
  RNA effector complement sequence for shRNA designated TCR- beta_2
gcugagcuggugggugaaugg

SEQ ID NO: 17 - RNA, 21 nt, Artificial sequence
  RNA effector sequence for shRNA designated TCR- beta_3
gaccccacugugcaccuccuu

SEQ ID NO: 18 - RNA, 21 nt, Artificial sequence
  RNA effector complement sequence for shRNA designated TCR- beta_3
aaggaggugcacagugggguc

SEQ ID NO: 19 - RNA, 21 nt, Artificial sequence
  RNA effector sequence for shRNA designated TCR- beta_4
gugcugaccccacugugcacc

SEQ ID NO: 20 - RNA, 21 nt, Artificial sequence
  RNA effector complement sequence for shRNA designated TCR- beta_4
ggugcacaguggggucagcac

SEQ ID NO: 21 - RNA, 21 nt, Artificial sequence
  RNA effector sequence for shRNA designated TCR- beta_5
caggcggcugcucaggcagua

SEQ ID NO: 22 - RNA, 21 nt, Artificial sequence
  RNA effector complement sequence for shRNA designated TCR- beta_5
uacugccugagcagccgccug

SEQ ID NO: 23 - RNA, 21 nt, Artificial sequence
  RNA effector sequence for shRNA designated TCR- beta_6
gagacccucaggcggcugcuc

SEQ ID NO: 24 - RNA, 21 nt, Artificial sequence
  RNA effector complement sequence for shRNA designated TCR- beta_6
gagcagccgccugagggucuc

SEQ ID NO: 25 - RNA, 21 nt, Artificial sequence
  RNA effector sequence for shRNA designated TCR- beta_7
gacuugacagcggaagugguu

SEQ ID NO: 26 - RNA, 21 nt, Artificial sequence
  RNA effector complement sequence for shRNA designated TCR- beta_7
aaccacuuccgcugucaaguc

SEQ ID NO: 27 - RNA, 21 nt, Artificial sequence
  RNA effector sequence for shRNA designated TCR- beta_8
ucaggcggcugcucaggcagu

SEQ ID NO: 28 - RNA, 21 nt, Artificial sequence
  RNA effector complement sequence for shRNA designated TCR- beta_8
acugccugagcagccgccuga

SEQ ID NO: 29 - RNA, 21 nt, Artificial sequence
  RNA effector sequence for shRNA designated TCR- beta_9
ucacccaccagcucagcucca

SEQ ID NO: 30 - RNA, 21 nt, Artificial sequence
  RNA effector complement sequence for shRNA designated TCR- beta_9
uggagcugagcugguggguga

SEQ ID NO: 31 - RNA, 21 nt, Artificial sequence
  RNA effector sequence for shRNA designated CD3-epsilon_1
ccuuucuauucuugcuccagu

SEQ ID NO: 32 - RNA, 21 nt, Artificial sequence
  RNA effector complement sequence for shRNA designated CD3-epsilon_1
acuggagcaagaauagaaagg

SEQ ID NO: 33 - RNA, 21 nt, Artificial sequence
  RNA effector sequence for shRNA designated CD3-epsilon_2
cacaggcuuggccuuggccuu

SEQ ID NO: 34 - RNA, 21 nt, Artificial sequence
  RNA effector complement sequence for shRNA designated CD3-epsilon_2
aaggccaaggccaagccugug

SEQ ID NO: 35 - RNA, 21 nt, Artificial sequence
  RNA effector sequence for shRNA designated CD3-epsilon_3
ggguugggaacaggugguggc

SEQ ID NO: 36 - RNA, 21 nt, Artificial sequence
  RNA effector complement sequence for shRNA designated CD3-epsilon_3
gccaccaccuguucccaaccc

SEQ ID NO: 37 - RNA, 21 nt, Artificial sequence
  RNA effector sequence for shRNA designated CD3-epsilon_4
cucauagucuggguugggaac

SEQ ID NO: 38 - RNA, 21 nt, Artificial sequence
  RNA effector complement sequence for shRNA designated CD3-epsilon_4
guucccaacccagacuaugag

SEQ ID NO: 39 - RNA, 21 nt, Artificial sequence
  RNA effector sequence for shRNA designated CD3-epsilon_5
ggaugggcucauagucugggu

SEQ ID NO: 40 - RNA, 21 nt, Artificial sequence
  RNA effector complement sequence for shRNA designated CD3-epsilon_5
acccagacuaugagcccaucc

SEQ ID NO: 41 - RNA, 21 nt, Artificial sequence
  RNA effector sequence for shRNA designated CD3-epsilon_6
agaauacaggucccgcuggcc

SEQ ID NO: 42 - RNA, 21 nt, Artificial sequence
  RNA effector complement sequence for shRNA designated CD3-epsilon_6
ggccagcgggaccuguauucu

SEQ ID NO: 43 - RNA, 21 nt, Artificial sequence
  RNA effector sequence for shRNA designated CD3-epsilon_7
cucugauucaggccagaauac

SEQ ID NO: 44 - RNA, 21 nt, Artificial sequence
  RNA effector complement sequence for shRNA designated CD3-epsilon_7
guauucuggccugaaucagag

SEQ ID NO: 45 - RNA, 21 nt, Artificial sequence
  RNA effector sequence for shRNA designated CD3-epsilon_8
uagucuggguugggaacaggu

SEQ ID NO: 46 - RNA, 21 nt, Artificial sequence
  RNA effector complement sequence for shRNA designated CD3-epsilon_8
accuguucccaacccagacua

SEQ ID NO: 47 - RNA, 21 nt, Artificial sequence
  RNA effector sequence for shRNA designated CD3-epsilon_9
uuccggaugggcucauagucu

SEQ ID NO: 48 - RNA, 21 nt, Artificial sequence
  RNA effector complement sequence for shRNA designated CD3-epsilon_9
agacuaugagcccauccggaa

SEQ ID NO: 49 - RNA, 21 nt, Artificial sequence
  RNA effector sequence for shRNA designated CD3-epsilon_10
ucaggccagaauacagguccc

SEQ ID NO: 50 - RNA, 21 nt, Artificial sequence
  RNA effector complement sequence for shRNA designated CD3-epsilon_10
gggaccuguauucuggccuga

SEQ ID NO: 51 - RNA, 21 nt, Artificial sequence
  RNA effector sequence for shRNA designated CD3-epsilon_11
auucaggccagaauacagguc

SEQ ID NO: 52 - RNA, 21 nt, Artificial sequence
  RNA effector complement sequence for shRNA designated CD3-epsilon_11
gaccuguauucuggccugaau

SEQ ID NO: 53 - RNA, 21 nt, Artificial sequence
  RNA effector sequence for shRNA designated CD3-epsilon_12
gauucaggccagaauacaggu

SEQ ID NO: 54 - RNA, 21 nt, Artificial sequence
  RNA effector complement sequence for shRNA designated CD3-epsilon_12
accuguauucuggccugaauc

SEQ ID NO: 55 - RNA, 21 nt, Artificial sequence
  RNA effector sequence for shRNA designated CD3-epsilon_13
uucuucauuaccaucuugccc

SEQ ID NO: 56 - RNA, 21 nt, Artificial sequence
  RNA effector complement sequence for shRNA designated CD3-epsilon_13
gggcaagaugguaaugaagaa

SEQ ID NO: 57 - RNA, 21 nt, Artificial sequence
  RNA effector sequence for shRNA designated CD3-delta_1
guaucuugaaggggcucacuu

SEQ ID NO: 58 - RNA, 21 nt, Artificial sequence
  RNA effector complement sequence for shRNA designated CD3-delta_1
aagugagccccuucaagauac

SEQ ID NO: 59 - RNA, 21 nt, Artificial sequence
  RNA effector sequence for shRNA designated CD3-delta_2
auauauuccucguggguccag

SEQ ID NO: 60 - RNA, 21 nt, Artificial sequence
  RNA effector complement sequence for shRNA designated CD3-delta_2
cuggacccacgaggaauauau

SEQ ID NO: 61 - RNA, 21 nt, Artificial sequence
  RNA effector sequence for shRNA designated CD3-delta_3
ucccaaagcaaggagcagagu

SEQ ID NO: 62 - RNA, 21 nt, Artificial sequence
  RNA effector complement sequence for shRNA designated CD3-delta_3
acucugcuccuugcuuuggga

SEQ ID NO: 63 - RNA, 21 nt, Artificial sequence
  RNA effector sequence for shRNA designated CD3-delta_4
aaagcagaagacucccaaagc

SEQ ID NO: 64 - RNA, 21 nt, Artificial sequence
  RNA effector complement sequence for shRNA designated CD3-delta_4
gcuuugggagucuucugcuuu

SEQ ID NO: 65 - RNA, 21 nt, Artificial sequence
  RNA effector sequence for shRNA designated CD3-delta_5
gucucauguccagcaaagcag

SEQ ID NO: 66 - RNA, 21 nt, Artificial sequence
  RNA effector complement sequence for shRNA designated CD3-delta_5
cugcuuugcuggacaugagac

SEQ ID NO: 67 - RNA, 21 nt, Artificial sequence
  RNA effector sequence for shRNA designated CD3-delta_6
auuccucguggguccaggaug

SEQ ID NO: 68 - RNA, 21 nt, Artificial sequence
  RNA effector complement sequence for shRNA designated CD3-delta_6
cauccuggacccacgaggaau

SEQ ID NO: 69 - RNA, 21 nt, Artificial sequence
  RNA effector sequence for shRNA designated CD3-delta_7
ccuauauauuccucguggguc

SEQ ID NO: 70 - RNA, 21 nt, Artificial sequence
  RNA effector complement sequence for shRNA designated CD3-delta_7
gacccacgaggaauauauagg

SEQ ID NO: 71 - RNA, 21 nt, Artificial sequence
  RNA effector sequence for shRNA designated CD3-delta_8
caccuauauauuccucguggg

SEQ ID NO: 72 - RNA, 21 nt, Artificial sequence
  RNA effector complement sequence for shRNA designated CD3-delta_8
cccacgaggaauauauaggug

SEQ ID NO: 73 - RNA, 21 nt, Artificial sequence
  RNA effector sequence for shRNA designated CD3-delta_9
caaggagcagaguggcaauga

SEQ ID NO: 74 - RNA, 21 nt, Artificial sequence
  RNA effector complement sequence for shRNA designated CD3-delta_9
ucauugccacucugcuccuug

SEQ ID NO: 75 - RNA, 21 nt, Artificial sequence
  RNA effector sequence for shRNA designated CD3-delta_10
cugagcaucaucucgaucucg

SEQ ID NO: 76 - RNA, 21 nt, Artificial sequence
  RNA effector complement sequence for shRNA designated CD3-delta_10
cgagaucgagaugaugcucag

SEQ ID NO: 77 - RNA, 21 nt, Artificial sequence
  RNA effector sequence for shRNA designated CD3-delta_11
auaucugucccauuacaccua

SEQ ID NO: 78 - RNA, 21 nt, Artificial sequence
  RNA effector complement sequence for shRNA designated CD3-delta_11
uagguguaaugggacagauau

SEQ ID NO: 79 - RNA, 21 nt, Artificial sequence
  RNA effector sequence for shRNA designated CD3-delta_12
uauaucugucccauuacaccu

SEQ ID NO: 80 - RNA, 21 nt, Artificial sequence
  RNA effector complement sequence for shRNA designated CD3-delta_12
agguguaaugggacagauaua

SEQ ID NO: 81 - RNA, 21 nt, Artificial sequence
  RNA effector sequence for shRNA designated CD3-delta_13
aaugacaucagugacaaugau

SEQ ID NO: 82 - RNA, 21 nt, Artificial sequence
  RNA effector complement sequence for shRNA designated CD3-delta_13
aucauugucacugaugucauu

SEQ ID NO: 83 - RNA, 21 nt, Artificial sequence
  RNA effector sequence for shRNA designated CD3-gamma_1
caaguguauuacagaaugugu

SEQ ID NO: 84 - RNA, 21 nt, Artificial sequence
  RNA effector complement sequence for shRNA designated CD3-gamma_1
acacauucuguaauacacuug

SEQ ID NO: 85 - RNA, 21 nt, Artificial sequence
  RNA effector sequence for shRNA designated CD3-gamma_2
ggacaggauggaguucgccag

SEQ ID NO: 86 - RNA, 21 nt, Artificial sequence
  RNA effector complement sequence for shRNA designated CD3-gamma_2
cuggcgaacuccauccugucc

SEQ ID NO: 87 - RNA, 21 nt, Artificial sequence
  RNA effector sequence for shRNA designated CD3-gamma_3
guucgccagucgagagcuuca

SEQ ID NO: 88 - RNA, 21 nt, Artificial sequence
  RNA effector complement sequence for shRNA designated CD3-gamma_3
ugaagcucucgacuggcgaac

SEQ ID NO: 89 - RNA, 21 nt, Artificial sequence
  RNA effector sequence for shRNA designated CD3-gamma_4
cagacaagcagacucuguugc

SEQ ID NO: 90 - RNA, 21 nt, Artificial sequence
  RNA effector complement sequence for shRNA designated CD3-gamma_4
gcaacagagucugcuugucug

SEQ ID NO: 91 - RNA, 21 nt, Artificial sequence
  RNA effector sequence for shRNA designated CD3-gamma_5
accagccccucaaggaucgag

SEQ ID NO: 92 - RNA, 21 nt, Artificial sequence
  RNA effector complement sequence for shRNA designated CD3-gamma_5
cucgauccuugaggggcuggu

SEQ ID NO: 93 - RNA, 21 nt, Artificial sequence
  RNA effector sequence for shRNA designated CD3-gamma_6
gagcuucagacaagcagacuc

SEQ ID NO: 94 - RNA, 21 nt, Artificial sequence
  RNA effector complement sequence for shRNA designated CD3-gamma_6
gagucugcuugucugaagcuc

SEQ ID NO: 95 - RNA, 21 nt, Artificial sequence
  RNA effector sequence for shRNA designated CD3-gamma_7
uccaaguguauuacagaaugu

SEQ ID NO: 96 - RNA, 21 nt, Artificial sequence
  RNA effector complement sequence for shRNA designated CD3-gamma_7
acauucuguaauacacuugga

SEQ ID NO: 97 - RNA, 18 nt, Artificial sequence
  RNA stem loop sequence for shmiRs
acugugaagcagaugggu

SEQ ID NO: 98 - RNA, 20 nt, Artificial sequence
  5′ flanking sequence of the pri-miRNA backbone
gguauauugcuguugacagu

SEQ ID NO: 99 - RNA, 22 nt, Artificial sequence
  3′ flanking sequence of the pri-miRNA backbone
cgccuacugccucggacuucaa

SEQ ID NO: 100 - RNA, 21 nt, Artificial sequence
  RNA effector sequence for shmiR designated shmiR-TCR-alpha_1
uguuucaaagcuuuucucgac

SEQ ID NO: 101 - RNA, 20 nt, Artificial sequence
  RNA effector complement sequence for shmiR designated shmiR-TCR-alpha_1
ucgagaaaagcuuugaaaca

SEQ ID NO: 102 - RNA, 21 nt, Artificial sequence
  RNA effector sequence for shmiR designated shmiR-TCR-alpha_2
uuucaaagcuuuucucgacca

SEQ ID NO: 103 - RNA, 20 nt, Artificial sequence
  RNA effector complement sequence for shmiR designated shmiR-TCR-alpha_2
ggucgagaaaagcuuugaaa

SEQ ID NO: 104 - RNA, 21 nt, Artificial sequence
  RNA effector sequence for shmiR designated shmiR-TCR-alpha_3
aaguuuagguucguaucuguu

SEQ ID NO: 105 - RNA, 20 nt, Artificial sequence
  RNA effector complement sequence for shmiR designated shmiR-TCR-alpha_3
acagauacgaaccuaaacuu

SEQ ID NO: 106 - RNA, 21 nt, Artificial sequence
  RNA effector sequence for shmiR designated shmiR-TCR-alpha_4
uuugaaaguuuagguucguau

SEQ ID NO: 107 - RNA, 20 nt, Artificial sequence
  RNA effector complement sequence for shmiR designated shmiR-TCR-alpha_4
uacgaaccuaaacuuucaaa

SEQ ID NO: 108 - RNA, 21 nt, Artificial sequence
  RNA effector sequence for shmiR designated shmiR-TCR-beta_1
ccauucacccaccagcucagc

SEQ ID NO: 109 - RNA, 20 nt, Artificial sequence
  RNA effector complement sequence for shmiR designated shmiR-TCR-beta_1
cugagcuggugggugaaugg

SEQ ID NO: 110 - RNA, 21 nt, Artificial sequence
  RNA effector sequence for shmiR designated shmiR-TCR-beta_2
guggccgagacccucaggcgg

SEQ ID NO: 111 - RNA, 20 nt, Artificial sequence
  RNA effector complement sequence for shmiR designated shmiR-TCR-beta_2
cgccugagggucucggccac

SEQ ID NO: 112 - RNA, 21 nt, Artificial sequence
  RNA effector sequence for shmiR designated shmiR-TCR-beta_3
acuggacuugacagcggaagu

SEQ ID NO: 113 - RNA, 20 nt, Artificial sequence
  RNA effector complement sequence for shmiR designated shmiR-TCR-beta_3
cuuccgcugucaaguccagu

SEQ ID NO: 114 - RNA, 21 nt, Artificial sequence
  RNA effector sequence for shmiR designated shmiR-TCR-beta_4
cuugacagcggaagugguugc

SEQ ID NO: 115 - RNA, 20 nt, Artificial sequence
  RNA effector complement sequence for shmiR designated shmiR-TCR-beta_4
caaccacuuccgcugucaag

SEQ ID NO: 116 - RNA, 21 nt, Artificial sequence
  RNA effector sequence for shmiR designated shmiR-TCR-beta_5
ugacagcggaagugguugcgg

SEQ ID NO: 117 - RNA, 20 nt, Artificial sequence
  RNA effector complement sequence for shmiR designated shmiR-TCR-beta_5
cgcaaccacuuccgcuguca

SEQ ID NO: 118 - RNA, 21 nt, Artificial sequence
  RNA effector sequence for shmiR designated shmiR-CD3-gamma_1
ugaagcucucgacuggcgaac

SEQ ID NO: 119 - RNA, 20 nt, Artificial sequence
  RNA effector complement sequence for shmiR designated shmiR-CD3-gamma_1
uucgccagucgagagcuuca

SEQ ID NO: 120 - RNA, 21 nt, Artificial sequence
  RNA effector sequence for shmiR designated shmiR-CD3-gamma_2
acauucuguaauacacuugga

SEQ ID NO: 121 - RNA, 20 nt, Artificial sequence
  RNA effector complement sequence for shmiR designated shmiR-CD3-gamma_2
ccaaguguauuacagaaugu

SEQ ID NO: 122 - RNA, 21 nt, Artificial sequence
  RNA effector sequence for shmiR designated shmiR-CD3-delta_1
guaucuugaaggggcucacuu

SEQ ID NO: 123 - RNA, 20 nt, Artificial sequence
  RNA effector complement sequence for shmiR designated shmiR-CD3-delta_1
agugagccccuucaagauac

SEQ ID NO: 124 - RNA, 21 nt, Artificial sequence
  RNA effector sequence for shmiR designated shmiR-CD3-delta_2
aaagcagaagacucccaaagc

SEQ ID NO: 125 - RNA, 20 nt, Artificial sequence
  RNA effector complement sequence for shmiR designated shmiR-CD3-delta_2
cuuugggagucuucugcuuu

SEQ ID NO: 126 - RNA, 21 nt, Artificial sequence
  RNA effector sequence for shmiR designated shmiR-CD3-delta_3
uguacugagcaucaucucgau

SEQ ID NO: 127 - RNA, 20 nt, Artificial sequence
  RNA effector complement sequence for shmiR designated shmiR-CD3-delta_3
ucgagaugaugcucaguaca

SEQ ID NO: 128 - RNA, 21 nt, Artificial sequence
  RNA effector sequence for shmiR designated shmiR-CD3-delta_4
aaugacaucagugacaaugau

SEQ ID NO: 129 - RNA, 20 nt, Artificial sequence
  RNA effector complement sequence for shmiR designated shmiR-CD3-delta_4
ucauugucacugaugucauu

SEQ ID NO: 130 - RNA, 21 nt, Artificial sequence
  RNA effector sequence for shmiR designated shmiR-CD3-epsilon_1
auucaggccagaauacagguc

SEQ ID NO: 131 - RNA, 20 nt, Artificial sequence
  RNA complement effector sequence for shmiR designated shmiR-CD3-epsilon_1
accuguauucuggccugaau

SEQ ID NO: 132 - RNA, 21 nt, Artificial sequence
  RNA effector sequence for shmiR designated shmiR-CD3-epsilon_2
gauucaggccagaauacaggu

SEQ ID NO: 133 - RNA, 20 nt, Artificial sequence
  RNA effector complement sequence for shmiR designated shmiR-CD3-epsilon_2
ccuguauucuggccugaauc

SEQ ID NO: 134 - RNA, 21 nt, Artificial sequence
  RNA effector sequence for shmiR designated shmiR-CD3-epsilon_3
uucuucauuaccaucuugccc

SEQ ID NO: 135 - RNA, 20 nt, Artificial sequence
  RNA effector complement sequence for shmiR designated shmiR-CD3-epsilon_3
ggcaagaugguaaugaagaa

SEQ ID NO: 136 - RNA, 107 nt, Artificial sequence
  RNA sequence for shmiR designated shmiR-TCR-alpha_1
gguauauugcuguugacagugagcgaucgagaaaagcuuugaaacaacugugaagcagauggguuguuucaaagcuuuucucgaccgccuacugccucggacuucaa

SEQ ID NO: 137 - RNA, 107 nt, Artificial sequence
  RNA sequence for shmiR designated shmiR-TCR-alpha_2
gguauauugcuguugacagugagcgaggucgagaaaagcuuugaaaacugugaagcagauggguuuucaaagcuuuucucgaccacgccuacugccucggacuucaa

SEQ ID NO: 138 - RNA, 107 nt, Artificial sequence
  RNA sequence for shmiR designated shmiR-TCR-alpha_3
gguauauugcuguugacagugagcguacagauacgaaccuaaacuuacugugaagcagauggguaaguuuagguucguaucuguucgccuacugccucggacuucaa

SEQ ID NO: 139 - RNA, 107 nt, Artificial sequence
  RNA sequence for shmiR designated shmiR-TCR-alpha_4
gguauauugcuguugacagugagcguuacgaaccuaaacuuucaaaacugugaagcagauggguuuugaaaguuuagguucguaucgccuacugccucggacuucaa

SEQ ID NO: 140 - RNA, 107 nt, Artificial sequence
  RNA sequence for shmiR designated shmiR-TCR-beta_1
gguauauugcuguugacagugagcgacugagcuggugggugaauggacugugaagcagauggguccauucacccaccagcucagccgccuacugccucggacuucaa

SEQ ID NO: 141 - RNA, 107 nt, Artificial sequence
  RNA sequence for shmiR designated shmiR-TCR-beta_2
gguauauugcuguugacagugagcgacgccugagggucucggccacacugugaagcagauggguguggccgagacccucaggcggcgccuacugccucggacuucaa

SEQ ID NO: 142 - RNA, 107 nt, Artificial sequence
  RNA sequence for shmiR designated shmiR-TCR-beta_3
gguauauugcuguugacagugagcgucuuccgcugucaaguccaguacugugaagcagauggguacuggacuugacagcggaagucgccuacugccucggacuucaa

SEQ ID NO: 143 - RNA, 107 nt, Artificial sequence
  RNA sequence for shmiR designated shmiR-TCR-beta_4
gguauauugcuguugacagugagcgacaaccacuuccgcugucaagacugugaagcagaugggucuugacagcggaagugguugccgccuacugccucggacuucaa

SEQ ID NO: 144 - RNA, 107 nt, Artificial sequence
  RNA sequence for shmiR designated shmiR-TCR-beta_5
gguauauugcuguugacagugagcgacgcaaccacuuccgcugucaacugugaagcagauggguugacagcggaagugguugcggcgccuacugccucggacuucaa

SEQ ID NO: 145 - RNA, 107 nt, Artificial sequence
  RNA sequence for shmiR designated shmiR-CD3-gamma_1
gguauauugcuguugacagugagcgauucgccagucgagagcuucaacugugaagcagauggguugaagcucucgacuggcgaaccgccuacugccucggacuucaa

SEQ ID NO: 146 - RNA, 107 nt, Artificial sequence
  RNA sequence for shmiR designated shmiR-CD3-gamma_2
gguauauugcuguugacagugagcgaccaaguguauuacagaauguacugugaagcagauggguacauucuguaauacacuuggacgccuacugccucggacuucaa

SEQ ID NO: 147 - RNA, 107 nt, Artificial sequence
  RNA sequence for shmiR designated shmiR-CD3-delta_1
gguauauugcuguugacagugagcguagugagccccuucaagauacacugugaagcagauggguguaucuugaaggggcucacuucgccuacugccucggacuucaa

SEQ ID NO: 148 - RNA, 107 nt, Artificial sequence
  RNA sequence for shmiR designated shmiR-CD3-delta_2
gguauauugcuguugacagugagcgacuuugggagucuucugcuuuacugugaagcagauggguaaagcagaagacucccaaagccgccuacugccucggacuucaa

SEQ ID NO: 149 - RNA, 107 nt, Artificial sequence
  RNA sequence for shmiR designated shmiR-CD3-delta_3
gguauauugcuguugacagugagcguucgagaugaugcucaguacaacugugaagcagauggguuguacugagcaucaucucgaucgccuacugccucggacuucaa

SEQ ID NO: 150 - RNA, 107 nt, Artificial sequence
  RNA sequence for shmiR designated shmiR-CD3-delta_4
gguauauugcuguugacagugagcguucauugucacugaugucauuacugugaagcagauggguaaugacaucagugacaaugaucgccuacugccucggacuucaa

SEQ ID NO: 151 - RNA, 107 nt, Artificial sequence
  RNA sequence for shmiR designated shmiR-CD3-epsilon_1
gguauauugcuguugacagugagcgaaccuguauucuggccugaauacugugaagcagauggguauucaggccagaauacagguccgccuacugccucggacuucaa

SEQ ID NO: 152 - RNA, 107 nt, Artificial sequence
  RNA sequence for shmiR designated shmiR-CD3-epsilon_2
gguauauugcuguugacagugagcguccuguauucuggccugaaucacugugaagcagaugggugauucaggccagaauacaggucgccuacugccucggacuucaa

SEQ ID NO: 153 - RNA, 107 nt, Artificial sequence
  RNA sequence for shmiR designated shmiR-CD3-epsilon_3
gguauauugcuguugacagugagcgaggcaagaugguaaugaagaaacugugaagcagauggguuucuucauuaccaucuugccccgccuacugccucggacuucaa

SEQ ID NO: 154 - DNA, 107 bp, Artificial sequence
  DNA sequence coding for shmiR designated shmiR-TCR-alpha_1
ggtatattgctgttgacagtgagcgatcgagaaaagctttgaaacaactgtgaagcagatgggttgtttcaaagcttttctcgaccgcctactgcctcggacttcaa

SEQ ID NO: 155 - DNA, 107 bp, Artificial sequence
  DNA sequence coding for shmiR designated shmiR-TCR-alpha_2
ggtatattgctgttgacagtgagcgaggtcgagaaaagctttgaaaactgtgaagcagatgggttttcaaagcttttctcgaccacgcctactgcctcggacttcaa

SEQ ID NO: 156 - DNA, 107 bp, Artificial sequence
  DNA sequence coding for shmiR designated shmiR-TCR-alpha_3
ggtatattgctgttgacagtgagcgtacagatacgaacctaaacttactgtgaagcagatgggtaagtttaggttcgtatctgttcgcctactgcctcggacttcaa

SEQ ID NO: 157 - DNA, 70 bp, Artificial sequence
  DNA sequence coding for shmiR designated shmiR-TCR-alpha_4
ggtatattgctgttgacagtgagcgttacgaacctaaactttcaaaactgtgaagcagatgggttttgaa

SEQ ID NO: 158 - DNA, 107 bp, Artificial sequence
  DNA sequence coding for shmiR designated shmiR-TCR-beta_1
ggtatattgctgttgacagtgagcgactgagctggtgggtgaatggactgtgaagcagatgggtccattcacccaccagctcagccgcctactgcctcggacttcaa

SEQ ID NO: 159 - DNA, 107 bp, Artificial sequence
  DNA sequence coding for shmiR designated shmiR-TCR-beta_2
ggtatattgctgttgacagtgagcgacgcctgagggtctcggccacactgtgaagcagatgggtgtggccgagaccctcaggcggcgcctactgcctcggacttcaa

SEQ ID NO: 160 - DNA, 107 bp, Artificial sequence
  DNA sequence coding for shmiR designated shmiR-TCR-beta_3
ggtatattgctgttgacagtgagcgtcttccgctgtcaagtccagtactgtgaagcagatgggtactggacttgacagcggaagtcgcctactgcctcggacttcaa

SEQ ID NO: 161 - DNA, 107 bp, Artificial sequence
  DNA sequence coding for shmiR designated shmiR-TCR-beta_4
ggtatattgctgttgacagtgagcgacaaccacttccgctgtcaagactgtgaagcagatgggtcttgacagcggaagtggttgccgcctactgcctcggacttcaa

SEQ ID NO: 162 - DNA, 107 bp, Artificial sequence
  DNA sequence coding for shmiR designated shmiR-TCR-beta_5
ggtatattgctgttgacagtgagcgacgcaaccacttccgctgtcaactgtgaagcagatgggttgacagcggaagtggttgcggcgcctactgcctcggacttcaa

SEQ ID NO: 163 - DNA, 107 bp, Artificial sequence
  DNA sequence coding for shmiR designated shmiR-CD3-gamma_1
ggtatattgctgttgacagtgagcgattcgccagtcgagagcttcaactgtgaagcagatgggttgaagctctcgactggcgaaccgcctactgcctcggacttcaa

SEQ ID NO: 164 - DNA, 107 bp, Artificial sequence
  DNA sequence coding for shmiR designated shmiR-CD3-gamma_2
ggtatattgctgttgacagtgagcgaccaagtgtattacagaatgtactgtgaagcagatgggtacattctgtaatacacttggacgcctactgcctcggacttcaa

SEQ ID NO: 165 - DNA, 107 bp, Artificial sequence
  DNA sequence coding for shmiR designated shmiR-CD3-delta_1
ggtatattgctgttgacagtgagcgtagtgagccccttcaagatacactgtgaagcagatgggtgtatcttgaaggggctcacttcgcctactgcctcggacttcaa

SEQ ID NO: 166 - DNA, 107 bp, Artificial sequence
  DNA sequence coding for shmiR designated shmiR-CD3-delta_2
ggtatattgctgttgacagtgagcgactttgggagtcttctgctttactgtgaagcagatgggtaaagcagaagactcccaaagccgcctactgcctcggacttcaa

SEQ ID NO: 167 - DNA, 107 bp, Artificial sequence
  DNA sequence coding for shmiR designated shmiR-CD3-delta_3
ggtatattgctgttgacagtgagcgttcgagatgatgctcagtacaactgtgaagcagatgggttgtactgagcatcatctcgatcgcctactgcctcggacttcaa

SEQ ID NO: 168 - DNA, 107 bp, Artificial sequence
  DNA sequence coding for shmiR designated shmiR-CD3-delta_4
ggtatattgctgttgacagtgagcgttcattgtcactgatgtcattactgtgaagcagatgggtaatgacatcagtgacaatgatcgcctactgcctcggacttcaa

SEQ ID NO: 169 - DNA, 107 bp, Artificial sequence
  DNA sequence coding for shmiR designated shmiR-CD3-epsilon_1
ggtatattgctgttgacagtgagcgaacctgtattctggcctgaatactgtgaagcagatgggtattcaggccagaatacaggtccgcctactgcctcggacttcaa

SEQ ID NO: 170 - DNA, 107 bp, Artificial sequence
  DNA sequence coding for shmiR designated shmiR-CD3-epsilon_2
ggtatattgctgttgacagtgagcgtcctgtattctggcctgaatcactgtgaagcagatgggtgattcaggccagaatacaggtcgcctactgcctcggacttcaa

SEQ ID NO: 171 - DNA, 107 bp, Artificial sequence
  DNA sequence coding for shmiR designated shmiR-CD3-epsilon_3
ggtatattgctgttgacagtgagcgaggcaagatggtaatgaagaaactgtgaagcagatgggtttcttcattaccatcttgccccgcctactgcctcggacttcaa

SEQ ID NO: 172 - DNA, 2055 bp, Artificial sequence
  DNA sequence for triple construct pBL513 coding for shmiRs designated shmiR-TCR-alpha, shmiR-TCR-beta and shmiR-CD3-epsilon
gcggccgcgtagtacgatgactagcatgcagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgatcgagaaaagctttgaaacaactgtgaagcagatgggttgtttcaaagcttttctcgaccgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgacgcaaccacttccgctgtcaactgtgaagcagatgggttgacagcggaagtggttgcggcgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagtggtaccggtcctacgcggggccctttacccagggtgccccgggcgctcatttgcatgtcccacccaacaggtaaacctgacaggtcatcgcggccaggtacgacctggcggtcagagcaccaaacatacgagccttgtgatgagttccgttgcatgaaattctcccaaaggctccaagatggacaggaaagggcgcggttcggtcaccgtaagtagaataggtgaaagactcccgtgccttataaggcctgtgggtgacttcttgctagcggtatattgctgttgacagtgagcgaggcaagatggtaatgaagaaactgtgaagcagatgggtttcttcattaccatcttgccccgcctactgcctcggacttcaagaattctgtaccgtatatagcatgactgcggccgc

SEQ ID NO: 173 - DNA, 2055 bp, Artificial sequence
  DNA sequence for triple construct pBL514 coding for shmiRs designated shmiR-TCR-alpha, shmiR-TCR-gamma and shmiR-CD3-epsilon
gcggccgcgtagtacgatgactagcatgcagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgatcgagaaaagctttgaaacaactgtgaagcagatgggttgtttcaaagcttttctcgaccgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgaccaagtgtattacagaatgtactgtgaagcagatgggtacattctgtaatacacttggacgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagtggtaccggtcctacgcggggccctttacccagggtgccccgggcgctcatttgcatgtcccacccaacaggtaaacctgacaggtcatcgcggccaggtacgacctggcggtcagagcaccaaacatacgagccttgtgatgagttccgttgcatgaaattctcccaaaggctccaagatggacaggaaagggcgcggttcggtcaccgtaagtagaataggtgaaagactcccgtgccttataaggcctgtgggtgacttctgctagcggtatattgctgttgacagtgagcgaggcaagatggtaatgaagaaactgtgaagcagatgggtttcttcattaccatcttgccccgcctactgcctcggacttcaagaattcctgtaccgtatatagcatgactgcggccgc

SEQ ID NO: 174 - DNA, 2055 bp, Artificial sequence
  DNA sequence for triple construct pBL515 coding for shmiRs designated shmiR-TCR-alpha, shmiR-TCR-delta and shmiR-CD3-epsilon
gcggccgcgtagtacgatgactagcatgcagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgatcgagaaaagctttgaaacaactgtgaagcagatgggttgtttcaaagcttttctcgaccgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgttcgagatgatgctcagtacaactgtgaagcagatgggttgtactgagcatcatctcgatcgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagtggtaccggtcctacgcggggccctttacccagggtgccccgggcgctcatttgcatgtcccacccaacaggtaaacctgacaggtcatcgcggccaggtacgacctggcggtcagagcaccaaacatacgagccttgtgatgagttccgttgcatgaaattctcccaaaggctccaagatggacaggaaagggcgcggttcggtcaccgtaagtagaataggtgaaagactcccgtgccttataaggcctgtgggtgacttcttgctagcggtatattgctgttgacagtgagcgaggcaagatggtaatgaagaaactgtgaagcagatgggtttcttcattaccatcttgccccgcctactgcctcggacttcaagaattctgtaccgtatatagcatgactgcggccgc

SEQ ID NO: 175 - DNA, 1975 bp, Artificial sequence
  DNA sequence for triple construct pBL516 coding for shmiRs designated shmiR-TCR-beta, shmiR-CD3-gamma and shmiR-CD3-epsilon
ccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgacgcaaccacttccgctgtcaactgtgaagcagatgggttgacagcggaagtggttgcggcgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgaccaagtgtattacagaatgtactgtgaagcagatgggtacattctgtaatacacttggacgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagtggtaccggtcctacgcggggccctttacccagggtgccccgggcgctcatttgcatgtcccacccaacaggtaaacctgacaggtcatcgcggccaggtacgacctggcggtcagagcaccaaacatacgagccttgtgatgagttccgttgcatgaaattctcccaaaggctccaagatggacaggaaagggcgcggttcggtcaccgtaagtagaataggtgaaagactcccgtgccttataaggcctgtgggtgacttcttgctagcggtatattgctgttgacagtgagcgaggcaagatggtaatgaagaaactgtgaagcagatgggtttcttcattaccatcttgccccgcctactgcctcggacttcaagaattctgtaccgtatatagcatgactgcggccgc

SEQ ID NO: 176 - DNA, 2022 bp, Artificial sequence
  DNA sequence for triple construct pBL528 coding for shmiRs designated shmiR-TCR-alpha, shmiR-TCR-beta and shmiR-CD3-epsilon.
gcggccgcgtagtacgatgactagcatgcagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgatcgagaaaagctttgaaacaactgtgaagcagatgggttgtttcaaagcttttctcgaccgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgacgcaaccacttccgctgtcaactgtgaagcagatgggttgacagcggaagtggttgcggcgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagaccggtgatctgctccgtcgccgccgcgccgccatgaaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactcggcttgctagcggtatattgctgttgacagtgagcgaggcaagatggtaatgaagaaactgtgaagcagatgggtttcttcattaccatcttgccccgcctactgcctcggacttcaagaa

SEQ ID NO: 177 - DNA, 2022 bp, Artificial sequence
  DNA sequence for triple construct pBL529 coding for shmiRs designated shmiR-TCR-alpha, shmiR-CD3-gamma and shmiR-CD3-epsilon
gcggccgcgtagtacgatgactagcatgcagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgatcgagaaaagctttgaaacaactgtgaagcagatgggttgtttcaaagcttttctcgaccgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgaccaagtgtattacagaatgtactgtgaagcagatgggtacattctgtaatacacttggacgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagaccggtgatctgctccgtcgccgccgcgccgccatgaaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactcggctgctagcggtatattgctgttgacagtgagcgaggcaagatggtaatgaagaaactgtgaagcagatgggtttcttcattaccatcttgccccgcctactgcctcggacttcaagaat

SEQ ID NO: 178 - DNA, 2017 bp, Artificial sequence
  DNA sequence for triple construct pBL530 coding for shmiRs designated shmiR-TCR-beta, shmiR-CD3-gamma and shmiR-CD3-epsilon
gcggccgcgtagtacgatgactagcatgcagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgacgcaaccacttccgctgtcaactgtgaagcagatgggttgacagcggaagtggttgcggcgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgaccaagtgtattacagaatgtactgtgaagcagatgggtacattctgtaatacacttggacgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagtaccggtgatctgctccgtcgccgccgcgccgccatgaaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactcggctagcggtatattgctgttgacagtgagcgaggcaagatggtaatgaagaaactgtgaagcagatgggtttcttcattaccatcttgccccgcctactgcctcggacttcaag

SEQ ID NO: 179 - DNA, 12415 bp, Artificial sequence
  DNA sequence for triple construct pBL531 coding for shmiRs designated shmiR-TCR-beta, shmiR-CD3-gamma and shmiR-CD3-epsilon, and an anti-CD19 chimeric antigen receptor (CAR)
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgaagcggccgcgtagtacgatgactagcatgcagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaactagtggatccggtatattgctgttgacagtgagcgacgcaaccacttccgctgtcaactgtgaagcagatgggttgacagcggaagtggttgcggcgcctactgcctcggacttcaatttttaagcttagatctgttcggctttacgtcacgcgagggcggcagggaggacggaatggcggggtttggggtgggtccctcctcgggggagccctgggaaaagaggactgcgtgtgggaagagaaggtggaaatggcgttttggttgacatgtgccgcctgcgagcgtgctgcggggaggggccgagggcagattcgggaatgatggcgcggggtgggggcgtgggggctttctcgggagaggcccttccctggaagtttggggtgcgatggtgaggttctcggggcacctctggaggggcctcggcacggaaagcgaccacctgggagggcgtgtggggaccaggttttgcctttagttttgcacacactgtagttcatctttatggagatgctcatggcctcattgaagccccacggatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggatccggtatattgctgttgacagtgagcgaccaagtgtattacagaatgtactgtgaagcagatgggtacattctgtaatacacttggacgcctactgcctcggacttcaatttttaagctttacagctctggtagcggtaaccatgcgtatttgacacacgaaggaactagggaaaaggcattaggtcatttcaagccgaaattcacatgtgctagaatccagattccatgctgaccgatgccccaggatatagaaaatgagaatctggtccttaccttcaagaacattcttaaccgtaatcagcctctggtatcttagctccaccctcactggttttttcttgtttgttgaaccggccaagctgctggcctccctcctcaaccgttctgatcatgcttgctaaaatagtcaaaaccccggccagttaaatatgctttagcctgctttattatgattatttttgttgttttggcaatgacctggctacctgttgtttctcccactaaaactttttaagggcagggaattgatctagaaaaaaaaaagctagtggtaccggtgatctgctccgtcgccgccgcgccgccatgaaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactcggctagcggtatattgctgttgacagtgagcgaggcaagatggtaatgaagaaactgtgaagcagatgggtttcttcattaccatcttgccccgcctactgcctcggacttcaatttttcggaattctgtaccgtatatagcatgactgcggccgcttcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgattcgaattcgccgccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttctgggtgctggtcgttgtgggcggcgtgctggcctgctacagcctgctggtgacagtggccttcatcatcttttgggtgaggagcaagcggagcagactgctgcacagcgactacatgaacatgaccccccggaggcctggccccacccggaagcactaccagccctacgcccctcccagggatttcgccgcctaccggagcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggcagcctgctgacatgtggcgacgtggaagagaaccctggccccatgtggctgcagagcctgctgctcttgggcactgtggcctgcagcatctctcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtaataatctagaccgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagtttttaaagaaattgtatttgttaaatatgtactacaaacttagtagt

SEQ ID NO: 180 - RNA, 1508 nt, Homo sapiens
uuuugaaacccuucaaaggcagagacuuguccagccuaaccugccugcugcuccuagcuccugaggcucagggcccuuggcuucuguccgcucugcucagggcccuccagcguggccacugcucagccaugcuccugcugcucgucccagugcucgaggugauuuuuacccugggaggaaccagagcccagucggugacccagcuuggcagccacgucucugucucugaaggagcccugguucugcugaggugcaacuacucaucgucuguuccaccauaucucuucugguaugugcaauaccccaaccaaggacuccagcuucuccugaaguacacaucagcggccacccugguuaaaggcaucaacgguuuugaggcugaauuuaagaagagugaaaccuccuuccaccugacgaaacccucagcccauaugagcgacgcggcugaguacuucugugcugugagugaucucgaaccgaacagcagugcuuccaagauaaucuuuggaucagggaccagacucagcauccggccaaauauccagaacccugacccugccguguaccagcugagagacucuaaauccagugacaagucugucugccuauucaccgauuuugauucucaaacaaaugugucacaaaguaaggauucugauguguauaucacagacaaaacugugcuagacaugaggucuauggacuucaagagcaacagugcuguggccuggagcaacaaaucugacuuugcaugugcaaacgccuucaacaacagcauuauuccagaagacaccuucuuccccagcccagaaaguuccugugaugucaagcuggucgagaaaagcuuugaaacagauacgaaccuaaacuuucaaaaccugucagugauuggguuccgaauccuccuccugaaaguggccggguuuaaucugcucaugacgcugcggcugugguccagcugagaucugcaagauuguaagacagccugugcucccucgcuccuuccucugcauugccccucuucucccucuccaaacagagggaacucuccuacccccaaggaggugaaagcugcuaccaccucugugcccccccgguaaugccaccaacuggauccuacccgaauuuaugauuaagauugcugaagagcugccaaacacugcugccacccccucuguucccuuauugcugcuugucacugccugacauucacggcagaggcaaggcugcugcagccuccccuggcugugcacauucccuccugcuccccagagacugccuccgccaucccacagaugauggaucuucaguggguucucuugggcucuagguccuggagaauguugugagggguuuauuuuuuuuuaauaguguucauaaagaaauacauaguauucuucuucucaagacguggggggaaauuaucucauuaucgaggcccugcuaugcugugugucugggcguguuguauguccugcugccgaugccuucauuaaaaugauuuggaa

SEQ ID NO: 181 - RNA, 1420 nt, Mus musculus
ggacaacagagcagucauugccaagggaggagcaucucucaguuaauagucuccaggucuuguccacagggagauuuuccugaauacaucucucagccuucucacugccuagccauguuccuagugaccauucugcugcucagcgcguucuucucacugagaggaaacagugcccaguccguggaccagccugaugcucacgucacgcucuaugaaggagccucccuggagcucagaugcaguuauucauacagugcagcaccuuaccucuucugguacgugcaguauccuggccagagccuccaguuucuccucaaauacaucacaggagacgccguuguuaaaggcaccaagggcuuugaggccgaguuuaggaagaguaacuccucuuucaaccugaagaaauccccagcccauuggagcgacucagccaaguacuucugugcacuggagguuuccaauaccgacaaagucgucuuuggaacagggaccagauuacaagucucaccaaacauccagaacccagaaccugcuguguaccaguuaaaagauccucggucucaggacagcacccucugccuguucaccgacuuugacucccaaaucaaugugccgaaaaccauggaaucuggaacguucaucacugacaaaacugugcuggacaugaaagcuauggauuccaagagcaauggggccauugccuggagcaaccagacaagcuucaccugccaagauaucuucaaagagaccaacgccaccuaccccaguucagacguucccugugaugccacguugaccgagaaaagcuuugaaacagauacgaaccuaaacuuucaaaaccugucaguuaugggacuccgaauccuccugcugaaaguagcgggauuuaaccugcucaugacgcugaggcugugguccaguugaggucugcaagacugacagagccugacucccaaguuccguccuccucaccccuccgcucccucuucaagccaaaaggagccggcugucuggggucugguuggcccugauucacaaucccaccuggaucucccagauuugugaggaagguugcuggagagcuaagcgcugcugccgcacccacucagcucccucacugcugcugaccauucacaaaaaaaaaaaaaacggcaggggcggggcuucuccuggaucugaagaccccucccccauggcagacuccccuauaaaaucucuuggagaauguuguaaaaaauauggguuuuuuuuuuuuuggcggguuuacuuuuuuaagcauccauaaagaaaugcauauuacucuuucaucaagguguagaaauuaucucauugucuagacccuccugcuacuguguguauugagccacauuguauauuauucugcugcccaugacaucauuaaaggugauucagaaaaacc

SEQ ID NO: 182 - RNA, 1329 nt, Macaca fascicularis
augcuccugguguucaucccacugcuggggauacauuuuguccugagaacugccagagcccagucagugacccagccugauauccauaucacugucucugaaggagccucacuggaguugagauguaacuauuccuauggggcaacaccuucucucuucugguauguccagucccccggccaaggccuccagcugcuccugaaguacuuuucaggagacacugugguucaaggcauuaaaggcuuugaggcugaauuuaagaggagucaauauuccuucaaccugaggaaacccucugugcauuggagugaugcugcugaguacuucugugcugcaggugccacagugccugggucugcagggggagcugaacacaaacugccuaugcuagagaaguuuucagagacucaguguaucuuccuguggcauuuucaaagaucucuugcuuuuaggaugccugucuucucuugcauugauaguuugauguuucuucauacugcuuugaaaaugggugagcagaggagagaguguggggcugcaucuuccuaccgaucuggcauugcccagaagauaacucaaacccaaccagcaauguucgugcaggaaaaggaggcugugacucuggacugcacauaugacaccagugaucaaaauuacggucuauucugguacaagcaacccagcaguggagagaugauuuuucuuauucuucagaugucuuaugacaagcaaaaugcaacagaaggacgcuacucauugaacuuccagaaggcaagaaaauccgucaaccuugucaucucugcuucacaagugggggacucagcaacguauuucugugcaauguacccgucaggaaacagagcucuugucuuuggaaagggcacaagacuuucugugauuccaaauauccagaacccugacccugccguguaccagcugagaggcucuaaauccaaugacaccucugucugccuauuuacugauuuugauucuguaaugaaugugucacaaagcaaggauucugacgugcauaucacagacaaaacugugcuagacaugaggucuauggacuuuaagagcaacggugcuguggccuggagcaacaaauccgauuuugcauguacaagcgccuucaaggacagcguuauuccagcagacaccuucuuccccggcacagaaagugucugugaugccaaccugguugagaaaagcuuugaaacagauaugaaccuaaacuuucaaaaccugucagugauuggguuccgaauccuccuccugaaaguggccggguuuaaucugcucaugacgcugcggcugugguccagcuga

SEQ ID NO: 183 - RNA, 1151 nt, Homo sapiens
cuggucuagaauauuccacaucugcucucacucugccauggacuccuggaccuucugcugugugucccuuugcauccugguagcgaagcauacagaugcuggaguuauccagucaccccgccaugaggugacagagaugggacaagaagugacucugagauguaaaccaauuucaggccacaacucccuuuucugguacagacagaccaugaugcggggacuggaguugcucauuuacuuuaacaacaacguuccgauagaugauucagggaugcccgaggaucgauucucagcuaagaugccuaaugcaucauucuccacucugaagauccagcccucagaacccagggacucagcuguguacuucugugccagcaguuucucgaccuguucggcuaacuauggcuacaccuucgguucggggaccagguuaaccguuguagaggaccugaacaagguguucccacccgaggucgcuguguuugagccaucagaagcagagaucucccacacccaaaaggccacacuggugugccuggccacaggcuucuuccccgaccacguggagcugagcuggugggugaaugggaaggaggugcacaguggggucagcacagacccgcagccccucaaggagcagcccgcccucaaugacuccagauacugccugagcagccgccugagggucucggccaccuucuggcagaacccccgcaaccacuuccgcugucaaguccaguucuacgggcucucggagaaugacgaguggacccaggauagggccaaacccgucacccagaucgucagcgccgaggccugggguagagcagacuguggcuuuaccucgguguccuaccagcaagggguccugucugccaccauccucuaugagauccugcuagggaaggccacccuguaugcugugcuggucagcgcccuuguguugauggccauggucaagagaaaggauuucugaaggcagcccuggaaguggaguuaggagcuucuaacccgucaugguucaauacacauucuucuuuugccagcgcuucugaagagcugcucucaccucucugcaucccaauagauaucccccuaugugcaugcacaccugcacacucacggcugaaaucucccuaacccagggggac

SEQ ID NO: 184 - RNA, 924 nt, Mus musculus
augaacaaguggguuuucugcuggguaacccuuugucuccuuacuguagagaccacacauggugaugguggcaucauuacucagacacccaaauuccugauuggucaggaagggcaaaaacugaccuugaaaugucaacagaauuucaaucaugauacaauguacugguaccgacaggauucagggaaaggauugagacugaucuacuauucaauaacugaaaacgaucuucaaaaaggcgaucuaucugaaggcuaugaugcgucucgagagaagaagucaucuuuuucucucacugugacaucugcccagaagaacgagauggccguuuuucucugugccagcaguauagggacagaauaugaacaguacuucggucccggcaccaggcucacgguuuuagaggaucugagaaaugugacuccacccaaggucuccuuguuugagccaucaaaagcagagauugcaaacaaacaaaaggcuacccucgugugcuuggccaggggcuucuucccugaccacguggagcugagcuggugggugaauggcaaggagguccacaguggggucagcacggacccucaggccuacaaggagagcaauuauagcuacugccugagcagccgccugagggucucugcuaccuucuggcacaauccucgaaaccacuuccgcugccaagugcaguuccaugggcuuucagaggaggacaauuggccagagggcucacccaaaccugucacacagaacaucagugcagaggccuggggccgagcagacuguggaaucacuucagcauccuaucaucaggggguucugucugcaaccauccucuaugagauccuacuggggaaggccacccuauaugcugugcuggucaguggccuggugcugauggccauggucaagaaaaaaaauuccuga

SEQ ID NO: 185 - RNA, 387 nt, Macaca fascicularis
aggaccugaaaaagguguucccacccaagguugcuguguuugagccaucagaagcagagaucucccacacccaaaaggccacgcuggugugccuggccacaggcuucuaccccgaccacguggagcugagcuggugggugaacgggaaagaggugcacaguggggucagcacggacccacagccccucaaggagcagcccgcccucgaggacuccagauacugccugagcagccgccugagggucucggccaccuucuggcacaacccccgcaaccacuuccgcugccaaguccaguucuaugggcucucggaggaugacgaguggacugaggacagggacaagcccaucacccaaaagaucagcgccgaggucugggguagagcag

SEQ ID NO: 186 - RNA, 549 nt, Homo sapiens
auggaacaggggaagggccuggcuguccucauccuggcuaucauucuucuucaagguacuuuggcccagucaaucaaaggaaaccacuugguuaagguguaugacuaucaagaagaugguucgguacuucugacuugugaugcagaagccaaaaauaucacaugguuuaaagaugggaagaugaucggcuuccuaacugaagauaaaaaaaaauggaaucugggaaguaaugccaaggacccucgagggauguaucaguguaaaggaucacagaacaagucaaaaccacuccaaguguauuacagaaugugucagaacugcauugaacuaaaugcagccaccauaucuggcuuucucuuugcugaaaucgucagcauuuucguccuugcuguuggggucuacuucauugcuggacaggauggaguucgccagucgagagcuucagacaagcagacucuguugcccaaugaccagcucuaccagccccucaaggaucgagaagaugaccaguacagccaccuucaaggaaaccaguugaggaggaauuga

SEQ ID NO: 187 - RNA, 549 nt, Mus musculus
auggagcagaggaagggucuggcuggccucuuccuggugaucucucuucuucaaggcacuguagcccagacaaauaaagcaaagaauuugguacaaguggauggcagccgaggagacgguucuguacuucugacuuguggcuugacugacaagacuaucaaguggcuuaaagacgggagcauaauaaguccucuaaaugcaacuaaaaacacauggaaucugggcaacaaugccaaagacccucgaggcacguaucagugucaaggagcaaaggagacgucaaacccccugcaaguguauuacagaaugugugaaaacugcauugagcuaaacauaggcaccauauccggcuuuaucuucgcugaggucaucagcaucuucuuccuugcucuugguguauaucucauugcgggacaggauggaguucgccagucaagagcuucagacaagcagacucuguugcaaaaugaacagcuguaccagccccucaaggaccgggaauaugaccaguacagccaucuccaaggaaaccaacugaggaagaaguga

SEQ ID NO: 188 - RNA, 546 nt, Macaca fascicularis
augguucaggggaagggccugacuggcuucauccuggcuaucauucuucuucaagguaguuuggcccaaucauuugaagaaaaccgcaagcuuaacguguauaaccaagaagaugguucaguacuucugacuugucaugugaaaaacacaaauaucacaugguuuaaagaagggaagaugauagacauccuaacugcacauaaaaauaaauggaaucugggaaguaauaccaaggacccucgagggguguaucaguguaaaggaucaaaggacaagucaaaaacacuccaaguguauuacagaaugugucagaacugcauugaacuaaaugcagccaccauauugggcuuugucuuugcugaaaucaucagcauuuucuuccuugcuguuggggucuacuucauugcuggacaggauggaguucgccagucgagagcuucagacaagcagacucuguugccuaaugaccagcucuaccagccccucaaggaucgagaagaugaccaguacagucaccuucaagggaaccaguugaggaugaauuga

SEQ ID NO: 189 - RNA, 516 nt, Homo sapiens
auggaacauagcacguuucucucuggccugguacuggcuacccuucucucgcaagugagccccuucaagauaccuauagaggaacuugaggacagaguguuugugaauugcaauaccagcaucacauggguagagggaacggugggaacacugcucucagacauuacaagacuggaccugggaaaacgcauccuggacccacgaggaauauauagguguaaugggacagauauauacaaggacaaagaaucuaccgugcaaguucauuaucgaaugugccagagcuguguggagcuggauccagccaccguggcuggcaucauugucacugaugucauugccacucugcuccuugcuuugggagucuucugcuuugcuggacaugagacuggaaggcugucuggggcugccgacacacaagcucuguugaggaaugaccaggucuaucagccccuccgagaucgagaugaugcucaguacagccaccuuggaggaaacugggcucggaacaaguga

SEQ ID NO: 190 - RNA, 522 nt, Mus musculus
auggaacacagcgggauucuggcuagucugauacugauugcuguucucccccaagggagccccuucaagauacaagugaccgaauaugaggacaaaguauuugugaccugcaauaccagcgucaugcaucuagauggaacgguggaaggaugguuugcaaagaauaaaacacucaacuugggcaaaggcguucuggacccacgagggauauaucuguguaaugggacagagcagcuggcaaagguggugucuucugugcaaguccauuaccgaaugugccagaacuguguggagcuagacucgggcaccauggcuggugucaucuucauugaccucaucgcaacucugcuccuggcuuugggcgucuacugcuuugcaggacaugagaccggaaggccuucuggggcugcugagguucaagcacugcugaagaaugagcagcuguaucagccucuucgagaucgugaagauacccaguacagccgucuuggagggaacuggccccggaacaagaaaucuuaa

SEQ ID NO: 191 - RNA, 516 nt, Macaca fascicularis
auggaacauagcacguuucugucuggccugguacuggcuacccuucucucccaagugagccccuucaagauaccuguagaggaacuugaggacagaguguuugugaaaugcaauaccagcgucacauggguagagggaacggugggaacacugcucacaaauaauacaagacuggaccugggaaaacgcauccuggacccacgaggaauauauagguguaaugggacagauauauacaaggacaaagaaucugcugugcaaguucauuaucgaaugugccagaacuguguggagcuggauccagccacccuggcuggcaucauugucacugaugucauugccacucugcuccuugcuuugggagucuucugcuuugcuggacaugagacuggaaggcucucuggggcugccgacacacaagcucuauugaggaaugaccaggucuaucagccccuccgagaucgagaugaugcucaguacagccgccuuggaggaaacugggcucggaacaaguga

SEQ ID NO: 192 - RNA, 624 nt, Homo sapiens
augcagucgggcacucacuggagaguucugggccucugccucuuaucaguuggcguuugggggcaagaugguaaugaagaaaugggugguauuacacagacaccauauaaagucuccaucucuggaaccacaguaauauugacaugcccucaguauccuggaucugaaauacuauggcaacacaaugauaaaaacauaggcggugaugaggaugauaaaaacauaggcagugaugaggaucaccugucacugaaggaauuuucagaauuggagcaaagugguuauuaugucugcuaccccagaggaagcaaaccagaagaugcgaacuuuuaucucuaccugagggcaagagugugugagaacugcauggagauggaugugaugucgguggccacaauugucauaguggacaucugcaucacugggggcuugcugcugcugguuuacuacuggagcaagaauagaaaggccaaggccaagccugugacacgaggagcgggugcuggcggcaggcaaaggggacaaaacaaggagaggccaccaccuguucccaacccagacuaugagcccauccggaaaggccagcgggaccuguauucuggccugaaucagagacgcaucuga

SEQ ID NO: 193 - RNA, 570 nt, Mus musculus
augcgguggaacacuuucuggggcauccugugccucagccuccuagcuguuggcacuugccaggacgaugccgagaacauugaauacaaagucuccaucucaggaaccaguguagaguugacgugcccucuagacagugacgagaacuuaaaaugggaaaaaaauggccaagagcugccucagaagcaugauaagcaccuggugcuccaggauuucucggaagucgaggacaguggcuacuacgucugcuacacaccagccucaaauaaaaacacguacuuguaccugaaagcucgagugugugaguacuguguggagguggaccugacagcaguagccauaaucaucauuguugacaucuguaucacucugggcuugcugauggucauuuauuacuggagcaagaauaggaaggccaaggccaagccugugacccgaggaaccggugcugguagcaggcccagagggcaaaacaaggagcggccaccaccuguucccaacccagacuaugagcccauccgcaaaggccagcgggaccuguauucuggccugaaucagagagcagucuga

SEQ ID NO: 194 - RNA, 597 nt, Macaca fascicularis
augcagucgggcacucgcuggagaguucugggccucugccucuuaucaauuggcguuugggggcaagaugguaaugaagaaauggguaguauuacacagacaccauaucaagucuccaucucuggaaccacaguaauacugacaugcucucagcaucuuggaucugaagcacaauggcaacacaaugguaaaaacaaaggagauucuggggaucaacuguuucugccggaauuuucagaaauggagcaaagugguuauuaugucugcuaccccagaggaagcaauccagaggacgcgagccaucaucucuaccugaaggcaagagugugugagaacugcauggagauggaugugauggcgguggccacaauugucauaguggacaucugcaucacucugggcuugcugcugcugguuuacuacuggagcaagaauagaaaggccaaggccaagccugugacacgaggagcaggugcuggcggcaggcaaaggggacaaaacaaggagaggccaccaccuguucccaacccagacuaugagcccauccggaaaggccagcaggaucuguauucuggccugaaucagagacgcaucuga